(19) United States
(12) Patent Application Publication
(10) Pub. No.: US 2026/0152734 A1
(43) Pub. Date: Jun. 4, 2026

(54) TREATMENT OF LIMB SPASTICITY

(71) Applicant: IPSEN BIOPHARM LIMITED, Wrexham (GB)

(72) Inventors: Nicolae Grigore, Wrexham (GB); Laurent Pons, Wrexham (GB); Katie Ruffle, Wrexham (GB); Philippe Picault, Wrexham (GB)

(21) Appl. No.: 18/851,590

(22) PCT Filed: Apr. 28, 2023
(86) PCT No.: PCT/GB2023/051129

§ 371 Date: Sep. 26, 2024

(30) Foreign Application Priority Data
Apr. 29, 2022 (GB) 2206348.1

(51) Int. Cl. C12N 9/52; A61K 9/00; A61K 38/00; A61P 21/02; C07K 14/33

(52) U.S. Cl. CPC C12N 9/52; A61K 9/0019; A61P 21/02; C07K 14/33; A61K 38/00; C07K 2319/74; C12Y 304/24069

(57) ABSTRACT

The invention is directed to treatment of limb spasticity using a modified botulinum neurotoxin A (BoNT/A) comprising a BoNT/A light-chain and translocation domain, and a BoNT/B receptor binding domain (HC domain). Also provided are unit dosage forms of said modified BoNT/A and corresponding kits.

### FIELD OF THE INVENTION

The present invention relates to treatment of limb spasticity using a modified botulinum neurotoxin A (BoNT/A).

### BACKGROUND

Spasticity is a motor symptom characterised by an increase in velocity dependent stretch reflexes, with exaggerated tendon jerks, resulting from hyperexcitability of the stretch reflex, as one component of the motor neuron syndrome. Spasticity is associated with various neurological disorders e.g. multiple sclerosis, cerebral or spinal cord injuries, traumatic brain injury (TBI), and cerebrovascular disorders (stroke). It is characterised by motor impairment (increased muscle tone, abnormal limb posture, excessive contraction of antagonist muscles and hyperactive reflexes) functional disability, pain, and discomfort.

Adult upper limb (AUL) spasticity is a common complication after stroke; it is often painful (associated with spastic muscle contraction) and can cause significant disability by interfering with upper limb movement and limiting use of the limb for active functional tasks. In severe cases, it can also impede ‘passive function’, such as washing, dressing and caring for the affected limb, thereby increasing the burden on caregivers. Secondary complications may include poor self-esteem and body image, impaired quality of life (reduced social and family interaction) and pressure ulcers.

Upper limb post-stroke spasticity management is challenging due to the diversity of patient presentation and their goals. In these patients, muscle hypertonia typically manifests in several common clinical patterns determined by the muscles affected, which is in turn related to the size, location and age of the Central Nervous System lesions. These patterns were described in an international, cross-sectional survey of clinicians in 31 countries and subsequently were used to develop a classification of 5 typical patterns for AUL spasticity. These are defined with respect to the position of the shoulder, elbow, forearm, and wrist joints. Current clinical consensus and existing guidelines recommend that the exact pattern of spasticity should be considered when selecting the treatment for patients and botulinum neurotoxin A (BoNT/A) therapy should be tailored accordingly with the appropriate muscles selected for injection. However, no two patients are identical and there is a need for custom/personalised treatment regimens.

An expert panel looked further on these patterns in order to provide guidance of treatment parameters for upper limb spasticity (ULS) and the targeted muscles for each of the clinical patterns (see FIG. 1). Significantly the panel defined three aggregate posture combinations to target the treatment with BoNT. These were:
(1) Adducted shoulder, Flexed elbow, Pronated forearm, Flexed wrist, Clenched fist;
(2) Flexed elbow, Pronated forearm, Flexed wrist, Clenched fist; and
(3) Flexed wrist, Clenched fist.

The four upper limb joints shown in FIG. 1 are known to be involved in >90% of subjects (see FIG. 1). The majority of spastic upper limb patterns (four out of five) result from internal rotation and adduction of the shoulder and flexion at the elbow joint, with differences in posture resulting from the posture of the forearm and the wrist.

Overall ULS treatment is aimed at relieving the signs and symptoms of spasticity—reducing muscle spasms and pain, improving posture, facilitating mobility and dexterity (voluntary motor functions reaching for, grasping, moving and releasing), minimising contractures and deformity and improving patient ease of care as well as hygiene/self-care and/or quality of life. Available pharmacological treatment options like oral (benzodiazepines, baclofen, tizanidine and dantrolene) and intrathecal (baclofen) medications mostly cause nonselective muscle weakness with side effects such as generalised weakness and adverse effects on the central nervous system, including ataxia, drowsiness, sedation and even withdrawal symptoms.

The most effective approach for the treatment of spasticity is a combination of physiotherapy and intramuscular (i.m.) injections of BoNT/A that has been recommended by several guidelines in the recent years. BoNT/A has emerged as a treatment of choice as it has minimal systemic side effects, and being a locally injectable treatment, can be adapted to the individual's disease presentation by selectively targeting the affected muscles.

Dysport® is a medicinal product containing drug substance BoNT/A haemagglutinin complex (BTX-A-HAC) isolated and purified from Clostridium botulinum type A strain. Several other medicinal BoNT/A products naturally produced by Clostridium botulinum are also on the market (e.g. BOTOX® and XEOMIN®).

BoNT/A selectively inhibits the release of acetylcholine from the presynaptic nerve terminals and thus blocks cholinergic transmission at the neuromuscular junction inducing a reduction in the muscle contraction and muscle tone, causing the injected muscles to relax. This mechanism of action has been used therapeutically to treat several clinical neurological conditions including focal dystonias, focal muscle spasticity and aesthetic conditions for more than two decades.

However, the duration of action of the currently available BoNT/A products is about 12 to 14 weeks, which is when the new nerve endings sprout allowing the nerve function to return to normal, and the original symptoms reappear. Consequently, for the effect to be maintained, injections need to be repeated periodically.

Thus, the frequency of BoNT/A injections is an important consideration for the treatment of spasticity, considering the chronicity of the condition and long-term nature of the treatment required. Indeed, it has an impact on the direct and indirect health costs involved for the patients and caregivers, the logistics for injections within the hospitals/clinics, and most importantly the quality of life of patients.

Dysport® is approved for the treatment of upper and lower limb spasticity with a maximum total dose per treatment session of 1,500 Units (see FIG. 2—1,000 Units maximum for treating upper limb spasticity). A clinician is required to administer Dysport® to a plurality of muscles on the limbs up to the upper threshold of 1,500 Units total per treatment session. The clinician is forced to make difficult choices during treatment of a patient. In other words, in conventional treatment regimens, a clinician must find a balance between the relatively low total amount of BoNT/A that can be administered (1,500 Units or 1,000 Units for upper limb spasticity—necessitated by the highly toxic nature of BoNT/A) and the effective amount at a plurality of different muscles. Hence, certain muscles are neglected while others receive a suboptimal amount of BoNT/A, resulting in suboptimal therapy. Furthermore, the current treatment regimens exclude treatment of the shoulder, which requires multiple unit doses at a plurality of muscles thereof.

Moreover, the conventional treatment regimens are complicated and result in clinicians under-dosing in an effort to avoid toxicity to the patient. There is thus a need for a convenient, safe, and effective single dose unit and a corresponding guide to the number of units that can be administered to a limb (including the number of injection sites per muscle) in a treatment session without resultant patient toxicity.

In conclusion, there is a need for an improved treatment for limb spasticity that would allow an individualised patient-centric approach to tailor the treatment according to the targeted clinical pattern permitting different combinations of limb muscles to be injected depending on the distribution, extent and severity of spasticity, while avoiding toxicity and providing a longer-lasting treatment (resulting in less frequent administration). There is also a need for an improved treatment regimen that allows treatment of neglected muscles, such as those of the shoulder.

The present invention overcomes one or more of the above-mentioned problems.

### SUMMARY OF THE INVENTION

The present inventors have surprisingly found that a modified BoNT/A finds particular utility in treating limb spasticity. The modified BoNT/A of the invention comprises a BoNT/A light-chain and translocation domain (H_{N} domain) and a BoNT/B receptor binding domain (H_{C} domain), which results in a modified BoNT/A that exhibits increased retention at (reduced diffusion away from) a site of administration and/or increased duration of action (e.g. 6-9 months). Advantageously, the modified BoNT/A has a safety profile that is improved when compared to unmodified BoNT/A (e.g. Dysport®). This improved safety profile may be expressed by the high Safety Ratio described herein for the modified BoNT/A.

Based on the pre-clinical and clinical data herein, it has been shown that a higher total amount of the modified BoNT/A of the invention can be administered to a subject while achieving a similar safety profile to unmodified BoNT/A (e.g. Dysport®) while at such high doses. Thus, modified BoNT/A of the invention can be injected and/or can be injected at a greater number of muscles/sites in the treatment of limb spasticity before reaching the maximum total dose. This is a significant and advantageous finding, and yields an improved treatment of limb spasticity while providing clinicians with a greater range of treatment options. For the first time, it also provides the option of being able to treat additional large muscles such as those of the shoulder, while also treating the elbow, forearm, and/or wrist well within the maximum dose. The treatment may be improved in that it provides for longer-lasting treatment (resulting in less frequent administration) and/or is capable of being tailored for the subject and/or results in an improved quality of life of a subject when compared to treatment with unmodified BoNT/A (e.g. Dysport®). Hence, the treatment of the invention is improved compared to conventional treatment regimens.

Moreover, the present invention provides a convenient, safe, and effective single unit dose as well as a total (maximum) dosage that can safely be administered in a single treatment. The present invention also provides a corresponding guide to the number of times at which said unit dose can be administered to a limb (e.g. including the number of injection sites per muscle) without resultant patient toxicity. Treatment in accordance with the present invention is thus much less complicated for the clinician and helps avoid under-dosing and/or over-dosing. Furthermore, treatment according to the invention is much more satisfactory to the patient, as it is better tailored to the patient's needs, when compared to conventional limb spasticity treatments.

### DETAILED DESCRIPTION

In one aspect, the invention provides a modified BoNT/A for use in treating limb spasticity (adult limb spasticity), wherein the modified BoNT/A is administered by intramuscular injection to a plurality of affected muscles of a subject,
wherein the modified BoNT/A is administered by way of a unit dose of greater than 17,000 pg of modified BoNT/A at the plurality of affected muscles,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 600,000 pg, and
wherein the modified BoNT/A comprises a BoNT/A light-chain and translocation domain, and a BoNT/B receptor binding domain (H_{C} domain).

In another aspect, the invention provides a modified BoNT/A for use in treating limb spasticity (adult limb spasticity) of a subject for a longer duration than that treated by an unmodified BoNT/A (e.g. SEQ ID NO: 2 [such as a di-chain form of SEQ ID NO: 2]), wherein the modified BoNT/A is administered by intramuscular injection to a plurality of affected muscles of a subject,
wherein the modified BoNT/A is administered by way of a unit dose of greater than 17,000 pg of modified BoNT/A at the plurality of affected muscles,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 600,000 pg, and
wherein the modified BoNT/A comprises a BoNT/A light-chain and translocation domain, and a BoNT/B receptor binding domain (H_{C} domain).

In one aspect, the invention provides a method for treating limb spasticity, the method comprising administering a modified BoNT/A by intramuscular injection to a plurality of affected muscles of a subject,
wherein the modified BoNT/A is administered by way of a unit dose of greater than 17,000 pg of modified BoNT/A at the plurality of affected muscles,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 600,000 pg, and
wherein the modified BoNT/A comprises a BoNT/A light-chain and translocation domain, and a BoNT/B receptor binding domain (H_{C} domain).

In a related aspect, the invention provides a method for treating limb spasticity (adult limb spasticity) of a subject for a longer duration than that treated by an unmodified BoNT/A (e.g. SEQ ID NO: 2 [such as a di-chain form of SEQ ID NO: 2]), the method comprising administering a modified BoNT/A by intramuscular injection to a plurality of affected muscles of a subject,
wherein the modified BoNT/A is administered by way of a unit dose of greater than 17,000 pg of modified BoNT/A at the plurality of affected muscles,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 600,000 pg, and
wherein the modified BoNT/A comprises a BoNT/A light-chain and translocation domain, and a BoNT/B receptor binding domain (H_{C} domain).

In another aspect, the invention provides use of a modified BoNT/A in the manufacture of a medicament for treating limb spasticity (adult limb spasticity), wherein the modified BoNT/A is administered by intramuscular injection to a plurality of affected muscles of a subject,
wherein the modified BoNT/A is administered by way of a unit dose of greater than 17,000 pg of modified BoNT/A at the plurality of affected muscles,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 600,000 pg, and
wherein the modified BoNT/A comprises a BoNT/A light-chain and translocation domain, and a BoNT/B receptor binding domain (H_{C} domain).

In another aspect, the invention provides use of a modified BoNT/A in the manufacture of a medicament for treating limb spasticity (adult limb spasticity) of a subject for a longer duration than that treated by an unmodified BoNT/A (e.g. SEQ ID NO: 2 [such as a di-chain form of SEQ ID NO: 2]), wherein the modified BoNT/A is administered by intramuscular injection to a plurality of affected muscles of a subject,
wherein the modified BoNT/A is administered by way of a unit dose of greater than 17,000 pg of modified BoNT/A at the plurality of affected muscles,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 600,000 pg, and
wherein the modified BoNT/A comprises a BoNT/A light-chain and translocation domain, and a BoNT/B receptor binding domain (H_{C} domain).

The unit dose may be greater than 17,000 pg of modified BoNT/A. An upper limit of the unit dose range may be 40,000, 39,000, 38,000, 37,000, 36,000, 35,000, 30,000, 25,000, 24,000, 22,000, 20,000, or 18,000, pg of modified BoNT/A, preferably the upper limit is 38,000 pg. A lower limit of the unit dose range may be 17,500, 18,000, 20,000, 22,000, 24,000, 25,000, 26,000, 27,000, 28,000, 29,000, 30,000, 35,000, 36,000, 37,000, 38,000 or 39,000 pg of modified BoNT/A, preferably the lower limit is 17,500 pg, 22,753 pg, 22,790 pg or 33,181 pg. Preferably, the unit dose of modified BoNT/A is greater than 17,000 pg up to 40,000 pg of modified BoNT/A, e.g. greater than 17,000 pg up to 36,000 pg, or 20,000 pg to 39,000 pg. A unit dose of modified BoNT/A may be 22,000 to 38,000 pg, preferably 23,000 to 38,000 pg or 34,000 to 38,000 pg. A unit dose of modified BoNT/A may be 24,000 to 36,000 pg or 25,000 to 36,000 pg. The unit dose may be 25,000 pg up to 40,000 pg of modified BoNT/A. In preferred embodiments, a unit dose of modified BoNT/A is 24,000, 25,000, 30,000 or 36,000 pg. In more preferred embodiments, a unit dose of modified BoNT/A is 30,000 or 36,000 pg (e.g. 36,000 pg).

It is more preferred that a unit dose is 30,000 pg to 40,000 pg, such as 35,000 pg to 37,000 pg of modified BoNT/A. More preferably, the unit dose is 36,000 pg of modified BoNT/A.

It is even more preferred that a unit dose is 20,000 pg to 30,000 pg, such as 24,000 pg to 26,000 pg of modified BoNT/A. Most preferably, the unit dose is 25,000 pg of modified BoNT/A.

A total dose administered when carrying out the treatment regimen of the present invention may be up to 600,000 pg. In other words, the total amount of modified BoNT/A administered at a given treatment session may be up to 600,000 pg. The total dose may be up to 580,000, 560,000, 540,000, 520,000, 500,000, 480,000, 460,000, 450,000, 440,000, 420,000, 400,000, 380,000, 360,000, 340,000, 320,000, 300,000, 280,000, 260,000, 240,000, 220,000 or 200,000 pg. Preferably, the total dose may be up to 540,000 pg of modified BoNT/A, more preferably 375,000 pg. The total dose may be at least 17,500, 20,000, 22,500, 25,000, 27,500, 30,000, 35,000, 36,000, 37,000, 38,000, 39,000, or 40,000 pg. The total dose may be at least, 250,000, 255,000, 260,000, 270,000, 280,000, 290,000, 300,000, 320,000, 340,000, 360,000, 380,000, 400,000, 450,000, 500,000, 550,000 or 575,000 pg. Preferably, the total dose may be greater than 255,000 pg or greater than 341,849 pg, more preferably at least 350,000 pg of modified BoNT/A, e.g. at least 497,700 pg or 500,000 pg. The total dose may be 250,000-600,000 pg or 255,000-600,000 pg, preferably greater than 255,000 pg up to 600,000 pg, 350,000-600,000 or 500,000-600,000 pg. In more preferred embodiments, the total dose administered is 520,000-600,000 pg, more preferably 350,000-400,000 pg. The total dose may be 360,000, 450,000 or 540,000 pg, preferably 450,000 or 540,000 pg (e.g. 540,000 pg).

It is more preferred that a total dose is 450,000 pg to 600,000 pg, such as 525,000 pg to 555,000 pg of modified BoNT/A. Most preferably, the total dose is up to 540,000 pg of modified BoNT/A (e.g. the total dose may be 540,000 pg).

It is even more preferred that a total dose is 300,000 pg to 450,000 pg, such as 360,000 pg to 390,000 pg of modified BoNT/A. Most preferably, the total dose is up to 375,000 pg of modified BoNT/A (e.g. the total dose may be 375,000 pg).

Accordingly, the unit dose may be greater than 17,000 pg of modified BoNT/A and the total dose administered when carrying out the treatment regimen of the present invention may be up to 600,000 pg. In a preferable embodiment, the unit dose may be 24,000 pg and the total dose may be 360,000 pg or may be up to 360,000 pg. In another preferable embodiment, the unit dose may be 30,000 pg and the total dose may be 450,000 pg or may be up to 450,000 pg. In another preferable embodiment, the unit dose may be 36,000 pg and the total dose may be 540,000 pg or may be up to 540,000 pg. More preferably, the unit dose may be 25,000 pg and the total dose may be 375,000 pg or may be up to 375,000 pg.

In one aspect, the invention provides a modified BoNT/A for use in treating paediatric limb spasticity, wherein the modified BoNT/A is administered by intramuscular injection to a plurality of affected muscles of a subject,
wherein the modified BoNT/A is administered by way of a unit dose of greater than 8,500 pg of modified BoNT/A at the plurality of affected muscles,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 300,000 pg, and
wherein the modified BoNT/A comprises a BoNT/A light-chain and translocation domain, and a BoNT/B receptor binding domain (H_{C} domain).

In another aspect, the invention provides a modified BoNT/A for use in treating paediatric limb spasticity of a subject for a longer duration than that treated by an unmodified BoNT/A (e.g. SEQ ID NO: 2 [such as a di-chain form of SEQ ID NO: 2]), wherein the modified BoNT/A is administered by intramuscular injection to a plurality of affected muscles of a subject,
wherein the modified BoNT/A is administered by way of a unit dose of greater than 8,500 pg of modified BoNT/A at the plurality of affected muscles,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 300,000 pg, and
wherein the modified BoNT/A comprises a BoNT/A light-chain and translocation domain, and a BoNT/B receptor binding domain (H_{C} domain).

In one aspect, the invention provides a method for treating paediatric limb spasticity, the method comprising administering a modified BoNT/A by intramuscular injection to a plurality of affected muscles of a subject,
wherein the modified BoNT/A is administered by way of a unit dose of greater than 8,500 pg of modified BoNT/A at the plurality of affected muscles,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 300,000 pg, and
wherein the modified BoNT/A comprises a BoNT/A light-chain and translocation domain, and a BoNT/B receptor binding domain (H_{C} domain).

In a related aspect, the invention provides a method for treating paediatric limb spasticity of a subject for a longer duration than that treated by an unmodified BoNT/A (e.g. SEQ ID NO: 2 [such as a di-chain form of SEQ ID NO: 2]), the method comprising administering a modified BoNT/A by intramuscular injection to a plurality of affected muscles of a subject,
wherein the modified BoNT/A is administered by way of a unit dose of greater than 8,500 pg of modified BoNT/A at the plurality of affected muscles,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 300,000 pg, and
wherein the modified BoNT/A comprises a BoNT/A light-chain and translocation domain, and a BoNT/B receptor binding domain (H_{C} domain).

In another aspect, the invention provides use of a modified BoNT/A in the manufacture of a medicament for treating paediatric limb spasticity, wherein the modified BoNT/A is administered by intramuscular injection to a plurality of affected muscles of a subject,
wherein the modified BoNT/A is administered by way of a unit dose of greater than 8,500 pg of modified BoNT/A at the plurality of affected muscles,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 300,000 pg, and
wherein the modified BoNT/A comprises a BoNT/A light-chain and translocation domain, and a BoNT/B receptor binding domain (H_{C} domain).

In another aspect, the invention provides use of a modified BoNT/A in the manufacture of a medicament for treating paediatric limb spasticity of a subject for a longer duration than that treated by an unmodified BoNT/A (e.g. SEQ ID NO: 2 [such as a di-chain form of SEQ ID NO: 2]), wherein the modified BoNT/A is administered by intramuscular injection to a plurality of affected muscles of a subject,
wherein the modified BoNT/A is administered by way of a unit dose of greater than 8,500 pg of modified BoNT/A at the plurality of affected muscles,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 300,000 pg, and
wherein the modified BoNT/A comprises a BoNT/A light-chain and translocation domain, and a BoNT/B receptor binding domain (H_{C} domain).

The terms “treating limb spasticity of a subject for a longer duration than that treated by an unmodified BoNT/A” and “treating paediatric limb spasticity of a subject for a longer duration than that treated by an unmodified BoNT/A” may mean that one or more symptoms of limb spasticity of the subject are reduced for a longer time period following administration of a modified BoNT/A of the invention, when compared to administration of an unmodified BoNT/A. Said duration of action may be at least 1.25×, 1.5×, 1.75×, 2.0×, or 2.25× greater. The duration of action of modified BoNT/A may be between 6 and 9 months. For example, a duration of action may be at least: 4.5 months (from onset), 5.0 months, 5.5 months, 6 months, 6.5 months, 7.0 months, 7.5 months, 8.0 months, 8.5 months or 9.0 months. In particular embodiments, a duration of action may be greater than 9.0 months. Said reduction may be determined by comparison to an equivalent control subject exhibiting equivalent symptoms that has been treated with an unmodified BoNT/A. At a time period where the severity of one or more symptoms of the control subject are substantially the same (e.g. the same) as before unmodified BoNT/A treatment, a subject treated with a modified BoNT/A according to the invention may exhibit an improvement in the equivalent one or more symptoms of at least 5%, 10%, 25%, or 50% when compared to the severity of the one or more symptoms before treatment with the modified BoNT/A. The unmodified BoNT/A is preferably SEQ ID NO: 2 present in a di-chain form.

The unit dose for treating paediatric limb spasticity may be greater than 8,500 pg of modified BoNT/A. An upper limit of the unit dose range may be 20,000, 19,500, 19,000, 18,500, 18,000, 17,500, 15,000, 12,500, 12,000, 11,000, 10,000, or 9,000, pg of modified BoNT/A, preferably the upper limit is 19,000 pg. A lower limit of the unit dose range may be 8,750, 9,000, 10,000, 11,000, 12,000, 12,500, 13,000, 13,500, 14,000, 14,500, 15,000, 17,500, 18,000, 18,500, 19,000 or 19,500 pg of modified BoNT/A, preferably the lower limit is 8,750 pg, 11,376 pg, 11,377 pg, 11,395 pg, 11,400 pg or 16,591 pg. Preferably, the unit dose of modified BoNT/A is greater than 8,500 pg up to 20,000 pg of modified BoNT/A, e.g. greater than 8,500 pg up to 18,000 pg, 10,000 pg to 19,500 pg. A unit dose of modified BoNT/A may be 11,000 pg to 19,000 pg, preferably 11,500 to 19,000 pg or 17,000 to 19,000 pg. A unit dose of modified BoNT/A may be 12,000 pg to 18,000 pg or 12,500 pg to 18,000 pg. The unit dose may be 12,500 pg to 20,000 pg of modified BoNT/A. A unit dose of modified BoNT/A may be 12,000, 12,500, 15,000 or 18,000 pg. In more preferred embodiments, a unit dose of modified BoNT/A is 15,000 or 18,000 pg (e.g. 18,000 pg).

It is more preferred that a unit dose for treating paediatric limb spasticity is 15,000 pg to 20,000 pg, such as 17,500 to 18,500 pg of modified BoNT/A. More preferably, the unit dose for treating paediatric limb spasticity is 18,000 pg of modified BoNT/A.

It is even more preferred that a unit dose for treating paediatric limb spasticity is 10,000 pg to 15,000 pg, such as 12,000 pg to 13,000 pg of modified BoNT/A. More preferably, the unit dose for treating paediatric limb spasticity is 12,500 pg of modified BoNT/A.

A total dose administered when carrying out the paediatric spasticity treatment regimen of the present invention may be up to 300,000 pg. In other words, the total amount of modified BoNT/A administered at a given treatment session may be up to 300,000 pg. The total dose may be up to 290,000, 280,000, 270,000, 260,000, 250,000, 240,000, 230,000, 225,000, 220,000, 210,000, 200,000, 190,000, 180,000, 170,000, 160,000, 150,000, 140,000, 130,000, 120,000, 110,000 or 100,000 pg. Preferably, the total dose may be up to 270,000 pg of modified BoNT/A. The total dose may be at least 8,750, 10,000, 11,250, 12,500, 13,750, 15,000, 17,500, 18,000, 18,500, 19,000, 19,500, or 20,000 pg. The total dose may be at least, 125,000, 127,500, 130,000, 135,000, 140,000, 145,000, 150,000, 160,000, 170,000, 180,000, 190,000, 200,000, 225,000, 250,000, 275,000, 290,000 pg, or 287,500 pg. Preferably, the total dose may be greater than 127,500 pg or greater than 170,924 pg or 170,925 pg, more preferably at least 175,000 pg of modified BoNT/A, e.g. at least 248,850 pg, 248,851 pg or 250,000 pg. The total dose may be 125,000-300,000 pg or 127,500-300,000 pg, preferably greater than 127,500 pg up to 300,000 pg, 175,000-300,000 pg or 250,000-300,000 pg. In more preferred embodiments, the total dose administered is 260,000-300,000 pg, more preferably 175,000-200,000 pg. The total dose may be 180,000, 225,000 or 270,000 pg, preferably 225,000 or 270,000 pg (e.g. 270,000 pg).

It is more preferred that a total dose when treating paediatric limb spasticity is 225,000 pg to 300,000 pg, such as 262,500 pg to 277,500 pg of modified BoNT/A. Most preferably, the total dose when treating paediatric limb spasticity is up to 270,000 pg of modified BoNT/A (e.g. the total dose may be 270,000 pg).

It is even more preferred that a total dose when treating paediatric limb spasticity is 150,000 pg to 225,000 pg, such as 180,000 pg to 195,000 pg of modified BoNT/A. Most preferably, the total dose when treating paediatric limb spasticity is up to 187,500 pg of modified BoNT/A (e.g. the total dose may be 187,500 pg).

Accordingly, the unit dose for treating paediatric limb spasticity may be greater than 8,500 pg of modified BoNT/A and the total dose administered when carrying out the treatment regimen of the present invention for treating paediatric limb spasticity may be up to 300,000 pg. In a preferable embodiment, the unit dose may be 12,000 pg and the total dose may be 180,000 pg or may be up to 180,000 pg. In another preferable embodiment, the unit dose may be 15,000 pg and the total dose may be 225,000 pg or may be up to 225,000 pg. In another preferable embodiment, the unit dose may be 18,000 pg and the total dose may be 270,000 pg or may be up to 270,000 pg. More preferably, the unit dose may be 12,500 pg and the total dose may be 187,500 pg or may be up to 187,500 pg.

A unit dose may be expressed in terms of an amount of modified BoNT/A of the invention, in Units of modified BoNT/A, or a combination thereof. Accordingly, a unit dose of modified BoNT/A may also be expressed in both Units and amounts (pg or ng, preferably pg) simultaneously.

In one aspect the invention provides a modified BoNT/A for use in treating limb spasticity (adult limb spasticity), wherein the modified BoNT/A is administered by intramuscular injection to a plurality of affected muscles of a subject,
wherein the modified BoNT/A is administered by way of a unit dose of greater than 707 Units of modified BoNT/A at the plurality of affected muscles, and wherein 1 Unit is an amount of the modified BoNT/A that corresponds to the calculated median lethal dose (LD₅₀) in mice,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 24,958 Units, and
wherein the modified BoNT/A comprises a BoNT/A light-chain and translocation domain, and a BoNT/B receptor binding domain (H_{C} domain).

In a related aspect, the invention provides a modified BoNT/A for use in treating limb spasticity (adult limb spasticity) of a subject for a longer duration than that treated by an unmodified BoNT/A (e.g. SEQ ID NO: 2 [such as a di-chain form of SEQ ID NO: 2]), wherein the modified BoNT/A is administered by intramuscular injection to a plurality of affected muscles of a subject,
wherein the modified BoNT/A is administered by way of a unit dose of greater than 707 Units of modified BoNT/A at the plurality of affected muscles, and wherein 1 Unit is an amount of the modified BoNT/A that corresponds to the calculated median lethal dose (LD₅₀) in mice,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 24,958 Units, and
wherein the modified BoNT/A comprises a BoNT/A light-chain and translocation domain, and a BoNT/B receptor binding domain (H_{C} domain).

In a related aspect the invention provides a method for treating limb spasticity (adult limb spasticity), the method comprising administering a modified BoNT/A by intramuscular injection to a plurality of affected muscles of a subject,
wherein the modified BoNT/A is administered byway of a unit dose of greater than 707 Units of modified BoNT/A at the plurality of affected muscles, and wherein 1 Unit is an amount of the modified BoNT/A that corresponds to the calculated median lethal dose (LD₅₀) in mice,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 24,958 Units, and
wherein the modified BoNT/A comprises a BoNT/A light-chain and translocation domain, and a BoNT/B receptor binding domain (H_{C} domain).

In a related aspect the invention provides a method for treating limb spasticity of a subject for a longer duration than that treated by an unmodified BoNT/A (e.g. SEQ ID NO: 2 [such as a di-chain form of SEQ ID NO: 2]), the method comprising administering a modified BoNT/A by intramuscular injection to a plurality of affected muscles of a subject,
wherein the modified BoNT/A is administered by way of a unit dose of greater than 707 Units of modified BoNT/A at the plurality of affected muscles, and wherein 1 Unit is an amount of the modified BoNT/A that corresponds to the calculated median lethal dose (LD₅₀) in mice,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 24,958 Units, and
wherein the modified BoNT/A comprises a BoNT/A light-chain and translocation domain, and a BoNT/B receptor binding domain (H_{C} domain).

In another related aspect, the invention provides use of a modified BoNT/A in the manufacture of a medicament for treating limb spasticity (adult limb spasticity), wherein the modified BoNT/A is administered by intramuscular injection to a plurality of affected muscles of a subject,
wherein the modified BoNT/A is administered by way of a unit dose of greater than 707 Units of modified BoNT/A at the plurality of affected muscles, and wherein 1 Unit is an amount of the modified BoNT/A that corresponds to the calculated median lethal dose (LD₅₀) in mice,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 24,958 Units, and
wherein the modified BoNT/A comprises a BoNT/A light-chain and translocation domain, and a BoNT/B receptor binding domain (H_{C} domain).

In another related aspect, the invention provides use of a modified BoNT/A in the manufacture of a medicament for treating limb spasticity (adult limb spasticity) of a subject for a longer duration than that treated by an unmodified BoNT/A (e.g. SEQ ID NO: 2 [such as a di-chain form of SEQ ID NO: 2]), wherein the modified BoNT/A is administered by intramuscular injection to a plurality of affected muscles of a subject,
wherein the modified BoNT/A is administered by way of a unit dose of greater than 707 Units of modified BoNT/A at the plurality of affected muscles, and wherein 1 Unit is an amount of the modified BoNT/A that corresponds to the calculated median lethal dose (LD₅₀) in mice,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 24,958 Units, and
wherein the modified BoNT/A comprises a BoNT/A light-chain and translocation domain, and a BoNT/B receptor binding domain (H_{C} domain).

The unit dose may be greater than 707 Units of modified BoNT/A. An upper limit of the unit dose range may be 1664, 1650, 1600, 1550,1500,1450, 1400, 1350, 1300, 1250,1150, 1100, 1050, 1000, 950, 900, 850, 800 or 750 Units of modified BoNT/A, preferably the upper limit is 1500 Units. A lower limit of the unit dose range may be 725, 750, 775, 800, 825, 850, 875, 900, 925, 950, 975,1000,1025,1050,1075,1100,1250,1300,1350,1400,1450,1500, 1550, 1600 or 1650 Units of modified BoNT/A, preferably the lower limit is 725 Units, 949 Units, or 1381 Units. Preferably, the unit dose of modified BoNT/A is greater than 707 Units up to 1664 Units of modified BoNT/A, for example greater than 707 Units up to 1,498 Units or 832 Units to 1622 Units. A unit dose of modified BoNT/A may be 915 to 1581 Units, preferably 949 to 1581 Units or 1414 to 1581 Units. A unit dose of modified BoNT/A may be 998 to 1,498 Units or 1,040 to 1,498 Units. In preferred embodiments, a unit dose of modified BoNT/A is 998, 1,040, 1,248 or 1,498 Units of modified BoNT/A. In more preferred embodiments, a unit dose comprises 1,248 or 1,498 Units (e.g. 1,498 Units) of modified BoNT/A.

It is more preferred that a unit dose is 1,248 Units to 1,664 Units, such as 1,456 Units to 1,539 Units of modified BoNT/A. More preferably, the unit dose is 1,498 Units of modified BoNT/A.

It is even more preferred that a unit dose is 832 Units to 1,248 Units, such as 998 Units to 1,082 Units of modified BoNT/A. Most preferably, the unit dose is 1,040 Units of modified BoNT/A.

A total dose administered when carrying out the treatment regimen of the present invention may be up to 24,958 Units. In other words, the total amount of modified BoNT/A administered at a given treatment session may be up to 24,958 Units. The total dose may be up to 24,500, 24,000, 23,500, 23,000, 22,500, 22,000, 21,500, 21,000, 20,500, 20,000, 19,500, 19,000, 18,500, 18,000, 17,500, 17,000, 16,500, 16,000, 15,500, 15,000, 14,500, 14,000, 13,500, 13,000, 12,500, 12,000, 11,500, 11,000, 10,500, 10,000, 9,500, 9,000 or 8,500 Units. Preferably, the total dose may be up to 22,463 Units of modified BoNT/A, more preferably 15,599 Units. The total dose may be at least 725, 750, 775, 800, 825, 850, 875, 900, 925, 950, 975, 1000, 1,100, 1,200, 1,300, 1,400, 1,500 or 1,600 Units. The total dose may be at least 10,000, 11,000, 12,000, 13,000, 14,000, 15,000, 16,000, 17,000, 18,000, 19,000, 20,000, 21,000, 22,000, 23,000, 24,000 or 24,500 Units. Preferably, the total dose may be greater than 10,607 Units or greater than 14,220 Units, more preferably at least 14,559 Units of modified BoNT/A, e.g. at least 20,703 Units or 20,799 Units. The total dose may be 10,339-24,958 Units, 10,399-24,958 Units or 10,607-24,958 Units, preferably greater than 10,607 Units up to 24,958 Units, 14,559-24,958 Units or 20,799-24,958 Units. More preferably, the total dose administered is 21,631-24,958 Units, more preferably 14,559-16,639 Units. The total dose may be 14,975 Units, 18,719 Units or 22,463 Units, preferably 18,719 or 22,463 Units (e.g. 22,463 Units).

It is more preferred that a total dose is 18,719 Units to 24,958 Units, such as 21,839 Units to 23,087 Units of modified BoNT/A. Most preferably, the total dose is up to 22,463 Units of modified BoNT/A (e.g. the total dose may be 22,463 Units).

It is even more preferred that a total dose is 12,479 Units to 18,719 Units, such as 14,975 Units to 16,223 Units of modified BoNT/A. Most preferably, the total dose is up to 15,599 Units of modified BoNT/A (e.g. the total dose may be 15,599 Units).

Accordingly, the unit dose may be greater than 707 Units of modified BoNT/A and the total dose administered when carrying out the treatment regimen of the present invention may be up to 24,958 Units. In a preferable embodiment, the unit dose may be 998 Units of modified BoNT/A and the total dose may be 14,975 Units or may be up to 14,975 Units. In another preferable embodiment, the unit dose may be 1248 Units of modified BoNT/A and the total dose may be 18,719 Units or may be up to 18,719 Units. In another preferable embodiment, the unit dose may be 1498 Units of modified BoNT/A and the total dose may be 22,463 Units or may be up to 22,463 Units. More preferably, the unit dose may be 1,040 Units of modified BoNT/A and the total dose may be 15,599 Units or may be up to 15,599 Units.

In one aspect the invention provides a modified BoNT/A for use in treating paediatric limb spasticity, wherein the modified BoNT/A is administered by intramuscular injection to a plurality of affected muscles of a subject,
wherein the modified BoNT/A is administered by way of a unit dose of greater than 353.5 Units of modified BoNT/A at the plurality of affected muscles, and wherein 1 Unit is an amount of the modified BoNT/A that corresponds to the calculated median lethal dose (LD₅₀) in mice,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 12,479 Units, and
wherein the modified BoNT/A comprises a BoNT/A light-chain and translocation domain, and a BoNT/B receptor binding domain (H_{C} domain).

In another related aspect, the invention provides a modified BoNT/A for use in treating paediatric limb spasticity of a subject for a longer duration than that treated by an unmodified BoNT/A (e.g. SEQ ID NO: 2 [such as a di-chain form of SEQ ID NO: 2]), wherein the modified BoNT/A is administered by intramuscular injection to a plurality of affected muscles of a subject,
wherein the modified BoNT/A is administered by way of a unit dose of greater than 353.5 Units of modified BoNT/A at the plurality of affected muscles, and wherein 1 Unit is an amount of the modified BoNT/A that corresponds to the calculated median lethal dose (LD₅₀) in mice,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 12,479 Units, and
wherein the modified BoNT/A comprises a BoNT/A light-chain and translocation domain, and a BoNT/B receptor binding domain (H_{C} domain).

In a related aspect the invention provides a method for treating paediatric limb spasticity, the method comprising administering a modified BoNT/A by intramuscular injection to a plurality of affected muscles of a subject,
wherein the modified BoNT/A is administered by way of a unit dose of greater than 353.5 Units of modified BoNT/A at the plurality of affected muscles, and wherein 1 Unit is an amount of the modified BoNT/A that corresponds to the calculated median lethal dose (LD₅₀) in mice,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 12,479 Units, and
wherein the modified BoNT/A comprises a BoNT/A light-chain and translocation domain, and a BoNT/B receptor binding domain (H_{C} domain).

In a related aspect the invention provides a method for treating paediatric limb spasticity of a subject for a longer duration than that treated by an unmodified BoNT/A (e.g. SEQ ID NO: 2 [such as a di-chain form of SEQ ID NO: 2]), the method comprising administering a modified BoNT/A by intramuscular injection to a plurality of affected muscles of a subject,
wherein the modified BoNT/A is administered by way of a unit dose of greater than 353.5 Units of modified BoNT/A at the plurality of affected muscles, and wherein 1 Unit is an amount of the modified BoNT/A that corresponds to the calculated median lethal dose (LD₅₀) in mice,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 12,479 Units, and
wherein the modified BoNT/A comprises a BoNT/A light-chain and translocation domain, and a BoNT/B receptor binding domain (H_{C} domain).

In another related aspect, the invention provides use of a modified BoNT/A in the manufacture of a medicament for treating paediatric limb spasticity, wherein the modified BoNT/A is administered by intramuscular injection to a plurality of affected muscles of a subject,
wherein the modified BoNT/A is administered by way of a unit dose of greater than 353.5 Units of modified BoNT/A at the plurality of affected muscles, and wherein 1 Unit is an amount of the modified BoNT/A that corresponds to the calculated median lethal dose (LD₅₀) in mice,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 12,479 Units, and
wherein the modified BoNT/A comprises a BoNT/A light-chain and translocation domain, and a BoNT/B receptor binding domain (H_{C} domain).

In another related aspect, the invention provides use of a modified BoNT/A in the manufacture of a medicament for treating paediatric limb spasticity of a subject for a longer duration than that treated by an unmodified BoNT/A (e.g. SEQ ID NO: 2 [such as a di-chain form of SEQ ID NO: 2]), wherein the modified BoNT/A is administered by intramuscular injection to a plurality of affected muscles of a subject,
wherein the modified BoNT/A is administered by way of a unit dose of greater than 353.5 Units of modified BoNT/A at the plurality of affected muscles, and wherein 1 Unit is an amount of the modified BoNT/A that corresponds to the calculated median lethal dose (LD₅₀) in mice,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius; and
wherein a single unit dose is administered (preferably at a selected injection site) at an affected first group muscle and/or multiple unit doses are administered (preferably at selected different injection sites) at an affected second group muscle, and wherein the total dose administered during the treatment is up to 12,479 Units, and
wherein the modified BoNT/A comprises a BoNT/A light-chain and translocation domain, and a BoNT/B receptor binding domain (H_{C} domain).

A suitable unit dose for treating paediatric limb spasticity may be greater than 353.5 Units of modified BoNT/A. An upper limit of the unit dose range for treating paediatric limb spasticity may be 832, 825, 800, 775, 750, 725, 700, 675, 650, 625, 600, 575, 550, 525, 500, 475, 450, or 425 Units of modified BoNT/A, preferably the upper limit is 790 Units. A lower limit of the unit dose range for treating paediatric limb spasticity may be 364, 375, 400, 425, 450, 475, 500, 525, 550, 575, 600, 625, 650, 675, 700, 725, 750, 775, 800, 825 or 830 Units of modified BoNT/A, preferably the lower limit is 364 Units, 475 Units, or 690 Units. Preferably, the unit dose of modified BoNT/A for treating paediatric limb spasticity is greater than 353.5 Units up to 832 Units of modified BoNT/A, for example greater than 353.5 Units up to 749 Units or 416 Units to 811 Units. A unit dose of modified BoNT/A for treating paediatric limb spasticity may be 458 Units to 790 Units or 458 Units to 791 Units, preferably 475 Units to 790 Units, 475 Units to 791 Units, 707 Units to 790 Units, or 707 Units to 791 Units. In preferred embodiments, a unit dose of modified BoNT/A is 499, 520, 624 or 749 Units. In more preferred embodiments, a unit dose of modified BoNT/A is 624 or 749 Units (e.g. 749 Units).

It is more preferred that a unit dose for treating paediatric limb spasticity is 624 Units to 832 Units, such as 728 Units to 770 Units of modified BoNT/A. More preferably, the unit dose for treating paediatric limb spasticity is 749 Units of modified BoNT/A.

It is even more preferred that a unit dose for treating paediatric limb spasticity is 416 Units to 624 Units, such as 499 Units to 541 Units of modified BoNT/A. Most preferably, the unit dose for treating paediatric limb spasticity is 520 Units of modified BoNT/A.

A total dose administered when carrying out the treatment regimen of the present invention for treating paediatric limb spasticity may be up to 12,479 Units. In other words, the total amount of modified BoNT/A administered at a given treatment session for treating paediatric limb spasticity may be up to 12,479 Units. The total dose for treating paediatric limb spasticity may be up to 12,250, 12,000, 11,750, 11,500, 11,250, 11,000, 10,750, 10,500, 10,000, 9,750, 9,500, 9,250, 9,000, 8,750, 8,500, 8,250, 8,000, 7,750, 7,500, 7,250, 7,000, 6,750, 6,500, 6,250, 6,000, 5,750, 5,500, 5,250, 5,000, 4,750, 4,500 or 4,250 Units. The total dose for treating paediatric limb spasticity may be up to 11,250 Units of modified BoNT/A. Preferably, the total dose for treating paediatric limb spasticity may be up to 11,232 Units of modified BoNT/A, more preferably 7,800 Units. The total dose for treating paediatric limb spasticity may be at least 350, 375, 400, 425, 450, 475, 500, 525, 550, 575, 600, 625, 650, 675, 700, 725, 750, 775, 800, 825, or 850 Units. The total dose may be at least 5,000, 5,500, 6,000, 6,500, 7,000, 7,500, 8,000, 8,500, 9,000, 9,500, 10,000, 10,500, 11,000 or 11,500 Units. Preferably, the total dose for treating paediatric limb spasticity may be at least 5,304 Units or greater than 7,110 Units, more preferably at least 7,280 Units of modified BoNT/A, e.g. at least 10,351 Units or 10,400 Units. The total dose for treating paediatric limb spasticity may be 5,200-12,479 Units or 5,304-12,479 Units, preferably greater than 5,200 Units up to 12,479 Units, greater than 5,301 Units up to 12,479 Units, 7,280-12,479 Units or 10,400-12,479 Units. In more preferred embodiments, the total dose administered is 10,815-12,479 Units or 10,816-12,479 Units, more preferably 7,280-8,320 Units. The total dose may be 7,488, 9,360, 9,359, 11,231 or 11,232 Units. In more preferred embodiments, the total dose is 9,359 or 11,231 Units (e.g. 11,231 Units).

It is more preferred that a total dose when treating paediatric limb spasticity is 9,360 Units to 12,479 Units, such as 10,920 Units to 11,544 Units of modified BoNT/A. Most preferably, the total dose when treating paediatric limb spasticity is up to 11,232 Units of modified BoNT/A (e.g. the total dose may be 11,232 Units).

It is even more preferred that a total dose when treating paediatric limb spasticity is 6,240 Units to 9,360 Units, such as 7,488 Units to 8,112 Units of modified BoNT/A. Most preferably, the total dose when treating paediatric limb spasticity is up to 7,800 Units of modified BoNT/A (e.g. the total dose may be 7,800 Units).

Accordingly, the unit dose for treating paediatric limb spasticity may be greater than 353.5 Units of modified BoNT/A and the total dose administered when carrying out the treatment regimen of the present invention for treating paediatric limb spasticity may be up to 12,479 Units. In a preferable embodiment, the unit dose may be 499 Units of modified BoNT/A and the total dose may be 7,488 Units or may be up to 7,488 Units. In another preferable embodiment, the unit dose may be 624 Units of modified BoNT/A and the total dose may be 9,359 Units or 9,360 Units or may be up to 9,359 Units or may be up to 9,360 Units. In another preferable embodiment, the unit dose may be 749 Units of modified BoNT/A and the total dose may be 11,231 Units or may be up to 11,231 Units. In another preferable embodiment, the unit dose may be 749 Units of modified BoNT/A and the total dose may be 11,232 Units or may be up to 11,232 Units. Most preferably, the unit dose may be 520 Units and the total dose may be 7,800 Units or may be up to 7,800 Units.

An “affected muscle” is a muscle exhibiting a symptom of spasticity or contributing to the spasticity of an affected limb. For example, said muscle may exhibit increased muscle tone or stiffness.

The plurality of affected muscles are selected from a first and second group as described herein. The plurality of affected muscles selected may be at least one muscle of the first group and/or at least one muscle of the second group. Alternatively, a plurality of affected muscles may be two or more muscles from the same group (e.g. two or more first group muscles or two or more second group muscles). Preferably, the plurality of affected muscles include at least one first group muscle and at least one second group muscle.

The plurality of muscles may be muscles of the same limb or muscles of different limbs. It is preferred, however, that the plurality of muscles are muscles of the same limb. The present invention encompasses treating spasticity in one or more limbs simultaneously. For example, a modified BoNT/A may be administered to one or both upper limbs of a subject per treatment session, one or both lower limbs per treatment session, or a combination of lower and upper limbs per treatment session. Regardless of whether two or more limbs are treated, it is preferred that at least 2 muscles are treated per limb, e.g. at least 3, 4 or 5 muscles per limb.

Potency of a modified BoNT/A for use according to the invention may be determined by a mouse LD₅₀ assay according to standard techniques. In said assay, 1 Unit is defined as an amount of the modified BoNT/A that corresponds to the calculated median lethal dose (LD₅₀) in mice. Preferably, the calculated median lethal intraperitoneal dose in mice.

In particular, an amount of a modified BoNT/A that corresponds to 1 Unit in said assay is preferably 24.04 pg.

Preferably, a dose of modified BoNT/A is administered by intramuscular injection at an affected muscle. More preferably, a single unit dose is administered per injection site. The term “a single unit dose is administered” means substantially all of a single unit dose is administered. For example, a residual amount (e.g. up to 1%, 0.1% or 0.01%) of the unit dose may remain in a vial in which the modified BoNT/A has been reconstituted. However, preferably all of a single unit dose is administered (e.g. at one or more injection sites). Depending on the nature of the muscle, either a single unit dose is administered (i.e. to a muscle selected from a first group described herein) or multiple unit doses are administered (i.e. to a muscle selected from a second group described herein). Said single unit dose or multiple unit doses may be administered at one or more injection sites (e.g. per muscle). For example, in some embodiments, less than a single unit dose may be administered per injection site. In a preferred embodiment, some muscles are injected at one site only (i.e. muscles selected from a first group described herein) and some muscles are injected at two or more sites (i.e. muscles selected from a second group described herein).

In one embodiment, a single unit dose is administered at a plurality of injection sites at an affected first group muscle and/or multiple unit doses are administered at a plurality of injection sites at an affected second group muscle.

The term “up to” when used in reference to a value (e.g. up to 600,000 pg) means up to and including the value recited. Thus, as an example, reference to administering “up to 600,000 pg” of a modified BoNT/A encompasses administration of 600,000 pg of the modified BoNT/A as well as administration of less than 600,000 pg of the modified BoNT/A.

A single unit dose of modified BoNT/A is administered to a muscle selected from the first group described herein when said first group muscle is selected for treatment.

Multiple unit doses of modified BoNT/A are administered to a muscle selected from the second group (e.g. when said second group muscles is selected for treatment). For example, at least 2×, 3× or 4× unit doses may be administered. In some embodiments 2-4× unit doses may be administered. Preferably, 2× unit doses of modified BoNT/A are administered to a muscle selected from the second group.

Some muscles can be present in the first muscle group and second muscle group. Thus, a clinician can decide whether to administer a single unit dose to said muscle or multiple unit doses.

The limb spasticity may be upper limb spasticity or lower limb spasticity.

When treating lower limb spasticity, modified BoNT/A may be administered to a plurality of muscles selected from: a first group comprising (preferably consisting of): the gastrocnemius medial head, the gastrocnemius lateral head, the gastrocnemius, the flexor digitorum longus, the flexor hallucis longus, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and a second group comprising (preferably consisting of): the soleus, the tibialis posterior, the iliopsoas, and the gastrocnemius.

When administering to the flexor hallucis longus, in some instances a single unit dose may be administered, while in other instances 2× the unit dose may be administered.

Suitable dosages by lower limb muscle are shown below:

| Muscles Injected | Dosage |
|---|---|
| Gastrocnemius (Medial head) | 1x UD |
| Gastrocnemius (Lateral head) | 1x UD |
| Soleus | 2x UD or 3x UD |
| Tibialis posterior | 2x UD |
| Flexor digitorum longus (FDL) | 1x UD |
| Flexor hallucis longus | 1x or 2x UD |

The modified BoNT/A may be administered to one or more of the muscles indicated at the dosages indicated in the table above.

Modified BoNT/A may be administered to one or more of the following muscles as follows at the following dosages:

| Muscles Injected | Dosage |
|---|---|
| Adductor magnus | 1x UD |
| Adductor longus | 1x UD |
| Adductor brevis | 1x UD |
| Gracilis | 1x UD |
| Medial hamstrings | 1x UD |
| Lateral hamstrings | 1x UD |
| Tensor fascia lata | 1x UD |
| Rectus femoris | 1x UD |
| Vastus lateralis | 1x UD |
| Vastus medialis | 1x UD |
| Vastus intermedius | 1x UD |
| Gluteus maximus | 1x UD |
| Tibialis anterior | 1x UD |
| FDL | 1x UD |
| Flexor digitorum brevis (FDB) | 1x UD |
| Flexor hallucis longus | 1x UD |
| Extensor hallucis longus | 1x UD |
| Flexor hallucis brevis | 1x UD |
| Iliopsoas | 2x UD |
| Gastrocnemius | 2x UD |
| Tibialis posterior | 2x UD |
| Soleus | 2x UD |

Preferably, when treating lower limb spasticity, modified BoNT/A may be administered to a plurality of muscles selected from: a first group comprising (preferably consisting of): the gastrocnemius medial head (a.k.a. the medial gastrocnemius), the gastrocnemius lateral head (a.k.a. the lateral gastrocnemius), the tibialis anterior, the flexor digitorum longus, the flexor digitorum brevis, the flexor hallucis longus, flexor hallucis brevis, the gracilis, and the gluteus maximus; and a second group comprising (preferably consisting of): the soleus, the tibialis posterior, the rectus femoris, a hamstring muscle (e.g. the biceps femoris, the semimembranosus, and/or the semitendinosus), and the adductor magnus.

A hamstring muscle may be the biceps femoris, the semimembranosus, and/or the semitendinosus. For example, modified BoNT/A may be administered to one or more (e.g. all) of the biceps femoris, the semimembranosus, and/or the semitendinosus.

Preferably, three unit doses are administered to the soleus. Preferably, two unit doses are administered to the tibialis posterior. Preferably, two unit doses are administered to the rectus femoris. Preferably, two unit doses are administered to a hamstring muscle, for example two units may be administered to the biceps femoris, two units may be administered to the semimembranosus, and/or two units may be administered to the semitendinosus). Preferably, two unit doses are administered to the adductor magnus.

Modified BoNT/A may be administered to one or more of the following muscles as follows at the following dosages:

| Muscles Injected | Dosage |
|---|---|
| Medial gastrocnemius | 1x UD |
| Lateral gastrocnemius | 1x UD |
| Tibialis anterior | 1x UD |
| Flexor digitorum longus | 1x UD |
| Flexor digitorum brevis | 1x UD |
| Flexor hallucis longus | 1x UD |
| Flexor hallucis brevis | 1x UD |
| Gracilis | 1x UD |
| Gluteus maximus | 1x UD |
| Soleus | 3x UD |
| Tibialis posterior | 2x UD |
| Rectus Femoris | 2x UD |
| A hamstring muscle | 2x UD |
| Adductor magnus | 2x UD |

Preferably, the limb spasticity is upper limb spasticity.

When treating upper limb spasticity, modified BoNT/A may be administered to a plurality of affected muscles selected from: a first group comprising (preferably consisting of): the flexor digitorum superficialis, the flexor digitorum profundus, the flexor carpi radialis, the flexor carpi ulnaris, the brachioradialis, the pronator teres, the deltoid, the levator scapulae, the pronator quadratus, the flexor policis longus, the adductor policis, the flexor policis brevis, the palmaris longus, the lumbricales, and the opponens policis; and a second group comprising (preferably consisting of): the triceps brachii (long head), the subscapularis, the pectoralis (e.g. the pectoralis major), the latissimus dorsi, the biceps brachii, the brachialis, the brachioradialis, and the teres major.

Preferably, when treating upper limb spasticity, modified BoNT/A may be administered to a plurality of affected muscles selected from: a first group comprising (preferably consisting of): the flexor digitorum superficialis, the flexor digitorum profundus, the flexor carpi radialis, the flexor carpi ulnaris, the brachioradialis, the pronator teres, and the biceps brachii; and a second group comprising (preferably consisting of): the triceps brachii (long head), the subscapularis, the pectoralis major, the latissimus dorsi, the biceps brachii, and the brachialis.

For example, preferably, when treating upper limb spasticity, modified BoNT/A may be administered to a plurality of affected muscles selected from: a first group comprising (preferably consisting of): the flexor digitorum superficialis, the flexor digitorum profundus, the flexor carpi radialis, the flexor carpi ulnaris, and the brachioradialis; and a second group comprising (preferably consisting of): the brachialis, the biceps brachii, the triceps brachii (long head), the subscapularis, the pectoralis (e.g. the pectoralis major), and the latissimus dorsi. More preferably, modified BoNT/A may be administered to a muscle associated with shoulder spasticity, such as adduced shoulder. Modified BoNT/A may be administered to at least one of the latissimus dorsi, the subscapularis, the pectoralis major, and the triceps brachii (long head). For example, modified BoNT/A may be administered to at least two or three (preferably all) of the latissimus dorsi, the subscapularis, the pectoralis major or the triceps brachii (long head).

When administering to the biceps brachii in some instances a single unit dose may be administered, while in other instances a 2× unit dose may be administered. Thus, the biceps brachii fall into both a first and second group of muscles. Preferably, 2× the unit dose is administered to the biceps brachii and this muscle is included in a second group of muscles.

Suitable dosages by upper limb muscle are shown below:

| Muscles Injected | Dosage |
|---|---|
| Flexor Digitorum Superficialis | 1x UD |
| (FDS) | |
| Flexor Digitorum Profundus | 1x UD |
| (FDP) | |
| Flexor Carpi Radialis (FCR) | 1x UD |
| Flexor Carpi Ulnaris (FCU) | 1x UD |
| Brachioradialis | 1x UD |
| Brachialis | 2x UD |
| Pronator Teres | 1x UD |
| Biceps Brachii | 2x UD |
| | 1x or 2x UD, |
| | (preferably 2x UD) |
| Triceps Brachii (long head) | 2x UD |
| Pectoralis Major | 2x UD |
| Subscapularis | 2x UD |
| Latissimus Dorsi | 2x UD |

Preferably, the modified BoNT/A may be administered to one or more of the muscles indicated at the dosages indicated in the table above. In some instances, the pronator teres may be omitted when treating limb spasticity.

Thus, preferably, suitable dosages by upper limb muscle are shown below:

| Muscles Injected | Dosage |
|---|---|
| Flexor Digitorum Superficialis (FDS) | 1x UD |
| Flexor Digitorum Profundus (FDP) | 1x UD |
| Flexor Carpi Radialis (FCR) | 1x UD |
| Flexor Carpi Ulnaris (FCU) | 1x UD |
| Brachioradialis | 1x UD |
| Brachialis | 2x UD |
| Biceps Brachii | 2x UD |
| Triceps Brachii (long head) | 2x UD |
| Pectoralis Major | 2x UD |
| Subscapularis | 2x UD |
| Latissimus Dorsi | 2x UD |

Modified BoNT/A may be administered to one or more of the following muscles as follows at the following dosages:

| Muscles Injected | Dosage |
|---|---|
| Deltoid | 1x UD |
| Levator scapulae | 1x UD |
| Pronator Quadratus | 1x UD |
| Pronator Teres | 1x UD |
| Brachioradialis | 1x UD |
| FCR | 1x UD |
| FCU | 1x UD |
| FDS | 1x UD |
| FDP | 1x UD |
| Flexor policis longus | 1x UD |
| Adductor policis | 1x UD |
| Flexor policis brevis | 1x UD |
| Palmaris longus | 1x UD |
| Lumbricales | 1x UD |
| Opponens policis | 1x UD |
| Latissimus Dorsi | 2x UD |
| Pectoralis | 2x UD |
| Brachioradialis | 2x UD |
| Teres Major | 2x UD |
| Subscapularis | 2x UD |
| Brachialis | 2x UD |
| Biceps Brachii | 2x UD |

To treat the upper limb spasticity clinical presentation “clenched fist”, the modified BoNT/A may be administered to the flexor digitorum superficialis and/or the flexor digitorum profundus. To treat the upper limb spasticity clinical presentation “flexed wrist”, the modified BoNT/A may be administered to the flexor carpi radialis and/or the flexor carpi ulnaris. To treat the upper limb spasticity clinical presentation “flexed elbow”, the modified BoNT/A may be administered to the brachioradialis, the brachialis, and/or the biceps brachii. To treat the upper limb spasticity clinical presentation “adducted shoulder”, the modified BoNT/A may be administered to the triceps brachii (long head), the subscapularis, the pectoralis major, and/or the latissimus dorsi.

Particularly preferably, during treatment of upper limb spasticity at least the clinical presentations “flexed elbow” and “adducted shoulder” are treated. More preferably, during treatment of upper limb spasticity the clinical presentations “flexed elbow” and “adducted shoulder” are treated and either (preferably only either) “clenched fist” or “flexed wrist” is treated.

The total number of unit doses administered in a given treatment may be up to 15× the unit dose. In other words, in one embodiment, 15× single unit doses may be administered at 15× injections sites. In another embodiment, 15× single unit doses may be administered at more than 15× injections sites. The total number of unit doses will be divided according to the muscles treated, e.g. 2× unit doses may be administered to the latissimus dorsi, 2× to the subscapularis, 2× to the pectoralis major, and 1× to the flexor carpi ulnaris yielding a total multiple of unit doses administered of 7×. For example, the total number of unit doses administered may be up to 14×, 13×, 12×, 11×, 10×, 9×, 8× or 7×. The total number of unit doses administered may be at least 2×, 3×, 4×, 5×, 6×, 7× the unit dose, preferably at least 2×. The total number of unit doses administered may be 2× to 15×, 7× to 15× or 10× to 14×. Preferably, the number of unit doses administered is 15×.

The skilled person will take into consideration when a subject has recently had (or is subsequently having) additional treatment with a clostridial neurotoxin (e.g. unmodified BoNT), e.g. as part of a cosmetic treatment or treatment for a different indication. Using techniques routine in the art, the skilled person will adapt the present treatment regimen accordingly.

It is preferred that the limb spasticity for treatment in accordance with the present invention is adult limb spasticity. Preferably, the dosage details provided above are for treating adult limb spasticity (unless context indicates that the dosage details provided are for treating paediatric limb spasticity). However, the treatment of paediatric limb spasticity is also encompassed. When treating paediatric limb spasticity, the combined total dosage is typically 50% or less than that used when treating adult limb spasticity. In some embodiments, the combined total dosage is typically 70% or less (e.g. 67% or less) than that used when treating adult limb spasticity.

The total number of unit doses administered in a given treatment for treating paediatric limb spasticity may be up to 10× the unit dose. In other words, in one embodiment, 10× single unit doses may be administered at 10× injections sites. However, the total number of unit doses administered in a given treatment for treating paediatric limb spasticity may be up to 15× the unit dose. In another embodiment, 10× single unit doses may be administered at more than 10× injections sites. The total number of unit doses will be divided according to the muscles treated, e.g. 2× unit doses may be administered to the latissimus dorsi, 2× to the subscapularis, 2× to the pectoralis major, and 1× to the flexor carpi ulnaris yielding a total multiple of unit doses administered of 7×. For example, the total number of unit doses administered may be up to 9×, 8×, 7×, 6×, 5×, 4× or 3×. The total number of unit doses administered may be at least 2×, 3×, 4×, 5×, 6×, 7× the unit dose, preferably at least 2×. The total number of unit doses administered may be 2× to 10×, 7× to 10× or 4× to 8×. Preferably, the number of unit doses administered is 10×. Preferably, the number of unit doses administered is 15×.

A modified BoNT/A of the invention preferably has a longer duration of action (e.g. an improvement in one or more symptoms of at least 5%, 10%, 25%, or 50%) when compared to unmodified BoNT/A (e.g. Dysport®). Said duration of action may be at least 1.25×, 1.5×, 1.75×, 2.0×, or 2.25× greater. The duration of action of modified BoNT/A may be between 6 and 9 months. For example, a duration of action may be at least: 4.5 months (from onset), 5.0 months, 5.5 months, 6 months, 6.5 months, 7.0 months, 7.5 months, 8.0 months, 8.5 months or 9.0 months. In particular embodiments, a duration of action may be greater than 9.0 months.

Administration to the plurality of muscles in accordance with the present invention preferably occurs in the same treatment session.

Treatment may be repeated at an appropriate time period following administration of modified BoNT/A. Given that the duration of action is approximately twice that of unmodified BoNT/A (e.g. Dysport®) there are suitably longer periods between subsequent administrations than when a subject is treated with unmodified BoNT/A (e.g. Dysport®). A subject may be re-administered a modified BoNT/A in accordance with the present invention at least 18, 20, 25 or 30 weeks following a previous administration. For example, a subject may be re-administered a modified BoNT/A in accordance with the present invention at least 18-45 weeks, preferably 20-35 weeks following a previous administration.

A “subject” as used herein may be a mammal, such as a human or other mammal. Preferably “subject” means a human subject. A “subject” is preferably an adult subject, i.e. a subject at least 18 years old. The terms “subject” and “patient” are used synonymously herein. When treating paediatric limb spasticity, the subject is less than 18 years old, e.g. less than 15, 10 or 5 years, such as 1-17, 5-17 or 10-17 years old. Preferably, the subject has been diagnosed with limb spasticity. The subject may have been diagnosed with spastic hemiparesis. The limb spasticity may have arisen following (e.g. been caused by) stroke or traumatic brain injury.

The skilled person will appreciate that where the limb spasticity is adult limb spasticity, the subject is an adult subject and where the limb spasticity is paediatric limb spasticity, the subject is a paediatric subject.

A subject for treatment in accordance with the invention may be a subject that is unsuitable for treatment with an unmodified BoNT/A and/or with another clostridial neurotoxin. Said subject may be a subject that is resistant to treatment with an unmodified BoNT/A and/or with another clostridial neurotoxin. Resistance may arise due to development of an immune response to a clostridial neurotoxin, including production of anti-clostridial neurotoxin antibodies, by a subject.

The dosages (e.g. unit doses and total dosages) described herein are for adult limb spasticity unless otherwise indicated.

The term “treat” or “treating” as used herein encompasses prophylactic treatment (e.g. to prevent onset of a disorder) as well as corrective treatment (treatment of a subject already suffering from a disorder). Preferably “treat” or “treating” as used herein means corrective treatment. The term “treat” or “treating” as used herein refers to the disorder and/or a symptom thereof.

Suitable modified BoNT/A polypeptides (and nucleotide sequences encoding the same, where present) are described in WO 2015/004461 A1 and WO 2017/191315, both of which are incorporated herein by reference in their entirety.

BoNT/A is one example of a clostridial neurotoxin produced by bacteria in the genus Clostridia. Other examples of such clostridial neurotoxins include those produced by C. tetani (TeNT) and by C. botulinum (BoNT) serotypes B-G, and X (see WO 2018/009903 A2), as well as those produced by C. baratii and C. butyricum. Said neurotoxins are highly potent and specific and can poison neurons and other cells to which they are delivered. The clostridial toxins are some of the most potent toxins known. Byway of example, botulinum neurotoxins have median lethal dose (LD₅₀) values for mice ranging from 0.5 to 5 ng/kg, depending on the serotype. Both tetanus and botulinum toxins act by inhibiting the function of affected neurons, specifically the release of neurotransmitters. While botulinum toxin acts at the neuromuscular junction and inhibits cholinergic transmission in the peripheral nervous system, tetanus toxin acts in the central nervous system.

In nature, clostridial neurotoxins (including BoNT/A) are synthesised as a single-chain polypeptide that is modified post-translationally by a proteolytic cleavage event to form two polypeptide chains joined together by a disulphide bond. Cleavage occurs at a specific cleavage site, often referred to as the activation site (e.g. activation loop), that is located between the cysteine residues that provide the inter-chain disulphide bond. It is this di-chain form that is the active form of the toxin. The two chains are termed the heavy chain (H-chain), which has a molecular mass of approximately 100 kDa, and the light chain (L-chain), which has a molecular mass of approximately 50 kDa. The H-chain comprises an N-terminal translocation component (H_{N} domain) and a C-terminal targeting component (H_{C} domain). The cleavage site is located between the L-chain and the translocation domain components. Following binding of the H_{C} domain to its target neuron and internalisation of the bound toxin into the cell via an endosome, the H_{N} domain translocates the L-chain across the endosomal membrane and into the cytosol, and the L-chain provides a protease function (also known as a non-cytotoxic protease).

Non-cytotoxic proteases act by proteolytically cleaving intracellular transport proteins known as SNARE proteins (e.g. SNAP-25, VAMP, or Syntaxin)—see Gerald K (2002) “Cell and Molecular Biology” (4th edition) John Wiley & Sons, Inc, preferably SNAP-25. The acronym SNARE derives from the term Soluble NSF Attachment Receptor, where NSF means N-ethylmaleimide-Sensitive Factor. SNARE proteins are integral to intracellular vesicle fusion, and thus to secretion of molecules via vesicle transport from a cell. The protease function is a zinc-dependent endopeptidase activity and exhibits a high substrate specificity for SNARE proteins. Accordingly, once delivered to a desired target cell, the non-cytotoxic protease is capable of inhibiting cellular secretion from the target cell. The L-chain proteases of clostridial neurotoxins are non-cytotoxic proteases that cleave SNARE proteins.

The term “H_{C} domain” as used herein means a functionally distinct region of a neurotoxin heavy chain with a molecular weight of approximately 50 kDa that enables the binding of the neurotoxin to a receptor located on the surface of the target cell. The H_{C} domain consists of two structurally distinct subdomains, the “H_{CN} subdomain” (N-terminal part of the H_{C} domain) and the “H_{CC} subdomain” (C-terminal part of the H_{C} domain), each of which has a molecular weight of approximately 25 kDa.

The term “LH_{N} domain” as used herein means a neurotoxin that is devoid of the H_{C} domain and consists of an endopeptidase domain (“L” or “light chain”) and the domain responsible for translocation of the endopeptidase into the cytoplasm (H_{N} domain of the heavy chain).

In view of the ubiquitous nature of SNARE proteins, clostridial neurotoxins such as botulinum toxin have been successfully employed in a wide range of therapies.

For further details on the genetic basis of toxin production in Clostridium botulinum and C. tetani, see Henderson et al (1997) in The Clostridia: Molecular Biology and Pathogenesis, Academic press.

As discussed above, clostridial neurotoxins are formed from two polypeptide chains, the heavy chain (H-chain), which has a molecular mass of approximately 100 kDa, and the light chain (L-chain), which has a molecular mass of approximately 50 kDa. The H-chain comprises a C-terminal targeting component (receptor binding domain or H_{C} domain) and an N-terminal translocation component (H_{N} domain).

Clostridial neurotoxin domains are described in more detail below.

Examples of light chain reference sequences include:
Botulinum type A neurotoxin: amino acid residues 1-448
Botulinum type B neurotoxin: amino acid residues 1-440

The above-identified reference sequences should be considered a guide, as slight variations may occur according to sub-serotypes. By way of example, US 2007/0166332 (hereby incorporated by reference in its entirety) cites slightly different clostridial sequences:
Botulinum type A neurotoxin: amino acid residues M1-K448
Botulinum type B neurotoxin: amino acid residues M1-K441

The translocation domain is a fragment of the H-chain of a clostridial neurotoxin approximately equivalent to the amino-terminal half of the H-chain, or the domain corresponding to that fragment in the intact H-chain.

Examples of reference translocation domains include:
Botulinum type A neurotoxin-amino acid residues (449-871)
Botulinum type B neurotoxin-amino acid residues (441-858)

The above-identified reference sequence should be considered a guide as slight variations may occur according to sub-serotypes. By way of example, US 2007/0166332 (hereby incorporated by reference thereto) cites slightly different clostridial sequences:
Botulinum type A neurotoxin-amino acid residues (A449-K871)
Botulinum type B neurotoxin-amino acid residues (A442-S858)

In the context of the present invention, a variety of BoNT/A H_{N} regions comprising a translocation domain can be useful in aspects of the present invention. The H_{N} regions from the heavy-chain of BoNT/A are approximately 410-430 amino acids in length and comprise a translocation domain. Research has shown that the entire length of a H_{N} region from a clostridial neurotoxin heavy-chain is not necessary for the translocating activity of the translocation domain. Thus, aspects of this embodiment can include BoNT/A H_{N} regions comprising a translocation domain having a length of, for example, at least 350 amino acids, at least 375 amino acids, at least 400 amino acids or at least 425 amino acids. Other aspects of this embodiment can include BoNT/A H_{N} regions comprising a translocation domain having a length of, for example, at most 350 amino acids, at most 375 amino acids, at most 400 amino acids or at most 425 amino acids.

The term H_{N} embraces naturally-occurring BoNT/A H_{N} portions, and modified BoNT/A H_{N} portions having amino acid sequences that do not occur in nature and/or synthetic amino acid residues. Preferably, said modified BoNT/A H_{N} portions still demonstrate the above-mentioned translocation function.

Examples of clostridial neurotoxin receptor binding domain (H_{C}) reference sequences include:
BoNT/A—N872-L1296
BoNT/B—E859-E1291

The ˜50 kDa H_{C} domain of a clostridial neurotoxin (such as a BoNT) comprises two distinct structural features that are referred to as the H_{CC} and H_{CN} domains, each typically of ˜25 kDa. Amino acid residues involved in receptor binding are believed to be primarily located in the H_{CC} domain. The H_{C} domain of a native clostridial neurotoxin may comprise approximately 400-440 amino acid residues. This fact is confirmed by the following publications, each of which is herein incorporated in its entirety by reference thereto: Umland T C (1997) Nat. Struct. Biol. 4: 788-792; Herreros J (2000) Biochem. J. 347: 199-204; Halpern J (1993) J. Biol. Chem. 268: 15, pp. 11188-11192; Rummel A (2007) PNAS 104: 359-364; Lacey D B (1998) Nat. Struct. Biol. 5: 898-902; Knapp (1998) Am. Cryst. Assoc. Abstract Papers 25: 90; Swaminathan and Eswaramoorthy (2000) Nat. Struct. Biol. 7: 1751-1759; and Rummel A (2004) Mol. Microbiol. 51(3), 631-643.

Examples of (reference) H_{CN} domains include:
Botulinum type A neurotoxin-amino acid residues (872-1110)
Botulinum type B neurotoxin-amino acid residues (859-1097)

The above sequence positions may vary a little according to serotype/sub-type, and further examples of (reference) H_{CN} domains include:
Botulinum type A neurotoxin-amino acid residues (874-1110)
Botulinum type B neurotoxin-amino acid residues (861-1097)

Examples of (reference) H_{CC} domains include:
Botulinum type A neurotoxin-amino acid residues (Y1111-L1296)
Botulinum type B neurotoxin-amino acid residues (Y1098-E1291)

WO 2017/191315 A1 (which is incorporated herein by reference) teaches modified BoNT/As and methods for preparing and manufacturing the same. Thus, a modified BoNT/A comprising a botulinum neurotoxin A (BoNT/A) light-chain and translocation domain (BoNT/A H_{N}), and a BoNT/B receptor binding domain (H_{C} domain) for use in the present invention may be one taught in WO 2017/191315 A1.

The term “modified BoNT/A” or “chimeric neurotoxin” as used herein means a neurotoxin comprising (preferably consisting of) a clostridial neurotoxin light-chain and translocation domain (H_{N} domain) from a first clostridial neurotoxin serotype and a receptor binding domain (H_{C} domain) originating from a second different clostridial neurotoxin serotype. Specifically, a modified BoNT/A for use in the invention comprises a botulinum neurotoxin A (BoNT/A) light-chain and translocation domain (H_{N} domain), and a BoNT/B receptor binding domain (H_{C} domain). The BoNT/A LH_{N} domain of the modified BoNT/A is covalently linked to the BoNT/B H_{C} domain. The modified BoNT/A of the invention may be referred to as a chimeric botulinum neurotoxin. Said modified BoNT/A is also referred to herein as “BoNT/AB”, “mrBoNT/AB” or a “BoNT/AB chimera”.

The L-chain and H_{N} domain (optionally including a complete or partial activation loop, e.g. a complete activation loop when the modified BoNT/A is in a single-chain form and a cleaved/partial activation loop when in a di-chain form) may be collectively referred to as an LH_{N} domain. The LH_{N} domain thus does not further comprise an H_{C} domain.

The modified BoNT/A may consist essentially of a botulinum neurotoxin A (BoNT/A) light-chain and translocation domain (H_{N} domain), and a BoNT/B receptor binding domain (H_{C} domain). The term “consist(s) essentially of” as used in this context means that the modified BoNT/A does not further comprise one or more amino acid residues that confer additional functionality to the polypeptide, e.g. when administered to a subject. In other words, a polypeptide that “consists essentially of” a botulinum neurotoxin A (BoNT/A) light-chain and translocation domain (H_{N} domain), and a BoNT/B receptor binding domain (H_{C} domain) may further comprise one or more amino acid residues (to those of the botulinum neurotoxin A (BoNT/A) light-chain and translocation domain (H_{N} domain), and BoNT/B receptor binding domain (H_{C} domain)) but said one or more further amino acid residues do not confer additional functionality to the polypeptide, e.g. when administered to a subject. Additional functionality may include enzymatic activity, binding activity and/or any physiological activity whatsoever.

The modified BoNT/A may comprise non-clostridial neurotoxin sequences in addition to any clostridial neurotoxin sequences so long as the non-clostridial neurotoxin sequences do not disrupt the ability of the modified BoNT/A to achieve its therapeutic effect. Preferably, the non-clostridial neurotoxin sequence is not one having catalytic activity, e.g. enzymatic activity. In one embodiment the modified BoNT/A of the invention does not comprise a non-clostridial catalytically active domain. In one embodiment, a modified BoNT/A does not comprise a further catalytically active domain. In one embodiment, the non-clostridial sequence is not one that binds to a cellular receptor. In other words, in one embodiment, the non-clostridial sequence is not a ligand for a cellular receptor. A cellular receptor may be a proteinaceous cellular receptor, such as an integral membrane protein. Examples of cellular receptors can be found in the IUPHAR Guide to Pharmacology Database, version 2019.4, available at https://www.guidetopharmacology.org/download.jsp#db_reports. Non-clostridial neurotoxin sequences may include tags to aid in purification, such as His-tags. In one embodiment, a modified BoNT/A of the invention does not comprise a label or a site for adding a label, such as a sortase acceptor or donor site.

Preferably, a modified BoNT/A may consist of a botulinum neurotoxin A (BoNT/A) light-chain and translocation domain (H_{N} domain), and a BoNT/B receptor binding domain (H_{C} domain).

The modified BoNT/A comprises a light-chain that is capable of exhibiting non-cytotoxic protease activity and of cleaving a SNARE protein in the cytosol of a target neuron.

Cell-based and in vivo assays may be used to determine if a clostridial neurotoxin comprising an L-chain and a functional cell binding and translocation domain has non-cytotoxic protease activity. Assays such as the Digit Abduction Score (DAS) assay, the dorsal root ganglia (DRG) assay, spinal cord neuron (SCN) assay, and mouse phrenic nerve hemidiaphragm (PNHD) assay are routine in the art. A suitable assay for determining non-cytotoxic protease activity may be one described in Aoki K R, Toxicon 39: 1815-1820; 2001 or Donald et al (2018), Pharmacol Res Perspect, e00446, 1-14, which are incorporated herein by reference.

When administered to a subject, a modified BoNT/A is preferably in its active di-chain form where the light-chain and heavy-chain are joined together by a disulphide bond. Where a BoNT/A (e.g. modified BoNT/A) is defined herein by way of a polypeptide sequence (SEQ ID NO), an L-chain portion of the sequence (SEQ ID NO) may constitute a first chain of the di-chain clostridial neurotoxin (e.g. di-chain modified BoNT/A) and the H_{N} and H_{C} domains together may constitute a second chain of the di-chain clostridial neurotoxin (e.g. di-chain modified BoNT/A), wherein the first and second chains are joined together by a di-sulphide bond. The skilled person will appreciate that a protease may cleave at one or more positions within the activation loop of the clostridial neurotoxin (e.g. modified BoNT/A), preferably at two positions within the activation loop. Where cleavage occurs at more than one position (preferably at two positions) within the activation loop, a small fragment of the C-terminal L-chain portion of the sequence may be absent from the di-chain clostridial neurotoxin sequence (e.g. di-chain modified BoNT/A). In view of this, the sequence of the di-chain clostridial neurotoxin (e.g. di-chain modified BoNT/A) may be slightly different to that of the corresponding single-chain clostridial neurotoxin (e.g. single-chain modified BoNT/A). The small fragment may be 1-15 amino acids. In particular, in one embodiment, when Lys-C is used to covert a single-chain modified BoNT/A into a di-chain modified BoNT/A, the small fragment of the C-terminal L-chain portion of the sequence that is absent may be SEQ ID NO: 9 or 10.

Most preferably, a modified BoNT/A for use in the invention may comprise a BoNT/A light-chain and translocation domain (a BoNT/A LH_{N} domain), and a BoNT/B H_{C} domain. The BoNT/A LH_{N} domain is covalently linked to the BoNT/B H_{C} domain. Said modified BoNT/A is also referred to herein as “BoNT/AB” or a “BoNT/AB chimera”.

The C-terminal amino acid residue of the LH_{N} domain may correspond to the first amino acid residue of the 3₁₀ helix separating the LH_{N} and H_{C} domains of BoNT/A, and the N-terminal amino acid residue of the H_{C} domain may correspond to the second amino acid residue of the 3₁₀ helix separating the LH_{N} and H_{C} domains in BoNT/B.

An example of a (unmodified) BoNT/A polypeptide sequence is provided as SEQ ID NO: 2.

An example of a BoNT/B polypeptide sequence is provided as SEQ ID NO: 8 (UniProt accession number B1INP5).

Reference herein to the “first amino acid residue of the 3₁₀ helix separating the LH_{N} and He domains of BoNT/A” means the N-terminal residue of the 3₁₀ helix separating the LH_{N} and He domains.

Reference herein to the “second amino acid residue of the 3₁₀ helix separating the LH_{N} and He domains of BoNT/B” means the amino acid residue following the N-terminal residue of the 3₁₀ helix separating the LH_{N} and H_{C} domains.

A “3₁₀ helix” is a type of secondary structure found in proteins and polypeptides, along with α-helices, β-sheets and reverse turns. The amino acids in a 3₁₀ helix are arranged in a right-handed helical structure where each full turn is completed by three residues and ten atoms that separate the intramolecular hydrogen bond between them. Each amino acid corresponds to a 120° turn in the helix (i.e., the helix has three residues per turn), and a translation of 2.0 Å (=0.2 nm) along the helical axis, and has 10 atoms in the ring formed by making the hydrogen bond. Most importantly, the N—H group of an amino acid forms a hydrogen bond with the C═O group of the amino acid three residues earlier; this repeated i+3→i hydrogen bonding defines a 3₁₀ helix. A 3₁₀ helix is a standard concept in structural biology with which the skilled person is familiar.

This 3₁₀ helix corresponds to four residues which form the actual helix and two cap (or transitional) residues, one at each end of these four residues. The term “3₁₀ helix separating the LH_{N} and H_{C} domains” as used herein consists of those 6 residues.

Through carrying out structural analyses and sequence alignments, a 3₁₀ helix separating the LH_{N} and H_{C} domains was identified. This 3₁₀ helix is surrounded by an α-helix at its N-terminus (i.e. at the C-terminal part of the LH_{N} domain) and by a β-strand at its C-terminus (i.e. at the N-terminal part of the H_{C} domain). The first (N-terminal) residue (cap or transitional residue) of the 3₁₀ helix also corresponds to the C-terminal residue of this α-helix.

The 3₁₀ helix separating the LH_{N} and H_{C} domains can be for example determined from publicly available crystal structures of botulinum neurotoxins, for example 3BTA (http://www.rcsb.org/pdb/explore/explore.do?structureld=3BTA) and 1 EPW (http://www.rcsb.org/pdb/explore/explore.do?structureld=1EPW) for botulinum neurotoxins A1 and 1 respectively.

In silico modelling and alignment tools which are publicly available can also be used to determine the location of the 3₁₀ helix separating the LH_{N} and H_{C} domains in other neurotoxins, for example the homology modelling servers LOOPP (Learning, Observing and Outputting Protein Patterns, http://loopp.org), PHYRE (Protein Homology/analogY Recognition Engine, http://www.sbg.bio.ic.ac.uk/phyre2/) and Rosetta (https://www.rosettacommons.org/), the protein superposition server SuperPose (http://wishart.biology.ualberta.ca/superpose/), the alignment program Clustal Omega (http://www.clustal.org/omega/), and a number of other tools/services listed at the Internet Resources for Molecular and Cell Biologists (http://molbiol-tools.ca/). In particular the region around the “H_{N}/H_{CN}” junction may be structurally highly conserved which renders it an ideal region to superimpose different serotypes.

For example, the following methodology may be used to determine the sequence of this 3₁₀ helix in other neurotoxins:
1. The structural homology modelling tool LOOP (http://loopp.org) may be used to obtain a predicted structure of other BoNT serotypes based on the BoNT/A1 crystal structure (3BTA.pdb);
2. The structural (pdb) files thus obtained may be edited to include only the N-terminal end of the H_{CN} domain and about 80 residues before it (which are part of the H_{N} domain), thereby retaining the “H_{N}/H_{CN}” region which is structurally highly conserved;
3. The protein superposition server SuperPose (http://wishart.biology.ualberta.ca/superpose) may be used to superpose each serotype onto the 3BTA.pdb structure;
4. The superposed pdb files were inspected to locate the 3₁₀ helix at the start of the H_{C} domain of BoNT/A1, and corresponding residues in the other serotype may then be identified.
5. The other BoNT serotype sequences may be aligned with Clustal Omega in order to check that corresponding residues were correct.

Examples of LH_{N}, H_{C} and 3₁₀ helix domains determined by this method are presented below:

| | Accession Number | | | |
|---|---|---|---|---|
| | (Plus Sequence | | | |
| | Version after | | | |
| Neurotoxin | Decimal) | LH_{N} | H_{C} | 3₁₀ helix |
| BoNT/A1 | A5HZZ9.1 | 1-872 | 873-1296 | ⁸⁷²NIINTS⁸⁷⁷ |
| (SEQ ID | | | | |
| NO: 2) | | | | |
| BoNT/A2 | X73423.3 | 1-872 | 873-1296 | ⁸⁷²NIVNTS⁸⁷⁷ |
| BoNT/A3 | DQ185900.1 (aka | 1-872 | 873-1292 | ⁸⁷²NIVNTS⁸⁷⁷ |
| | Q3LRX9.1) | | | |
| BoNT/A4 | EU341307.1 (aka | 1-872 | 873-1296 | ⁸⁷²NITNAS⁸⁷⁷ |
| | Q3LRX8.1) | | | |
| BoNT/A5 | EU679004.1 (aka | 1-872 | 873-1296 | ⁸⁷²NIINTS⁸⁷⁷ |
| | C1IPK2.1) | | | |
| BoNT/A6 | FJ981696.1 | 1-872 | 873-1296 | ⁸⁷²NIINTS⁸⁷⁷ |
| BoNT/A7 | JQ954969.1 (aka | 1-872 | 873-1296 | ⁸⁷²NIINTS⁸⁷⁷ |
| | K4LN57.1) | | | |
| BoNT/A8 | KM233166.1 | 1-872 | 873-1297 | ⁸⁷²NITNTS⁸⁷⁷ |
| BoNT/B1 | B1INP5.1 | 1-859 | 860-1291 | ⁸⁵⁹EILNNI⁸⁶⁴ |
| (SEQ ID | | | | |
| NO: 8) | | | | |
| BoNT/B2 | AB084152.1 (aka | 1-859 | 860-1291 | ⁸⁵⁹EILNNI⁸⁶⁴ |
| | Q8GR96.1) | | | |
| BoNT/B3 | EF028400.1 (aka | 1-859 | 860-1291 | ⁸⁵⁹EILNNI⁸⁶⁴ |
| | A2I2S2.1) | | | |
| BoNT/B4 | EF051570.1 (aka | 1-859 | 860-1291 | ⁸⁵⁹EILNNI⁸⁶⁴ |
| | A2I2W0.1) | | | |
| BoNT/B5 | EF033130.1 (aka | 1-859 | 860-1291 | ⁸⁵⁹DILNNI⁸⁶⁴ |
| | A2I2U6.1) | | | |
| BoNT/B6 | AB302852.1 (aka | 1-859 | 860-1291 | ⁸⁵⁹EILNNI⁸⁶⁴ |
| | A8R089.1) | | | |
| BoNT/B7 | JQ354985.1 (aka | 1-859 | 860-1291 | ⁸⁵⁹EILNNI⁸⁶⁴ |
| | H9CNK9.1) | | | |
| BoNT/B8 | JQ964806.1 (aka | 1-859 | 860-1292 | ⁸⁵⁹EILNNI⁸⁶⁴ |
| | I6Z8G9.1) | | | |

Using structural analysis and sequence alignments, it was found that the β-strand following the 3₁₀ helix separating the LH_{N} and H_{C} domains is a conserved structure in all botulinum and tetanus neurotoxins and starts at the 8^{th} residue when starting from the first residue of the 3₁₀ helix separating the LH_{N} and H_{C} domains (e.g., at residue 879 for BoNT/A1).

A BoNT/AB chimera may comprise an LH_{N} domain from BoNT/A covalently linked to a He domain from BoNT/B,
wherein the C-terminal amino acid residue of the LH_{N} domain corresponds to the eighth amino acid residue N-terminally to the β-strand located at the beginning (N-term) of the H_{C} domain of BoNT/A, and
wherein the N-terminal amino acid residue of the H_{C} domain corresponds to the seventh amino acid residue N-terminally to the β-strand located at the beginning (N-term) of the H_{C} domain of BoNT/B.

A BoNT/AB chimera may comprise an LH_{N} domain from BoNT/A covalently linked to a He domain from BoNT/B,
wherein the C-terminal amino acid residue of the LH_{N} domain corresponds to the C-terminal amino acid residue of the α-helix located at the end (C-term) of LH_{N} domain of BoNT/A, and
wherein the N-terminal amino acid residue of the H_{C} domain corresponds to the amino acid residue immediately C-terminal to the C-terminal amino acid residue of the α-helix located at the end (C-term) of LH_{N} domain of BoNT/B.

The rationale of the design process of the BoNT/AB chimera was to try to ensure that the secondary structure was not compromised and thereby minimise any changes to the tertiary structure and to the function of each domain. Without wishing to be bound by theory, it is hypothesized that by not disrupting the four central amino acid residues of the 3₁₀ helix in the BoNT/AB chimera ensures an optimal conformation for the chimeric neurotoxin, thereby allowing for the chimeric neurotoxin to exert its functions to their full capacity.

In fact, surprisingly, retaining solely the first amino acid residue of the 3₁₀ helix of the BoNT/A and the second amino acid residue of the 3₁₀ helix onwards of BoNT/B not only allows the production of soluble and functional BoNT/AB chimera, but further leads to improved properties over other BoNT/AB chimeras, in particular an increased potency, an increased Safety Ratio and/or a longer duration of action (as well as increased Safety Ratio and/or duration of action when compared to unmodified BoNT/A [e.g. SEQ ID NO: 2, such as SEQ ID NO: 2 in a di-chain form]).

The BoNT/A light-chain, BoNT/A translocation domain, and/or BoNT/B H_{C} domain may be a modified BoNT/A light-chain, BoNT/A translocation domain, and/or BoNT/B H_{C} domain or a derivative thereof, including but not limited to those described below. A modified BoNT/A light-chain, BoNT/A translocation domain, and/or BoNT/B H_{C} domain or derivative may contain one or more amino acids that has been modified as compared to the native (unmodified) form of the BoNT/A light-chain, BoNT/A translocation domain, and/or BoNT/B H_{C} domain, or may contain one or more inserted amino acids that are not present in the native (unmodified) form of the BoNT/A light-chain, BoNT/A translocation domain, and/or BoNT/B H_{C} domain. By way of example, a modified BoNT/A light-chain, BoNT/A translocation domain, and/or BoNT/B He domain may have modified amino acid sequences in one or more domains relative to the native (unmodified) BoNT/A light-chain, BoNT/A translocation domain, and/or BoNT/B H_{C} domain sequence. Such modifications may modify functional aspects thereof, for example biological activity or persistence. Thus, in one embodiment, the BoNT/A light-chain, BoNT/A translocation domain, and/or BoNT/B H_{C} domain is a modified BoNT/A light-chain, BoNT/A translocation domain, and/or BoNT/B H_{C} domain, or modified BoNT/A light-chain, BoNT/A translocation domain, and/or BoNT/B H_{C} domain derivative.

A modified BoNT/B H_{C} domain may have one or more modifications modifying binding to target nerve cells, for example providing higher or lower affinity binding when compared to the native (unmodified) BoNT/B H_{C} domain. Such modifications in the BoNT/B H_{C} domain may include modifying residues in the ganglioside binding site of the H_{C} domain or in the protein (e.g. synaptotagmin) binding site that alter binding to the ganglioside receptor and/or the protein receptor of the target nerve cell. Examples of such modified neurotoxins are described in WO 2006/027207 and WO 2006/114308, both of which are hereby incorporated by reference in their entirety.

A modified light-chain may have one or more modifications in the amino acid sequence thereof, for example modifications in the substrate binding or catalytic domain which may alter or modify the SNARE protein specificity of the modified light-chain, preferably with the proviso that said modifications do not catalytically inactivate said light-chain. Examples of such modified neurotoxins are described in WO 2010/120766 and US 2011/0318385, both of which are hereby incorporated by reference in their entirety.

The LH_{N} domain from BoNT/A may correspond to amino acid residues 1 to 872 of SEQ ID NO: 2, or a polypeptide sequence having at least 70% sequence identity thereto. The LH_{N} domain from BoNT/A may correspond to amino acid residues 1 to 872 of SEQ ID NO: 2, or a polypeptide sequence having at least 80%, 90% or 95% sequence identity thereto. Preferably, the LH_{N} domain from BoNT/A corresponds to amino acid residues 1 to 872 of SEQ ID NO: 2.

The H_{C} domain from BoNT/B may correspond to amino acid residues 860 to 1291 of SEQ ID NO: 8 or a polypeptide sequence having at least 70% sequence identity thereto. The He domain from BoNT/B may correspond to amino acid residues 860 to 1291 of SEQ ID NO: 8, or a polypeptide sequence having at least 80%, 90% or 95% sequence identity thereto. Preferably, the H_{C} domain from BoNT/B corresponds to amino acid residues 860 to 1291 of SEQ ID NO: 8.

Preferably, the BoNT/AB chimera comprises a BoNT/A1 LH_{N} domain and a BoNT/B1 He domain. More preferably, the LH_{N} domain corresponds to amino acid residues 1 to 872 of BoNT/A1 (SEQ ID NO: 2) and the H_{C} domain corresponds to amino acid residues 860 to 1291 of BoNT/B1 (SEQ ID NO: 8).

Most preferably, a BoNT/B H_{C} domain further comprises at least one amino acid residue substitution, insertion, indel or deletion in the H_{CC} subdomain which has the effect of increasing the binding affinity of BoNT/B neurotoxin for human Syt II as compared to the natural BoNT/B sequence. Suitable amino acid residue substitutions, insertions, indels or deletions in the BoNT/B H_{CC} subdomain have been disclosed in WO 2013/180799 and in WO 2016/154534 (both herein incorporated by reference).

A suitable amino acid residue substitution, insertion, indel or deletion in the BoNT/B H_{CC} subdomain may include substitution mutations selected from the group consisting of: V1118M; Y1183M; E1191M; E11911; E1191Q; E1191T; S1199Y; S1199F; S1199L; S1201V; E1191C, E1191V, E1191L, E1191Y, S1199W, S1199E, S1199H, W1178Y, W1178Q, W1178A, W1178S, Y1183C, Y1183P and combinations thereof.

A suitable amino acid residue substitution, insertion, indel or deletion in the BoNT/B H_{CC} subdomain may further include combinations of two substitution mutations selected from the group consisting of: E1191M and S1199L, E1191M and S1199Y, E1191M and S1199F, E1191Q and S1199L, E1191Q and S1199Y, E1191Q and S1199F, E1191M and S1199W, E1191M and W1178Q, E1191C and S1199W, E1191C and S1199Y, E1191C and W1178Q, E1191Q and S1199W, E1191V and S1199W, E1191V and S1199Y, or E1191V and W1178Q.

A suitable amino acid residue substitution, insertion, indel or deletion in the BoNT/B H_{CC} subdomain may also include a combination of three substitution mutations which are E1191M, S1199W and W1178Q.

Preferably, the amino acid residue substitution, insertion, indel or deletion in the BoNT/B H_{CC} subdomain includes a combination of two substitution mutations which are E1191M and S1199Y. Such modifications are present in modified BoNT/As SEQ ID NO: 5 and SEQ ID NO: 6. E1191M may correspond to position 1204 of SEQ ID NO: 6 and S1199Y may correspond to position 1212. Thus, SEQ ID NO: 6 may comprise 1204M and 1212Y.

The modification may be a modification when compared to unmodified BoNT/B shown as SEQ ID NO: 8, wherein the amino acid residue numbering is determined by alignment with SEQ ID NO: 8. As the presence of a methionine residue at position 1 of SEQ ID NO: 8 (as well as the SEQ ID NOs corresponding to modified BoNT/A polypeptides described herein) is optional, the skilled person will take the presence/absence of the methionine residue into account when determining amino acid residue numbering. For example, where SEQ ID NO: 8 includes a methionine, the position numbering will be as defined above (e.g. E1191 will be E1191 of SEQ ID NO: 8). Alternatively, where the methionine is absent from SEQ ID NO: 8 the amino acid residue numbering should be modified by −1 (e.g. E1191 will be E1190 of SEQ ID NO: 8). Accordingly, an initial methionine amino acid residue of a polypeptide sequence of the modified BoNT/A may be optional or absent. Similar considerations apply when the methionine at position 1 of the other polypeptide sequences described herein is present/absent, and the skilled person will readily determine the correct amino acid residue numbering using techniques routine in the art.

A modified BoNT/A for use in the invention may comprise a polypeptide sequence having at least 70% sequence identity to a polypeptide sequence selected from SEQ ID NOs: 3-7. For example, a polypeptide sequence having at least 80%, 90%, 95% or 99.9% sequence identity to a polypeptide sequence selected from SEQ ID NOs: 3-7. Preferably, a modified BoNT/A for use in the invention may comprise (more preferably consist of) a polypeptide sequence selected from SEQ ID NOs: 3-7.

It is preferred that the modified BoNT/A comprises a polypeptide sequence having at least 70% sequence identity to SEQ ID NO: 6. For example, a polypeptide sequence having at least 80%, 90%, 95% or 99.9% sequence identity to SEQ ID NO: 6. Most preferably, a modified BoNT/A for use in the invention may comprise (more preferably consist of) SEQ ID NO: 6.

The term “deletion” as used herein refers to removal of one or more amino acid residues of a polypeptide without replacement of one or more amino acid residues at the site of deletion. Thus, where one amino acid residue has been deleted from a polypeptide sequence having x number of amino acid residues (for example), the resultant polypeptide has x−1 amino acid residues.

The term “indel” as used herein refers to deletion of one or more amino acid residues of a polypeptide and insertion at the deletion site of a different number of amino acid residues (either greater or fewer amino acid residues) when compared to the number of amino acid residues deleted. Thus, for an indel where two amino acid residues have been deleted from a polypeptide sequence having x number of amino acid residues (for example), the resultant polypeptide has x−1 amino acid residues or x+≥1 amino acid residues. The insertion and deletion can be carried out in any order, sequentially or simultaneously.

The term “substitution” as used herein refers to replacement of one or more amino acid residues with the same number of amino acid residues at the same site. Thus, for a substitution of a polypeptide sequence having x number of amino acid residues (for example), the resultant polypeptide also has x amino acid residues. Preferably a substitution is a substitution at a single amino acid position.

The term “insertion” as used herein refers to addition of one or more amino acid residues of a polypeptide without deletion of one or more amino acid residues of the polypeptide at the site of insertion. Thus, where one amino acid residue has been inserted into a polypeptide sequence having x number of amino acid residues (for example), the resultant polypeptide has x+1 amino acid residues.

Methods for modifying proteins by substitution, insertion or deletion of amino acid residues are known in the art. By way of example, amino acid modifications may be introduced by modification of a DNA sequence encoding a BoNT/A (e.g. encoding unmodified BoNT/A). This can be achieved using standard molecular cloning techniques, for example by site-directed mutagenesis where short strands of DNA (oligonucleotides) coding for the desired amino acid(s) are used to replace the original coding sequence using a polymerase enzyme, or by inserting/deleting parts of the gene with various enzymes (e.g., ligases and restriction endonucleases). Alternatively, a modified gene sequence can be chemically synthesised. Typically a modification may be carried out by either modifying a nucleic acid encoding a native clostridial neurotoxin (or part thereof) such that the modified BoNT/A (or part thereof) encoded by the nucleic acid comprises the modification(s). Alternatively, a nucleic acid that encodes a modified BoNT/A (or part thereof) comprising the modification(s) may be synthesized.

Where a polypeptide sequence of a modified BoNT/A described herein comprises a tag, e.g. for purification, such as a His-tag, said tag is optional. Preferably, said tag is removed prior to use of the modified BoNT/A according to the invention.

As discussed above, a modified BoNT/A described herein has increased tissue retention properties that also provide increased potency and/or duration of action and can allow for increased dosages without any additional negative effects. One way in which these advantageous properties may be defined is in terms of the Safety Ratio of the modified BoNT/A. In this regard, undesired effects of a clostridial toxin (caused by diffusion of the toxin away from the site of administration) can be assessed experimentally by measuring percentage bodyweight loss in a relevant animal model (e.g. a mouse, where loss of bodyweight is detected within seven days of administration). Conversely, desired on-target effects of a clostridial toxin can be assessed experimentally by Digital Abduction Score (DAS) assay, a measurement of muscle paralysis. The DAS assay may be performed by injection of 20 μl of neurotoxin, formulated in Gelatin Phosphate Buffer, into the mouse gastrocnemius/soleus complex, followed by assessment of Digital Abduction Score using the method of Aoki (Aoki K R, Toxicon 39: 1815-1820; 2001). In the DAS assay, mice are suspended briefly by the tail in order to elicit a characteristic startle response in which the mouse extends its hind limbs and abducts its hind digits. Following neurotoxin injection, the varying degrees of digit abduction are scored on a five-point scale (0=normal to 4=maximal reduction in digit abduction and leg extension).

The Safety Ratio of a neurotoxin may then be expressed as the ratio between the amount of toxin required for a 10% drop in a bodyweight (measured at peak effect within the first seven days after dosing in a mouse) and the amount of neurotoxin required for a DAS score of 2. High Safety Ratio scores are therefore desired and indicate a neurotoxin that is able to effectively paralyse a target muscle with little undesired off-target effects. A modified BoNT/A of the present invention has a Safety Ratio that is higher than the Safety Ratio of an equivalent unmodified (native) BoNT/A.

A high Safety Ratio is particularly advantageous in therapy because it represents an increase in the therapeutic index. In other words, this means that reduced dosages can be used compared to alternative clostridial neurotoxin therapeutics and/or that increased dosages can be used without any additional (e.g. deleterious) effects. Deleterious effects may include systemic toxicity and/or undesired spread to adjacent muscles. The possibility to use higher doses of neurotoxin without additional effects is particularly advantageous as higher doses usually lead to a longer duration of action of the neurotoxin.

The potency of a modified BoNT/A may be expressed as the minimal dose of neurotoxin which leads to a given DAS score when administered to a mouse gastrocnemius/soleus complex, for example a DAS score of 2 (ED₅₀ dose) or a DAS score of 4. The Potency of a modified BoNT/A may be also expressed as the EC₅₀ dose in a cellular assay measuring SNARE cleavage by the neurotoxin, for example the EC₅₀ dose in a cellular assay measuring SNAP25 cleavage by a modified BoNT/A.

The duration of action of a modified BoNT/A may be expressed as the time required for retrieving a DAS score of 0 after administration of a given dose of neurotoxin, for example the minimal dose of neurotoxin leading to a DAS score of 4, to a mouse gastrocnemius/soleus complex.

Thus, in one embodiment, a modified BoNT/A of the present invention has a Safety Ratio that is greater than 7 (for example, at least 8, 9, 10, 15, 20, 25, 30, 35, 40, 45 or 50), wherein Safety Ratio is calculated as: dose of toxin required for −10% bodyweight change (pg/mouse) divided by DAS ED₅₀ (pg/mouse) [ED₅₀=dose required to produce a DAS score of 2]. For example, a modified BoNT/A may have a Safety Ratio of at least 8, 9, 10, 15, 20, 25, 30, 35, 40, 45 or 50.

In one embodiment, a modified BoNT/A of the present invention has a Safety Ratio of at least 10. In one embodiment, a modified BoNT/A of the present invention has a Safety Ratio of at least 15.

Preferably, the modified BoNT/A has a Safety Ratio of at least 10 (e.g. a Safety Ratio of 10), more preferably at least 12 or 13 (e.g. 14-15).

The modified BoNT/A may have a Safety Ratio of greater than 7 up to 50 e.g. 8-45, 10-20 or 12-15.

In use, the modified BoNT/A of the invention is in a di-chain form.

A modified BoNT/A for use in the invention may comprise a polypeptide sequence having at least 70% sequence identity to a polypeptide sequence selected from SEQ ID NOs: 3-7. For example, a polypeptide sequence having at least 80%, 90%, 95% or 99.9% sequence identity to a polypeptide sequence selected from SEQ ID NOs: 3-7. Preferably, a modified BoNT/A for use in the invention may comprise (more preferably consist of) a polypeptide sequence selected from SEQ ID NOs: 3-7. Of said modified BoNT/As, SEQ ID NO: 6 is preferred.

Thus, it is preferred that the modified BoNT/A comprises a polypeptide sequence having at least 70% sequence identity to SEQ ID NO: 6. More preferably, a polypeptide sequence having at least 80%, 90%, 95% or 99.9% sequence identity to SEQ ID NO: 6. Most preferably, a modified BoNT/A for use in the invention may comprise (more preferably consist of) SEQ ID NO: 6.

A di-chain modified BoNT/A of the invention may comprise an L-chain portion of a polypeptide sequence having at least 70%, 80%, 90%, 95%, 99.9%, or 100% sequence identity to any one of SEQ ID NOs: 3-7 constituting a first chain of the di-chain modified BoNT/A, and may comprise the H_{N} and H_{C} domains of a polypeptide sequence having at least 70%, 80%, 90%, 95%, 99.9%, or 100% sequence identity to any one of SEQ ID NOs: 3-7 together constituting a second chain of the di-chain modified BoNT/A, wherein the first and second chains are joined together by a di-sulphide bond.

Where cleavage occurs at more than one position (preferably at two positions) within the activation loop of a modified BoNT/A comprising a polypeptide sequence having at least 70%, 80%, 90%, 95%, 99.9%, or 100% sequence identity to any one of SEQ ID NOs: 3-7, a small fragment of the C-terminal L-chain portion of the sequence having at least 70%, 80%, 90%, 95%, 99.9%, or 100% sequence identity to any one of SEQ ID NOs: 3-7 may be absent from the di-chain modified BoNT/A. In view of this, the sequence of the di-chain modified BoNT/A (e.g. comprising a polypeptide sequence having at least 70%, 80%, 90%, 95%, 99.9%, or 100% sequence identity to any one of SEQ ID NOs: 3-7) may be slightly different to that of the corresponding single-chain modified BoNT/A comprising a polypeptide sequence having at least 70%, 80%, 90%, 95%, 99.9%, or 100% sequence identity to any one of SEQ ID NOs: 3-7. The small fragment may be 1-15 amino acids. In particular, in one embodiment, when Lys-C is used to covert a single-chain modified BoNT/A into a di-chain clostridial neurotoxin, the small fragment of the C-terminal L-chain portion of the sequence that is absent may be SEQ ID NO: 9 or 10.

Preferably, a di-chain modified BoNT/A of the invention may comprise an L-chain portion of a polypeptide sequence having at least 70%, 80%, 90%, 95%, 99.9%, or 100% sequence identity to SEQ ID NO: 6 constituting a first chain of the di-chain modified BoNT/A, and may comprise the H_{N} and H_{C} domains of a polypeptide sequence having at least 70%, 80%, 90%, 95%, 99.9%, or 100% sequence identity to SEQ ID NO: 6 together constituting a second chain of the di-chain modified BoNT/A, wherein the first and second chains are joined together by a di-sulphide bond.

Where cleavage occurs at more than one position (preferably at two positions) within the activation loop of a modified BoNT/A comprising a polypeptide sequence having at least 70%, 80%, 90%, 95%, 99.9%, or 100% sequence identity to SEQ ID NO: 6, a small fragment of the C-terminal L-chain portion of the sequence having at least 70%, 80%, 90%, 95%, 99.9%, or 100% sequence identity to SEQ ID NO: 6 may be absent from the di-chain modified BoNT/A. In view of this, the sequence of the di-chain modified BoNT/A (e.g. comprising a polypeptide sequence having at least 70%, 80%, 90%, 95%, 99.9%, or 100% sequence identity to SEQ ID NO: 6) may be slightly different to that of the corresponding single-chain modified BoNT/A comprising a polypeptide sequence having at least 70%, 80%, 90%, 95%, 99.9%, or 100% sequence identity to SEQ ID NO: 6. The small fragment may be 1-15 amino acids. In particular, in one embodiment, when Lys-C is used to covert a single-chain modified BoNT/A into a di-chain modified BoNT/A, the small fragment of the C-terminal L-chain portion of the sequence that is absent may be SEQ ID NO: 9 or 10.

In a particularly preferred embodiment, a di-chain modified BoNT/A comprises (or consists of) a light-chain comprising a polypeptide sequence having at least 70%, 80%, 90%, 95%, or 99.9% sequence identity to SEQ ID NO: 11 or 12 (preferably SEQ ID NO: 11) and a heavy-chain comprising a polypeptide sequence having at least 70%, 80%, 90%, 95%, or 99.9% sequence identity to SEQ ID NO: 13, wherein the light-chain and heavy-chain are joined together by a di-sulphide bond. More preferably, a di-chain modified BoNT/A comprises (or consists of) a light-chain comprising SEQ ID NO: 11 or 12 (preferably SEQ ID NO: 11) and a heavy-chain comprising SEQ ID NO: 13, wherein the light-chain and heavy-chain are joined together by a di-sulphide bond. Even more preferably, a di-chain modified BoNT/A comprises (or consists of) a light-chain having SEQ ID NO: 11 and a heavy-chain having SEQ ID NO: 13, wherein the light-chain and heavy-chain are joined together by a di-sulphide bond. The di-sulphide bond is preferably formed by and/or is between cysteine residue 429 of SEQ ID NO: 11 or 12 and cysteine residue 6 of SEQ ID NO: 13.

In a preferred embodiment, a modified BoNT/A of the invention does not comprise a therapeutic or diagnostic agent (e.g. a nucleic acid, protein, peptide or small molecule therapeutic or diagnostic agent) additional to the light-chain and heavy-chain. For example, in one embodiment, the modified BoNT/A may not comprise a covalently or non-covalently associated therapeutic or diagnostic agent. Thus, a modified BoNT/A of the invention preferably does not function as a delivery vehicle for a further therapeutic or diagnostic agent.

In embodiments where a modified BoNT/A described herein has a tag for purification (e.g. a His-tag) and/or a linker, said tag and/or linker are optional.

The modified BoNT/A may be free from complexing proteins that are present in a naturally occurring clostridial neurotoxin complex. Examples of such complexing proteins include a neurotoxin-associated proteins (NAP) and a nontoxic-nonhemagglutinin component (NTNH). However, it is preferred that the modified BoNT/A is a recombinant modified BoNT/A.

The modified BoNT/A of the present invention can be produced using recombinant nucleic acid technologies. Thus, in one embodiment, a modified BoNT/A (as described herein) is a recombinant modified BoNT/A.

In one embodiment a nucleic acid (for example, DNA) comprising a nucleic acid sequence encoding a modified BoNT/A is provided. In one embodiment, the nucleic acid sequence is prepared as part of a DNA vector comprising a promoter and a terminator. The nucleic acid sequence may be selected from any of the nucleic acid sequences described herein.

In a preferred embodiment, the vector has a promoter selected from:

| Promoter | Induction Agent | Typical Induction Condition | |
|---|---|---|---|
| Tac (hybrid) | IPTG | 0.2 mM | (0.05-2.0 mM) |
| AraBAD | L-arabinose | 0.2% | (0.002-0.4%) |
| T7-lac operator | IPTG | 0.2 mM | (0.05-2.0 mM) |

In another preferred embodiment, the vector has a promoter selected from:

| Promoter | Induction Agent | Typical Induction Condition | |
|---|---|---|---|
| Tac (hybrid) | IPTG | 0.2 mM | (0.05-2.0 mM) |
| AraBAD | L-arabinose | 0.2% | (0.002-0.4%) |
| T7-lac operator | IPTG | 0.2 mM | (0.05-2.0 mM) |
| T5-lac operator | IPTG | 0.2 mM | (0.05-2.0 mM) |

The nucleic acid molecules may be made using any suitable process known in the art. Thus, the nucleic acid molecules may be made using chemical synthesis techniques. Alternatively, the nucleic acid molecules of the invention may be made using molecular biology techniques.

The DNA construct of the present invention is preferably designed in silico, and then synthesised by conventional DNA synthesis techniques.

The above-mentioned nucleic acid sequence information is optionally modified for codon-biasing according to the ultimate host cell (e.g. E. coli) expression system that is to be employed.

The terms “nucleotide sequence” and “nucleic acid” are used synonymously herein. Preferably the nucleotide sequence is a DNA sequence.

A modified BoNT/A of the invention may be present as a single-chain or as a di-chain. However, it is preferred that the modified BoNT/A is present as a di-chain in which the L-chain is linked to the H-chain (or component thereof, e.g. the H_{N} domain) via a di-sulphide bond.

Production of a single-chain modified BoNT/A having a light-chain and a heavy-chain may be achieved using a method comprising expressing a nucleic acid encoding a modified BoNT/A in an expression host, lysing the host cell to provide a host cell homogenate containing the single-chain modified BoNT/A, and isolating the single-chain modified BoNT/A. The single-chain modified BoNT/A may be proteolytically processed using a method comprising contacting a single-chain modified BoNT/A protein with a protease (e.g. Lys-C) that hydrolyses a peptide bond in the activation loop of the modified BoNT/A, thereby converting the single-chain modified BoNT/A into a corresponding di-chain modified BoNT/A (e.g. wherein the light chain and heavy chain are joined together by a disulphide bond). A di-chain modified BoNT/A is preferably obtainable by such a method.

Thus, a modified BoNT/A used in the invention is preferably a di-chain modified BoNT/A that has been produced from a single-chain BoNT/A, wherein the single-chain BoNT/A comprises or consists of a polypeptide sequence described herein. For example, it is preferred that the modified BoNT/A used in the invention is a di-chain modified BoNT/A that has been produced from a polypeptide comprising a polypeptide sequence having at least 70% (e.g. at least 80%, 90%, 95% or 99.9%) sequence identity to SEQ ID NO: 6. Most preferably, the modified BoNT/A used in the invention is a di-chain modified BoNT/A that has been produced from a polypeptide comprising (even more preferably consisting of) SEQ ID NO: 6. Accordingly, in some embodiments, the modified BoNT/A is a di-chain modified BoNT/A in which the light-chain (L-chain) is linked to the heavy-chain (H-chain) via a di-sulphide bond obtainable by a method comprising contacting a single-chain modified BoNT/A with a protease that hydrolyses a peptide bond in the activation loop thereof, thereby converting the single-chain modified BoNT/A into the corresponding di-chain modified BoNT/A. Accordingly, in some embodiments, the modified BoNT/A is a di-chain modified BoNT/A in which the light-chain (L-chain) is linked to the heavy-chain (H-chain) via a di-sulphide bond obtainable by a method comprising contacting a single-chain modified BoNT/A comprising SEQ ID NO: 6 with a protease that hydrolyses a peptide bond in the activation loop thereof, thereby converting the single-chain modified BoNT/A into the corresponding di-chain modified BoNT/A. In some embodiments, the modified BoNT/A is a di-chain modified BoNT/A in which the L-chain is linked to the H-chain via a di-sulphide bond obtainable by a method comprising contacting a single-chain modified BoNT/A consisting of SEQ ID NO: 6 with a protease that hydrolyses a peptide bond in the activation loop thereof, thereby converting the single-chain modified BoNT/A into the corresponding di-chain modified BoNT/A.

The protease used to cleave the activation loop is preferably Lys-C. Suitable proteases and methods for cleaving activation loops to produce di-chain clostridial neurotoxins are taught in WO 2014/080206, WO2014/079495, and EP2677029A2, which are incorporated herein by reference. Lys-C may cleave an activation loop C-terminal to one or more of the lysine residues present therein. Where Lys-C cleaves the activation loop more than once, the skilled person will appreciate that a small peptide of the activation loop of a di-chain modified BoNT/A may be absent when compared to a SEQ ID NO shown herein.

The term “obtainable” as used herein also encompasses the term “obtained”. In one embodiment the term “obtainable” means obtained.

The term “one or more” as used herein may mean at least 2, 3, 4, 5, 6, 7, 8, 9, 10, 15, or 20. In one embodiment, wherein “one or more” precedes a list, “one or more” may mean all of the members of the list. Similarly, the term “at least one” as used herein may mean at least 2, 3, 4, 5, 6, 7, 8, 9, 10, 15, or 20. In one embodiment, wherein “at least one” precedes a list, “at least one” may mean all of the members of the list.

The term “disorder” as used herein also encompasses a “disease”. In one embodiment the disorder is a disease.

The modified BoNT/A of the invention may be formulated in any suitable manner for administration to a subject, for example as part of a pharmaceutical composition. Such a pharmaceutical composition may comprise a modified BoNT/A of the invention and a pharmaceutically acceptable carrier, excipient, adjuvant, propellant and/or salt.

Compositions suitable for injection may be in the form of solutions, suspensions or emulsions, or dry powders which are dissolved or suspended in a suitable vehicle prior to use.

Fluid dosage forms are typically prepared utilising the modified BoNT/A and a pyrogen-free sterile vehicle. The modified BoNT/A, depending on the vehicle and concentration used, can be either dissolved or suspended in the vehicle. In preparing solutions the modified BoNT/A can be dissolved in the vehicle, the solution being made isotonic if necessary by addition of sodium chloride and sterilised by filtration through a sterile filter using aseptic techniques before filling into suitable sterile vials or ampoules and sealing. Alternatively, if solution stability is adequate, the solution in its sealed containers may be sterilised by autoclaving. Advantageously additives such as buffering, solubilising, stabilising, preservative or bactericidal, suspending or emulsifying agents and or local anaesthetic agents may be dissolved in the vehicle.

Dry powders, which are dissolved or suspended in a suitable vehicle prior to use, may be prepared by filling pre-sterilised ingredients into a sterile container using aseptic technique in a sterile area. Alternatively the ingredients may be dissolved into suitable containers using aseptic technique in a sterile area. The product is then freeze dried and the containers are sealed aseptically.

Parenteral suspensions, suitable for an administration route described herein, are prepared in substantially the same manner, except that the sterile components are suspended in the sterile vehicle, instead of being dissolved and sterilisation cannot be accomplished by filtration. The components may be isolated in a sterile state or alternatively it may be sterilised after isolation, e.g. by gamma irradiation.

Advantageously, a suspending agent for example polyvinylpyrrolidone may be included in the composition(s) to facilitate uniform distribution of the components.

In one aspect, the invention provides a unit dosage form of modified botulinum neurotoxin A (BoNT/A) (e.g. for treating adult limb spasticity), the unit dosage form comprising:
a. greater than 707 Units of modified BoNT/A, wherein 1 Unit is an amount of the modified BoNT/A that corresponds to the calculated median lethal dose (LD50) in mice; or
b. greater than 17,000 pg of modified BoNT/A; and
c. optionally a pharmaceutically acceptable carrier, excipient, adjuvant, and/or salt,
   wherein the modified BoNT/A comprises a BoNT/A light-chain and translocation domain, and a BoNT/B receptor binding domain (H_{C} domain).

It is preferred that the modified BoNT/A of the unit dosage form comprises a polypeptide sequence having at least 70% sequence identity to SEQ ID NO: 6. For example, a polypeptide sequence having at least 80%, 90%, 95% or 99.9% sequence identity to SEQ ID NO: 6. Most preferably, a modified BoNT/A may comprise (more preferably consist of) SEQ ID NO: 6.

A unit dosage form may comprise greater than 707 Units of modified BoNT/A. An upper limit of said range may be 1664, 1650, 1600, 1550, 1500, 1450, 1400, 1350, 1300, 1250, 1150, 1100, 1050, 1000, 950, 900, 850, 800 or 750 Units of modified BoNT/A, preferably the upper limit is 1500 Units. A lower limit of said range may be 725, 750, 775, 800, 825, 850, 875, 900, 925, 950, 975, 1000, 1025, 1050, 1075, 1100, 1250, 1300, 1350, 1400, 1450, 1500, 1550, 1600 or 1650 Units of modified BoNT/A, preferably the lower limit is 725 Units, 949 Units, or 1,381 Units. Preferably, a unit dosage form comprises greater than 707 Units up to 1664 Units of modified BoNT/A, for example greater than 707 Units up to 1,498 Units or 832 Units to 1622 Units. A unit dosage form may comprise 915 to 1581 Units, preferably 949 to 1581 Units or 1414 to 1581 Units of modified BoNT/A. A unit dosage form may comprise 998 to 1,498 Units or 1,040 to 1,498 Units of modified BoNT/A. In preferred embodiments, a unit dosage form comprises 998, 1,040, 1,248 or 1,498 Units of modified BoNT/A. In more preferred embodiments, a unit dosage form comprises 1,248 or 1,498 Units (e.g. 1,498 Units) of modified BoNT/A.

It is more preferred that a unit dosage form comprises 1,248 Units to 1,664 Units, such as 1,456 Units to 1,539 Units of modified BoNT/A. More preferably, the unit dosage form comprises 1,498 Units of modified BoNT/A.

It is even more preferred that a unit dosage form comprises 832 Units to 1,248 Units, such as 998 Units to 1,082 Units of modified BoNT/A. Most preferably, the unit dosage form comprises 1,040 Units of modified BoNT/A.

A unit dosage form may comprise greater than 17,000 pg of modified BoNT/A. An upper limit of said range may be 40,000, 39,000, 38,000, 37,000, 36,000, 35,000, 30,000, 25,000, 24,000, 22,000, 20,000, or 18,000, pg of modified BoNT/A, preferably the upper limit is 38,000 pg. A lower limit of said range may be 17,500, 18,000, 20,000, 22,000, 24,000, 25,000, 26,000, 27,000, 28,000, 29,000, 30,000, 35,000, 36,000, 37,000, 38,000 or 39,000 pg of modified BoNT/A, preferably the lower limit is 17,500 pg, 22,753 pg, 22,790 pg or 33,1818 pg. Preferably, the unit dosage form comprises greater than 17,000 pg up to 40,000 pg, e.g. greater than 17,000 pg up to 36,000 pg, or 20,000 pg to 39,000 pg, of modified BoNT/A. A unit dosage form may comprise 22,000 to 38,000 pg, preferably 23,000 to 38,000 pg, or 34,000 to 38,000 pg of modified BoNT/A. A unit dosage form may comprise 24,000 to 36,000 pg or 25,000 to 36,000 pg of modified BoNT/A. In preferred embodiments, a unit dosage form comprises 24,000, 25,000, 30,000 or 36,000 pg of modified BoNT/A. In more preferred embodiments, a unit dosage form comprises 30,000 or 36,000 pg (e.g. 36,000 pg) of modified BoNT/A.

It is more preferred that a unit dosage form comprises 30,000 pg to 40,000 pg, such as 35,000 pg to 37,000 pg of modified BoNT/A. More preferably, the unit dose dosage form comprises 36,000 pg of modified BoNT/A.

It is even more preferred that a unit dose form comprises 20,000 pg to 30,000 pg, such as 24,000 pg to 26,000 pg of modified BoNT/A. Most preferably, the unit dose form comprises 25,000 pg of modified BoNT/A.

The above unit dosage forms may be suitable for treating an adult subject.

The invention provides, in one aspect, the above-mentioned unit dosage form for use in treating adult limb spasticity. Also provided are corresponding methods for treating adult limb spasticity, and uses thereof in the manufacture of a medicament for treating adult limb spasticity. The treatment is preferably in accordance with a treatment described herein.

As discussed earlier, the unit dose for paediatric applications may be 50% or less than that indicated above. In some embodiments, the unit dose is typically 70% or less (e.g. 67% or less) than that used when treating adult limb spasticity.

In another aspect, the invention provides a unit dosage form of modified botulinum neurotoxin A (BoNT/A) for treating a paediatric subject (e.g. for treating paediatric limb spasticity), the unit dosage form comprising:
a. greater than 353.5 Units of modified BoNT/A, wherein 1 Unit is an amount of the modified BoNT/A that corresponds to the calculated median lethal dose (LD₅₀) in mice; or
b. greater than 8,500 pg of modified BoNT/A; and
c. optionally a pharmaceutically acceptable carrier, excipient, adjuvant, and/or salt,
   wherein the modified BoNT/A comprises a BoNT/A light-chain and translocation domain, and a BoNT/B receptor binding domain (H_{C} domain).

It is preferred that the modified BoNT/A of the unit dosage form comprises a polypeptide sequence having at least 70% sequence identity to SEQ ID NO: 6. For example, a polypeptide sequence having at least 80%, 90%, 95% or 99.9% sequence identity to SEQ ID NO: 6. Most preferably, a modified BoNT/A may comprise (more preferably consist of) SEQ ID NO: 6.

A unit dosage form for treating a paediatric subject may comprise greater than 353.5 Units of modified BoNT/A. An upper limit said range may be 832, 825, 800, 775, 750, 725, 700, 675, 650, 625, 600, 575, 550, 525, 500, 475, 450, or 425 Units of modified BoNT/A, preferably the upper limit is 749, 750 or 790 Units. A lower limit of said range may be 364, 375, 400, 425, 450, 475, 500, 525, 550, 575, 600, 625, 650, 675, 700, 725, 750, 775, 800, 825 or 830 Units of modified BoNT/A, preferably the lower limit is 364 Units, 416 Units, 474.5 Units, 475 Units, 690 Units, or 690.5 Units. Preferably, a unit dosage form comprises 353.5 Units to 832 Units of modified BoNT/A, for example greater than 353.5 Units up to 749 Units or 416 Units to 811 Units. A unit dose of modified BoNT/A for treating paediatric limb spasticity may comprise 457.5 Units to 749 Units, 458 Units to 790 Units, or 458 Units to 791 Units, preferably 474.5 Units to 749 Units, 707 Units to 749 Units, 475 Units to 790 Units, 475 Units to 791 Units, 707 Units to 790 Units, or 707 Units to 791 Units. In preferred embodiments, a unit dosage form comprises 499, 520, 624 or 749 Units of modified BoNT/A. In more preferred embodiments, a unit dosage form comprises 624 or 749 Units (e.g. 749 Units) of modified BoNT/A.

It is more preferred that a unit dosage form for treating paediatric limb spasticity comprises 624 Units to 832 Units, such as 728 Units to 770 Units of modified BoNT/A. More preferably, the unit dosage form for treating paediatric limb spasticity comprises 749 Units of modified BoNT/A.

It is even more preferred that a unit dosage form for treating paediatric limb spasticity comprises 416 Units to 624 Units, such as 499 Units to 541 Units of modified BoNT/A. Most preferably, the unit dosage form for treating paediatric limb spasticity comprises 520 Units of modified BoNT/A.

A unit dosage form for treating a paediatric subject may comprise greater than 8,500 pg of modified BoNT/A. An upper limit of said range may be 20,000, 19,500, 19,000, 18,500, 18,000, 17,500, 15,000, 12,500, 12,000, 11,000, 10,000 or 9,000 pg of modified BoNT/A, preferably the upper limit is 19,000 pg. A lower limit of said range may be 8,750, 9,000, 10,000, 11,000, 12,000, 12,500, 13,000, 13,500, 14,000, 14,500, 15,000, 17,500, 18,000, 18,500, 19,000 or 19,500 pg of modified BoNT/A, preferably the lower limit is 8,750 pg, 11,376 pg, 11,377 pg, 11,395 pg, 11,400 pg, 16,590 pg or 16,591 pg. Preferably, the unit dosage form comprises greater than 8,500 pg up to 20,000 pg, e.g. greater than 8,500 pg up to 18,000 pg, or 10,000 to 19,500 of modified BoNT/A. A unit dosage form may comprise 11,000 to 19,000 pg, preferably 11,500 pg to 19,000 pg, or 17,000 to 19,000 pg of modified BoNT/A. A unit dosage form may comprise 12,000 pg to 18,000 pg or 12,500 pg to 18,000 pg of modified BoNT/A. The unit dosage form may comprise 12,500 pg to 20,000 pg of modified BoNT/A. In preferred embodiments, a unit dosage form comprises 12,000, 12,500, 15,000 or 18,000 pg of modified BoNT/A. In more preferred embodiments, a unit dosage form comprises 15,000 or 18,000 pg (e.g. 18,000 pg) of modified BoNT/A.

It is more preferred that a unit dosage form for treating paediatric limb spasticity comprises 15,000 pg to 20,000 pg, such as 17,500 to 18,500 pg of modified BoNT/A. More preferably, the unit dosage form for treating paediatric limb spasticity comprises 18,000 pg of modified BoNT/A.

It is even more preferred that a unit dosage form for treating paediatric limb spasticity comprises 10,000 pg to 15,000 pg, such as 12,000 pg to 13,000 pg of modified BoNT/A. More preferably, a unit dosage form for treating paediatric limb spasticity comprises 12,500 pg of modified BoNT/A.

The invention provides, in one aspect, the above-mentioned unit dosage form for use in treating paediatric limb spasticity. Also provided are corresponding methods for treating paediatric limb spasticity, and uses thereof in the manufacture of a medicament for treating paediatric limb spasticity. The treatment is preferably in accordance with a treatment described herein.

A modified BoNT/A present in the unit dosage form is preferably in a di-chain form. Thus, said modified BoNT/A is preferably a di-chain modified BoNT/A described herein (and/or produced as described herein).

The unit dosage form is preferably provided as a dry powder.

In another aspect, the invention provides a kit comprising:
a. the unit dosage form according to the present invention; and
b. instructions for use of the same in treating limb spasticity; and
c. optionally a diluent.

In one aspect, the invention provides a modified botulinum neurotoxin A (BoNT/A) for use in treating adult limb spasticity, wherein the modified BoNT/A is administered by intramuscular injection to a plurality of affected muscles of a subject,
wherein the modified BoNT/A is administered by way of a unit dose of 10,000 pg to 20,000 pg (e.g. 14,000 pg to 16,000 pg, preferably 15,000 pg) of modified BoNT/A at the plurality of affected muscles,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius,
preferably wherein at least one of the plurality of affected muscles is selected from: the adductor magnus, a hamstring muscle (e.g. the biceps femoris, the semimembranosus, and/or the semitendinosus), and the rectus femoris; and
wherein a single unit dose is administered at an affected first group muscle and/or multiple unit doses are administered at an affected second group muscle, and wherein the total dose administered during the treatment is up to 300,000 pg (e.g. up to 240,000 pg, preferably up to 225,000 pg), and
wherein the modified BoNT/A comprises a BoNT/A light-chain and translocation domain, and a BoNT/B receptor binding domain (H_{C} domain).

In one aspect, the invention provides a method for treating adult limb spasticity, the method comprising administering a modified botulinum neurotoxin A (BoNT/A) by intramuscular injection to a plurality of affected muscles of a subject,
wherein the modified BoNT/A is administered by way of a unit dose of 10,000 pg to 20,000 pg (e.g. 14,000 pg to 16,000 pg, preferably 15,000 pg) of modified BoNT/A at the plurality of affected muscles,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius,
preferably wherein at least one of the plurality of affected muscles is selected from: the adductor magnus, a hamstring muscle (e.g. the biceps femoris, the semimembranosus, and/or the semitendinosus), and the rectus femoris; and
wherein a single unit dose is administered at an affected first group muscle and/or multiple unit doses are administered at an affected second group muscle, and wherein the total dose administered during the treatment is up to 300,000 pg (e.g. up to 240,000 pg, preferably up to 225,000 pg), and
wherein the modified BoNT/A comprises a BoNT/A light-chain and translocation domain, and a BoNT/B receptor binding domain (H_{C} domain).

In one aspect, the invention provides a use of a modified botulinum neurotoxin A (BoNT/A) in the manufacture of a medicament for treating adult limb spasticity, wherein the modified BoNT/A is administered by intramuscular injection to a plurality of affected muscles of a subject,
wherein the modified BoNT/A is administered by way of a unit dose of 10,000 pg to 20,000 pg (e.g. 14,000 pg to 16,000 pg, preferably 15,000 pg) of modified BoNT/A at the plurality of affected muscles,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius,
preferably wherein at least one of the plurality of affected muscles is selected from: the adductor magnus, a hamstring muscle (e.g. the biceps femoris, the semimembranosus, and/or the semitendinosus), and the rectus femoris; and
wherein a single unit dose is administered at an affected first group muscle and/or multiple unit doses are administered at an affected second group muscle, and wherein the total dose administered during the treatment is up to 300,000 pg (e.g. up to 240,000 pg, preferably up to 225,000 pg), and
wherein the modified BoNT/A comprises a BoNT/A light-chain and translocation domain, and a BoNT/B receptor binding domain (H_{C} domain).

In said foregoing aspects, preferably the unit dose is 15,000 pg and the total dose is up to 225,000 pg (e.g. the total dose may be 225,000 pg).

In one aspect, the invention provides a modified botulinum neurotoxin A (BoNT/A) for use in treating adult limb spasticity, wherein the modified BoNT/A is administered by intramuscular injection to a plurality of affected muscles of a subject,
wherein the modified BoNT/A is administered by way of a unit dose of 416 Units to 832 Units (e.g. 582 Units to 666 Units, preferably 624 Units) of modified BoNT/A at the plurality of affected muscles, and wherein 1 Unit is an amount of the modified BoNT/A that corresponds to the calculated median lethal dose (LD₅₀) in mice,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius,
preferably wherein at least one of the plurality of affected muscles is selected from: the adductor magnus, a hamstring muscle (e.g. the biceps femoris, the semimembranosus, and/or the semitendinosus), and the rectus femoris; and
wherein a single unit dose is administered at an affected first group muscle and/or multiple unit doses are administered at an affected second group muscle, and wherein the total dose administered during the treatment is up to 12,480 Units (e.g. up to 9,990 Units, preferably up to 9,360 Units), and
wherein the modified BoNT/A comprises a BoNT/A light-chain and translocation domain, and a BoNT/B receptor binding domain (H_{C} domain).

In one aspect, the invention provides a method for treating adult limb spasticity, the method comprising administering a modified botulinum neurotoxin A (BoNT/A) by intramuscular injection to a plurality of affected muscles of a subject,
wherein the modified BoNT/A is administered by way of a unit dose of 416 Units to 832 Units (e.g. 582 Units to 666 Units, preferably 624 Units) of modified BoNT/A at the plurality of affected muscles, and wherein 1 Unit is an amount of the modified BoNT/A that corresponds to the calculated median lethal dose (LD₅₀) in mice,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius,
preferably wherein at least one of the plurality of affected muscles is selected from: the adductor magnus, a hamstring muscle (e.g. the biceps femoris, the semimembranosus, and/or the semitendinosus), and the rectus femoris; and
wherein a single unit dose is administered at an affected first group muscle and/or multiple unit doses are administered at an affected second group muscle, and wherein the total dose administered during the treatment is up to 12,480 Units (e.g. up to 9,990 Units, preferably up to 9,360 Units), and
wherein the modified BoNT/A comprises a BoNT/A light-chain and translocation domain, and a BoNT/B receptor binding domain (H_{C} domain).

In one aspect, the invention provides a use of a modified botulinum neurotoxin A (BoNT/A) in the manufacture of a medicament for treating adult limb spasticity, wherein the modified BoNT/A is administered by intramuscular injection to a plurality of affected muscles of a subject,
wherein the modified BoNT/A is administered by way of a unit dose of 416 Units to 832 Units (e.g. 582 Units to 666 Units, preferably 624 Units) of modified BoNT/A at the plurality of affected muscles, and wherein 1 Unit is an amount of the modified BoNT/A that corresponds to the calculated median lethal dose (LD₅₀) in mice,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius,
preferably wherein at least one of the plurality of affected muscles is selected from: the adductor magnus, a hamstring muscle (e.g. the biceps femoris, the semimembranosus, and/or the semitendinosus), and the rectus femoris; and
wherein a single unit dose is administered at an affected first group muscle and/or multiple unit doses are administered at an affected second group muscle, and wherein the total dose administered during the treatment is up to 12,480 Units (e.g. up to 9,990 Units, preferably up to 9,360 Units), and
wherein the modified BoNT/A comprises a BoNT/A light-chain and translocation domain, and a BoNT/B receptor binding domain (H_{C} domain).

In said foregoing aspects, preferably the unit dose is 624 Units and the total dose is up to 9,360 Units (e.g. the total dose may be 9,360 Units).

In one aspect, the invention provides a modified botulinum neurotoxin A (BoNT/A) for use in treating paediatric limb spasticity, wherein the modified BoNT/A is administered by intramuscular injection to a plurality of affected muscles of a subject,
wherein the modified BoNT/A is administered by way of a unit dose of 5,000 pg to 10,000 pg (e.g. 7,000 pg to 8,000 pg, preferably 7,500 pg) of modified BoNT/A at the plurality of affected muscles,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius,
preferably wherein at least one of the plurality of affected muscles is selected from: the adductor magnus, a hamstring muscle (e.g. the biceps femoris, the semimembranosus, and/or the semitendinosus), and the rectus femoris; and
wherein a single unit dose is administered at an affected first group muscle and/or multiple unit doses are administered at an affected second group muscle, and wherein the total dose administered during the treatment is up to 150,000 pg (e.g. up to 120,000 pg, preferably up to 112,500 pg), and
wherein the modified BoNT/A comprises a BoNT/A light-chain and translocation domain, and a BoNT/B receptor binding domain (H_{C} domain).

In one aspect, the invention provides a method for treating paediatric limb spasticity, the method comprising administering a modified botulinum neurotoxin A (BoNT/A) by intramuscular injection to a plurality of affected muscles of a subject,
wherein the modified BoNT/A is administered by way of a unit dose of 5,000 pg to 10,000 pg (e.g. 7,000 pg to 8,000 pg, preferably 7,500 pg) of modified BoNT/A at the plurality of affected muscles,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius,
preferably wherein at least one of the plurality of affected muscles is selected from: the adductor magnus, a hamstring muscle (e.g. the biceps femoris, the semimembranosus, and/or the semitendinosus), and the rectus femoris; and
wherein a single unit dose is administered at an affected first group muscle and/or multiple unit doses are administered at an affected second group muscle, and wherein the total dose administered during the treatment is up to 150,000 pg (e.g. up to 120,000 pg, preferably up to 112,500 pg), and
wherein the modified BoNT/A comprises a BoNT/A light-chain and translocation domain, and a BoNT/B receptor binding domain (H_{C} domain).

In one aspect, the invention provides a use of a modified botulinum neurotoxin A (BoNT/A) in the manufacture of a medicament for treating paediatric limb spasticity, wherein the modified BoNT/A is administered by intramuscular injection to a plurality of affected muscles of a subject,
wherein the modified BoNT/A is administered by way of a unit dose of 5,000 pg to 10,000 pg (e.g. 7,000 pg to 8,000 pg, preferably 7,500 pg) of modified BoNT/A at the plurality of affected muscles,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius,
preferably wherein at least one of the plurality of affected muscles is selected from: the adductor magnus, a hamstring muscle (e.g. the biceps femoris, the semimembranosus, and/or the semitendinosus), and the rectus femoris; and
wherein a single unit dose is administered at an affected first group muscle and/or multiple unit doses are administered at an affected second group muscle, and wherein the total dose administered during the treatment is up to 150,000 pg (e.g. up to 120,000 pg, preferably up to 112,500 pg), and
wherein the modified BoNT/A comprises a BoNT/A light-chain and translocation domain, and a BoNT/B receptor binding domain (H_{C} domain).

In said foregoing aspects, preferably the unit dose is 7,500 pg and the total dose is up to 112,500 pg (e.g. the total dose may be 112,500 pg).

In one aspect, the invention provides a modified botulinum neurotoxin A (BoNT/A) for use in treating paediatric limb spasticity, wherein the modified BoNT/A is administered by intramuscular injection to a plurality of affected muscles of a subject,
wherein the modified BoNT/A is administered by way of a unit dose of 208 Units to 416 Units (e.g. 291 Units to 333 Units, preferably 312 Units) of modified BoNT/A at the plurality of affected muscles, and wherein 1 Unit is an amount of the modified BoNT/A that corresponds to the calculated median lethal dose (LD₅₀) in mice,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius,
preferably wherein at least one of the plurality of affected muscles is selected from: the adductor magnus, a hamstring muscle (e.g. the biceps femoris, the semimembranosus, and/or the semitendinosus), and the rectus femoris; and
wherein a single unit dose is administered at an affected first group muscle and/or multiple unit doses are administered at an affected second group muscle, and wherein the total dose administered during the treatment is up to 6,240 Units (e.g. up to 4,995 Units, preferably up to 4,680 Units), and
wherein the modified BoNT/A comprises a BoNT/A light-chain and translocation domain, and a BoNT/B receptor binding domain (H_{C} domain).

In one aspect, the invention provides a method for treating paediatric limb spasticity, the method comprising administering a modified botulinum neurotoxin A (BoNT/A) by intramuscular injection to a plurality of affected muscles of a subject,
wherein the modified BoNT/A is administered by way of a unit dose of 208 Units to 416 Units (e.g. 291 Units to 333 Units, preferably 312 Units) of modified BoNT/A at the plurality of affected muscles, and wherein 1 Unit is an amount of the modified BoNT/A that corresponds to the calculated median lethal dose (LD₅₀) in mice,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius,
preferably wherein at least one of the plurality of affected muscles is selected from: the adductor magnus, a hamstring muscle (e.g. the biceps femoris, the semimembranosus, and/or the semitendinosus), and the rectus femoris; and
wherein a single unit dose is administered at an affected first group muscle and/or multiple unit doses are administered at an affected second group muscle, and wherein the total dose administered during the treatment is up to 6,240 Units (e.g. up to 4,995 Units, preferably up to 4,680 Units), and
wherein the modified BoNT/A comprises a BoNT/A light-chain and translocation domain, and a BoNT/B receptor binding domain (H_{C} domain).

In one aspect, the invention provides a use of a modified botulinum neurotoxin A (BoNT/A) in the manufacture of a medicament for treating paediatric limb spasticity, wherein the modified BoNT/A is administered by intramuscular injection to a plurality of affected muscles of a subject,
wherein the modified BoNT/A is administered by way of a unit dose of 208 Units to 416 Units (e.g. 291 Units to 333 Units, preferably 312 Units) of modified BoNT/A at the plurality of affected muscles, and wherein 1 Unit is an amount of the modified BoNT/A that corresponds to the calculated median lethal dose (LD₅₀) in mice,
wherein the plurality of affected muscles are selected from:
   a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
   a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius,
preferably wherein at least one of the plurality of affected muscles is selected from: the adductor magnus, a hamstring muscle (e.g. the biceps femoris, the semimembranosus, and/or the semitendinosus), and the rectus femoris; and
wherein a single unit dose is administered at an affected first group muscle and/or multiple unit doses are administered at an affected second group muscle, and wherein the total dose administered during the treatment is up to 6,240 Units (e.g. up to 4,995 Units, preferably up to 4,680 Units), and
wherein the modified BoNT/A comprises a BoNT/A light-chain and translocation domain, and a BoNT/B receptor binding domain (H_{C} domain).

In said foregoing aspects, preferably the unit dose is 312 Units and the total dose is up to 4,680 Units (e.g. the total dose may be 4,680 Units).

In any of the above aspects, limb spasticity may be treated for a longer duration than that treated by an unmodified BoNT/A (e.g. SEQ ID NO: 2 [such as a di-chain form of SEQ ID NO: 2]).

The total number of unit doses administered in a given treatment may be up to 15× the unit dose. In other words, in one embodiment, 15× single unit doses may be administered at 15× injections sites. In another embodiment, 15× single unit doses may be administered at more than 15× injections sites. The total number of unit doses will be divided according to the muscles treated, e.g. 2× unit doses may be administered to the latissimus dorsi, 2× to the subscapularis, 2× to the pectoralis major, and 1× to the flexor carpi ulnaris yielding a total multiple of unit doses administered of 7×. For example, the total number of unit doses administered may be up to 14×, 13×, 12×, 11×, 10×, 9×, 8× or 7×. The total number of unit doses administered may be at least 2×, 3×, 4×, 5×, 6×, 7× the unit dose, preferably at least 2×. The total number of unit doses administered may be 2× to 15×, 7× to 15× or 10× to 14×. Preferably, the number of unit doses administered is 15×.

Embodiments related to the various therapeutic uses of the invention are intended to be applied equally to the methods, compositions (e.g. unit dosage forms), and kits of the invention and vice versa.

### Sequence Homology

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22(22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264(4) J. Mol. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501-509 (1992); Gibbs sampling, see, e.g., C. E. Lawrence et al., Detecting Subtle Sequence Signals: A Gibbs Sampling Strategy for Multiple Alignment, 262(5131) Science 208-214 (1993); Align-M, see, e.g., Ivo Van Walle et al., Align-M—A New Algorithm for Multiple Alignment of Highly Divergent Sequences, 20(9) Bioinformatics:1428-1435 (2004).

Thus, percent sequence identity is determined by conventional methods. See, for example, Altschul et al., Bull. Math. Bio. 48: 603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown below (amino acids are indicated by the standard one-letter codes); preferably this method is used to align a sequence with a subject sequence herein (e.g. SEQ ID NO: 2) to define amino acid position numbering as described herein.

The “percent sequence identity” between two or more nucleic acid or amino acid sequences is a function of the number of identical positions shared by the sequences. Thus, % identity may be calculated as the number of identical nucleotides/amino acids divided by the total number of nucleotides/amino acids, multiplied by 100. Calculations of % sequence identity may also take into account the number of gaps, and the length of each gap that needs to be introduced to optimize alignment of two or more sequences. Sequence comparisons and the determination of percent identity between two or more sequences can be carried out using specific mathematical algorithms, such as BLAST, which will be familiar to a skilled person.

ALIGNMENT SCORES FOR DETERMINING SEQUENCE IDENTITY

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

The percent identity is then calculated as:

$$\frac{Total number of identical matches}{\text{?}} \times 100$$

$$\text{?} \text{indicates text missing or illegible when filed}$$

[length of the longer sequence plus the number of gaps introduced into the longer sequence in order to align the two sequences]

Substantially homologous polypeptides are characterized as having one or more amino acid substitutions, deletions or additions. These changes are preferably of a minor nature, that is conservative amino acid substitutions (see below) and other substitutions that do not significantly affect the folding or activity of the polypeptide; small deletions, typically of one to about 30 amino acids; and small amino- or carboxyl-terminal extensions, such as an amino-terminal methionine residue, a small linker peptide of up to about 20-25 residues, or an affinity tag.

CONSERVATIVE AMINO ACID SUBSTITUTIONS

| | |
|---|---|
| Basic: | arginine |
| | lysine |
| | histidine |
| Acidic: | glutamic acid |
| | aspartic acid |
| Polar: | glutamine |
| | asparagine |
| Hydrophobic: | leucine |
| | isoleucine |
| | valine |
| Aromatic: | phenylalanine |
| | tryptophan |
| | tyrosine |
| Small: | glycine |
| | alanine |
| | serine |
| | threonine |
| | methionine |

In addition to the 20 standard amino acids, non-standard amino acids (such as 4-hydroxyproline, 6-N-methyl lysine, 2-aminoisobutyric acid, isovaline and α-methyl serine) may be substituted for amino acid residues of the polypeptides of the present invention. A limited number of non-conservative amino acids, amino acids that are not encoded by the genetic code, and unnatural amino acids may be substituted for polypeptide amino acid residues. The polypeptides of the present invention can also comprise non-naturally occurring amino acid residues.

Non-naturally occurring amino acids include, without limitation, trans-3-methylproline, 2,4-methano-proline, cis-4-hydroxyproline, trans-4-hydroxy-proline, N-methylglycine, allo-threonine, methyl-threonine, hydroxy-ethylcysteine, hydroxyethylhomo-cysteine, nitro-glutamine, homoglutamine, pipecolic acid, tert-leucine, norvaline, 2-azaphenylalanine, 3-azaphenyl-alanine, 4-azaphenyl-alanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for amino acid residues of polypeptides of the present invention.

Essential amino acids in the polypeptides of the present invention can be identified according to procedures known in the art, such as site-directed mutagenesis or alanine-scanning mutagenesis (Cunningham and Wells, Science 244: 1081-5, 1989). Sites of biological interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related components (e.g. the translocation or protease components) of the polypeptides of the present invention.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

The headings provided herein are not limitations of the various aspects or embodiments of this disclosure.

Amino acids are referred to herein using the name of the amino acid, the three letter abbreviation or the single letter abbreviation. The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “enzyme”. The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Other definitions of terms may appear throughout the specification. Before the exemplary embodiments are described in more detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be defined only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise. Thus, for example, reference to “a botulinum neurotoxin A” includes a plurality of such candidate agents and reference to “the botulinum neurotoxin A” includes reference to one or more clostridial neurotoxins and equivalents thereof known to those skilled in the art, and so forth.

The publications discussed herein are provided solely for their disclosure prior to the filing date of the present application. Nothing herein is to be construed as an admission that such publications constitute prior art to the claims appended hereto.

### BRIEF DESCRIPTION OF THE DRAWINGS

Embodiments of the invention will now be described, by way of example only, with reference to the following Figures and Examples.

FIG. 1 shows the five most common upper limb spasticity clinical patterns involving upper arm joints.

FIG. 2 shows the FDA approved dosages of Dysport® for treating spasticity in adults.

FIG. 3 shows SDS-PAGE of purified recombinant BoNT/AB chimera 1, 2 and 3A (SEQ ID NO: 3, 4 and 5 respectively). Lanes are labelled “Marker” (molecular weight marker), “−DTT” (oxidised BoNT/AB chimera sample), and “+DTT” (reduced BoNT/AB chimera sample).

FIG. 4 shows cleavage of SNAP-25 in rat spinal cord neurones by recombinant BoNT/AB chimera 1, 2 and 3A (SEQ ID NO: 3, 4 and 5 converted into a di-chain form, respectively). Cultured rat primary spinal cord neurons (SCN) were exposed to various concentrations of recombinant BoNT/AB chimera 1, 2 or 3A for 24 hours, at 37° C. in a humidified atmosphere with 10% CO₂. Cells were then lysed with 1× NuPAGE buffer supplemented with DTT and Benzonase. The samples were transferred to microcentrifuge tubes, heated for 5 min at 90° C. on heat block and stored at −20° C., before analysis of SNAP-25 cleavage by Western blot. SNAP-25 was detected using a polyclonal antibody, that detects both the full length and cleaved forms of SNAP-25 (Sigma #S9684). Anti-rabbit HRP (Sigma #A6154) was used as the secondary antibody.

FIG. 5 shows mouse digit abduction scoring assay. Mice were injected into the gastrocnemius-soleus complex muscles of one hind limb, under short general anaesthesia; muscle weakening was measured on a 0-4 scale using the digit abduction score (DAS). DAS max values were determined for each dose and plotted against dose and the data were fitted to a 4-parameter logistic equation, ED50 and dose leading to DAS 4 (DAS 4 dose) values were determined.

FIG. 6 shows SDS-PAGE of purified recombinant BoNT/AB chimera 3B and 3C (SEQ ID NO: 6 and 7 respectively). Lanes are labelled “Marker” (molecular weight marker), “−DTT” (oxidised BoNT/AB chimera sample), and “+DTT” (reduced BoNT/AB chimera sample).

FIG. 7 shows cleavage of SNAP-25 by unmodified BoNT/A and BoNT/AB chimera 3B and 3C (SEQ ID NO: 2, 6 and 7 respectively) in human induced pluripotent stem cell derived peripheral neurons (PERI.4U—Axiogenesis, Germany). PERI.4U cells were exposed to various concentrations of recombinant BoNT/A, or BoNT/AB chimera 3B or 3C for 24 hours, at 37° C. in a humidified CO₂ atmosphere containing 5% CO₂. Cells were then lysed with 1× NuPAGE buffer supplemented with DTT and Benzonase. The samples were transferred to microcentrifuge tubes, heated for 5 min at 90° C. on heat block and stored at −20° C., before analysis of SNAP-25 cleavage by Western blot. SNAP-25 was detected using a polyclonal antibody, that detects both the full length and cleaved forms of SNAP-25 (Sigma #S9684). Anti-rabbit HRP (Sigma #A6154) was used as the secondary antibody.

FIG. 8 shows duration of muscle weakening over time in the mouse digit abduction scoring assay. Mice were injected into the gastrocnemius-soleus complex muscles of one hind limb, under short general anaesthesia; muscle weakening was measured on a 0-4 scale using the digit abduction score (DAS). Animals of the group injected with the lowest dose that induced during the first four days of injection a DAS of 4 were monitored until complete recovery of the muscle weakness to a DAS of 0 (no observed muscle weakness).

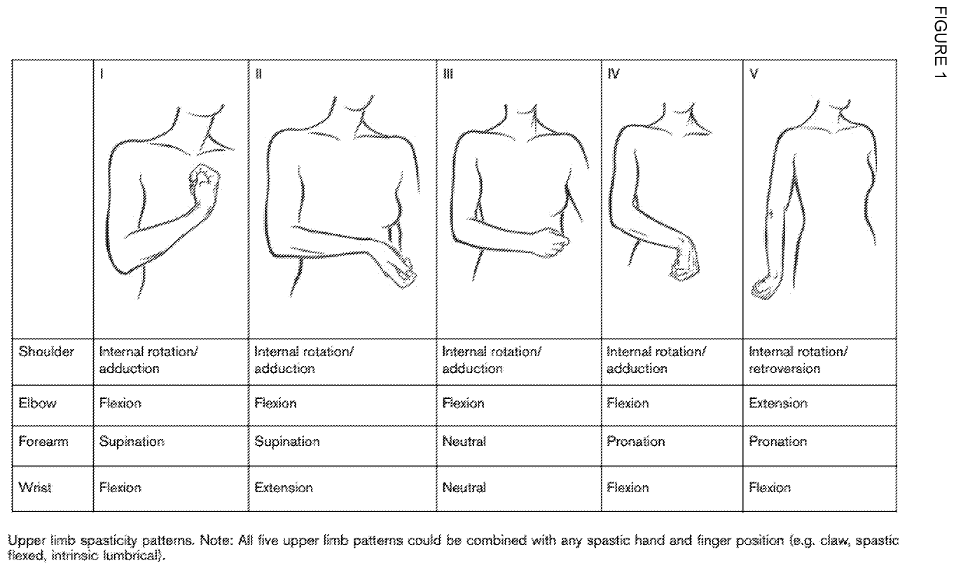

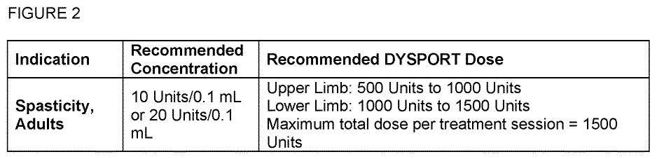

### SEQUENCE LISTING

Where an initial Met amino acid residue or a corresponding initial codon is indicated in any of the following SEQ ID NOs, said residue/codon is optional.

SEQ ID NO: 1
(Nucleotide Sequence of Unmodified BoNT/A)
ATGCCATTCGTCAACAAGCAATTCAACTACAAAGACCCAGTCAACGGCGTCGACATCGCATACATCAAGATTCCG
AACGCCGGTCAAATGCAGCCGGTTAAGGCTTTTAAGATCCACAACAAGATTTGGGTTATCCCGGAGCGTGACACC
TTCACGAACCCGGAAGAAGGCGATCTGAACCCGCCACCGGAAGCGAAGCAAGTCCCTGTCAGCTACTACGATTCG
ACGTACCTGAGCACGGATAACGAAAAAGATAACTACCTGAAAGGTGTGACCAAGCTGTTCGAACGTATCTACAGC
ACGGATCTGGGTCGCATGCTGCTGACTAGCATTGTTCGCGGTATCCCGTTCTGGGGTGGTAGCACGATTGACACC
GAACTGAAGGTTATCGACACTAACTGCATTAACGTTATTCAACCGGATGGTAGCTATCGTAGCGAAGAGCTGAAT
CTGGTCATCATTGGCCCGAGCGCAGACATTATCCAATTCGAGTGCAAGAGCTTTGGTCACGAGGTTCTGAATCTG
ACCCGCAATGGCTATGGTAGCACCCAGTACATTCGTTTTTCGCCGGATTTTACCTTCGGCTTTGAAGAGAGCCTG
GAGGTTGATACCAATCCGTTGCTGGGTGCGGGCAAATTCGCTACCGATCCGGCTGTCACGCTGGCCCATGAACTG
ATCCACGCAGGCCACCGCCTGTACGGCATTGCCATCAACCCAAACCGTGTGTTCAAGGTTAATACGAATGCATAC
TACGAGATGAGCGGCCTGGAAGTCAGCTTCGAAGAACTGCGCACCTTCGGTGGCCATGACGCTAAATTCATTGAC
AGCTTGCAAGAGAATGAGTTCCGTCTGTACTACTATAACAAATTCAAAGACATTGCAAGCACGTTGAACAAGGCC
AAAAGCATCGTTGGTACTACCGCGTCGTTGCAGTATATGAAGAATGTGTTTAAAGAGAAGTACCTGCTGTCCGAG
GATACCTCCGGCAAGTTTAGCGTTGATAAGCTGAAGTTTGACAAACTGTACAAGATGCTGACCGAGATTTACACC
GAGGACAACTTTGTGAAATTCTTCAAAGTGTTGAATCGTAAAACCTATCTGAATTTTGACAAAGCGGTTTTCAAG
ATTAACATCGTGCCGAAGGTGAACTACACCATCTATGACGGTTTTAACCTGCGTAACACCAACCTGGCGGCGAAC
TTTAACGGTCAGAATACGGAAATCAACAACATGAATTTCACGAAGTTGAAGAACTTCACGGGTCTGTTCGAGTTC
TATAAGCTGCTGTGCGTGCGCGGTATCATCACCAGCAAAACCAAAAGCCTGGACAAAGGCTACAACAAGGCGCTG
AATGACCTGTGCATTAAGGTAAACAATTGGGATCTGTTCTTTTCGCCATCCGAAGATAATTTTACCAACGACCTG
AACAAGGGTGAAGAAATCACCAGCGATACGAATATTGAAGCAGCGGAAGAGAATATCAGCCTGGATCTGATCCAG
CAGTACTATCTGACCTTTAACTTCGACAATGAACCGGAGAACATTAGCATTGAGAATCTGAGCAGCGACATTATC
GGTCAGCTGGAACTGATGCCGAATATCGAACGTTTCCCGAACGGCAAAAAGTACGAGCTGGACAAGTACACTATG
TTCCATTACCTGCGTGCACAGGAGTTTGAACACGGTAAAAGCCGTATCGCGCTGACCAACAGCGTTAACGAGGCC
CTGCTGAACCCGAGCCGTGTCTATACCTTCTTCAGCAGCGACTATGTTAAGAAAGTGAACAAAGCCACTGAGGCC
GCGATGTTCCTGGGCTGGGTGGAACAGCTGGTATATGACTTCACGGACGAGACGAGCGAAGTGAGCACTACCGAC
AAAATTGCTGATATTACCATCATTATCCCGTATATTGGTCCGGCACTGAACATTGGCAACATGCTGTACAAAGAC
GATTTTGTGGGTGCCCTGATCTTCTCCGGTGCCGTGATTCTGCTGGAGTTCATTCCGGAGATTGCGATCCCGGTG
TTGGGTACCTTCGCGCTGGTGTCCTACATCGCGAATAAGGTTCTGACGGTTCAGACCATCGATAACGCGCTGTCG
AAACGTAATGAAAAATGGGACGAGGTTTACAAATACATTGTTACGAATTGGCTGGCGAAAGTCAATACCCAGATC
GACCTGATCCGTAAGAAAATGAAAGAGGCGCTGGAGAATCAGGCGGAGGCCACCAAAGCAATTATCAACTACCAA
TACAACCAGTACACGGAAGAAGAGAAGAATAACATTAACTTCAATATCGATGATTTGAGCAGCAAGCTGAATGAA
TCTATCAACAAAGCGATGATCAATATCAACAAGTTTTTGAATCAGTGTAGCGTTTCGTACCTGATGAATAGCATG
ATTCCGTATGGCGTCAAACGTCTGGAGGACTTCGACGCCAGCCTGAAAGATGCGTTGCTGAAATACATTTACGAC
AATCGTGGTACGCTGATTGGCCAAGTTGACCGCTTGAAAGACAAAGTTAACAATACCCTGAGCACCGACATCCCA
TTTCAACTGAGCAAGTATGTTGATAATCAACGTCTGTTGAGCACTTTCACCGAGTATATCAAAAACATCATCAAT
ACTAGCATTCTGAACCTGCGTTACGAGAGCAATCATCTGATTGATCTGAGCCGTTATGCAAGCAAGATCAACATC
GGTAGCAAGGTCAATTTTGACCCGATCGATAAGAACCAGATCCAGCTGTTTAATCTGGAATCGAGCAAAATTGAG
GTTATCCTGAAAAACGCCATTGTCTACAACTCCATGTACGAGAATTTCTCCACCAGCTTCTGGATTCGCATCCCG
AAATACTTCAACAGCATTAGCCTGAACAACGAGTATACTATCATCAACTGTATGGAGAACAACAGCGGTTGGAAG
GTGTCTCTGAACTATGGTGAGATCATTTGGACCTTGCAGGACACCCAAGAGATCAAGCAGCGCGTCGTGTTCAAG
TACTCTCAAATGATCAACATTTCCGATTACATTAATCGTTGGATCTTCGTGACCATTACGAATAACCGTCTGAAT
AACAGCAAGATTTACATCAATGGTCGCTTGATCGATCAGAAACCGATTAGCAACCTGGGTAATATCCACGCAAGC
AACAACATTATGTTCAAATTGGACGGTTGCCGCGATACCCATCGTTATATCTGGATCAAGTATTTCAACCTGTTT
GATAAAGAACTGAATGAGAAGGAGATCAAAGATTTGTATGACAACCAATCTAACAGCGGCATTTTGAAGGACTTC
TGGGGCGATTATCTGCAATACGATAAGCCGTACTATATGCTGAACCTGTATGATCCGAACAAATATGTGGATGTC
AATAATGTGGGTATTCGTGGTTACATGTATTTGAAGGGTCCGCGTGGCAGCGTTATGACGACCAACATTTACCTG
AACTCTAGCCTGTACCGTGGTACGAAATTCATCATTAAGAAATATGCCAGCGGCAACAAAGATAACATTGTGCGT
AATAACGATCGTGTCTACATCAACGTGGTCGTGAAGAATAAAGAGTACCGTCTGGCGACCAACGCTTCGCAGGCG
GGTGTTGAGAAAATTCTGAGCGCGTTGGAGATCCCTGATGTCGGTAATCTGAGCCAAGTCGTGGTTATGAAGAGC
AAGAACGACCAGGGTATCACTAACAAGTGCAAGATGAACCTGCAAGACAACAATGGTAACGACATCGGCTTTATT
GGTTTCCACCAGTTCAACAATATTGCTAAACTGGTAGCGAGCAATTGGTACAATCGTCAGATTGAGCGCAGCAGC
CGTACTTTGGGCTGTAGCTGGGAGTTTATCCCGGTCGATGATGGTTGGGGCGAACGTCCGCTG
SEQ ID NO: 2
(Polypeptide Sequence of Unmodified BoNT/A)
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVFK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNIINTSILNLRYESNHLIDLSRYASKINI
GSKVNFDPIDKNQIQLFNLESSKIEVILKNAIVYNSMYENFSTSFWIRIPKYFNSISLNNEYTIINCMENNSGWK
VSLNYGEIIWTLQDTQEIKQRVVFKYSQMINISDYINRWIFVTITNNRLNNSKIYINGRLIDQKPISNLGNIHAS
NNIMFKLDGCRDTHRYIWIKYFNLFDKELNEKEIKDLYDNQSNSGILKDFWGDYLQYDKPYYMLNLYDPNKYVDV
NNVGIRGYMYLKGPRGSVMTTNIYLNSSLYRGTKFIIKKYASGNKDNIVRNNDRVYINVVVKNKEYRLATNASQA
GVEKILSALEIPDVGNLSQVVVMKSKNDQGITNKCKMNLQDNNGNDIGFIGFHQFNNIAKLVASNWYNRQIERSS
RTLGCSWEFIPVDDGWGERPL
SEQ ID NO: 3
(Polypeptide Sequence of Modified BoNT/A “Chimera 1”)
MPFVNKQFNYKDPVNGVDIAYIKIPNAGQMQPVKAFKIHNKIWVIPERDTFTNPEEGDLN
PPPEAKQVPVSYYDSTYLSTDNEKDNYLKGVTKLFERIYSTDLGRMLLTSIVRGIPFWGG
STIDTELKVIDTNCINVIQPDGSYRSEELNLVIIGPSADIIQFECKSFGHEVLNLTRNGY
GSTQYIRFSPDFTFGFEESLEVDTNPLLGAGKFATDPAVTLAHELIHAGHRLYGIAINPN
RVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKEDKLYKMLTEIYTEDNFVKFFKV
LNRKTYLNFDKAVFKINIVPKVNYTIYDGFNLRNTNLAANFNGQNTEINNMNFTKLKNFT
GLFEFYKLLCVRGIITSKTKSLDKGYNKALNDLCIKVNNWDLFFSPSEDNFTNDLNKGEE
ITSDTNIEAAEENISLDLIQQYYLTFNFDNEPENISIENLSSDIIGQLELMPNIERFPNG
KKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSG
AVILLEFIPEIAIPVLGTFALVSYIANKVLTVQTIDNALSKRNEKWDEVYKYIVTNWLAK
VNTQIDLIRKKMKEALENQAEATKAIINYQYNQYTEEEKNNINFNIDDLSSKLNESINKA
MININKFLNQCSVSYLMNSMIPYGVKRLEDFDASLKDALLKYIYDNRGTLIGQVDRLKDK
VNNTLSTDIPFQLSKYVDNQRLLSTFTEYIKSEILNNIILNLRYKDNNLIDLSGYGAKVE
VYDGVELNDKNQFKLTSSANSKIRVTQNQNIIFNSVFLDFSVSFWIRIPKYKNDGIQNYI
HNEYTIINCMKNNSGWKISIRGNRIIWTLIDINGKTKSVFFEYNIREDISEYINRWFFVT
ITNNLNNAKIYINGKLESNTDIKDIREVIANGEIIFKLDGDIDRTQFIWMKYESIFNTEL
SQSNIEERYKIQSYSEYLKDFWGNPLMYNKEYYMFNAGNKNSYIKLKKDSPVGEILTRSK
YNQNSKYINYRDLYIGEKFIIRRKSNSQSINDDIVRKEDYIYLDFFNLNQEWRVYTYKYF
KKEEMKLFLAPIYDSDEFYNTIQIKEYDEQPTYSCQLLFKKDEESTDEIGLIGIHRFYES
GIVFEEYKDYFCISKWYLKEVKRKPYNLKLGCNWQFIPKDEGWTEHHHHHHHHHH
SEQ ID NO: 4
(Polypeptide Sequence of Modified BoNT/A “Chimera 2”)
MPFVNKQFNYKDPVNGVDIAYIKIPNAGQMQPVKAFKIHNKIWVIPERDTFTNPEEGDLN
PPPEAKQVPVSYYDSTYLSTDNEKDNYLKGVTKLFERIYSTDLGRMLLTSIVRGIPFWGG
STIDTELKVIDTNCINVIQPDGSYRSEELNLVIIGPSADIIQFECKSFGHEVLNLTRNGY
GSTQYIRFSPDFTFGFEESLEVDTNPLLGAGKFATDPAVTLAHELIHAGHRLYGIAINPN
RVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKV
LNRKTYLNFDKAVFKINIVPKVNYTIYDGFNLRNTNLAANFNGQNTEINNMNFTKLKNFT
GLFEFYKLLCVRGIITSKTKSLDKGYNKALNDLCIKVNNWDLFFSPSEDNFTNDLNKGEE
ITSDTNIEAAEENISLDLIQQYYLTFNFDNEPENISIENLSSDIIGQLELMPNIERFPNG
KKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSG
AVILLEFIPEIAIPVLGTFALVSYIANKVLTVQTIDNALSKRNEKWDEVYKYIVTNWLAK
VNTQIDLIRKKMKEALENQAEATKAIINYQYNQYTEEEKNNINENIDDLSSKLNESINKA
MININKFLNQCSVSYLMNSMIPYGVKRLEDFDASLKDALLKYIYDNRGTLIGQVDRLKDK
VNNTLSTDIPFQLSKYVDNQRLLSTFTEYIKNIIELGGGGSELSEILNNIILNLRYKDNN
LIDLSGYGAKVEVYDGVELNDKNQFKLTSSANSKIRVTQNQNIIFNSVFLDFSVSFWIRI
PKYKNDGIQNYIHNEYTIINCMKNNSGWKISIRGNRIIWTLIDINGKTKSVFFEYNIRED
ISEYINRWFFVTITNNLNNAKIYINGKLESNTDIKDIREVIANGEIIFKLDGDIDRTQFI
WMKYFSIFNTELSQSNIEERYKIQSYSEYLKDFWGNPLMYNKEYYMFNAGNKNSYIKLKK
DSPVGEILTRSKYNQNSKYINYRDLYIGEKFIIRRKSNSQSINDDIVRKEDYIYLDFFNL
NQEWRVYTYKYFKKEEMKLFLAPIYDSDEFYNTIQIKEYDEQPTYSCQLLFKKDEESTDE
IGLIGIHRFYESGIVFEEYKDYFCISKWYLKEVKRKPYNLKLGCNWQFIPKDEGWTEHHH
HHHHHHH
SEQ ID NO: 5
(Polypeptide Sequence of Modified BoNT/A “Chimera 3A”)
MPFVNKQFNYKDPVNGVDIAYIKIPNAGQMQPVKAFKIHNKIWVIPERDTFTNPEEGDLN
PPPEAKQVPVSYYDSTYLSTDNEKDNYLKGVTKLFERIYSTDLGRMLLTSIVRGIPFWGG
STIDTELKVIDTNCINVIQPDGSYRSEELNLVIIGPSADIIQFECKSFGHEVLNLTRNGY
GSTQYIRFSPDFTFGFEESLEVDTNPLLGAGKFATDPAVTLAHELIHAGHRLYGIAINPN
RVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKV
LNRKTYLNFDKAVFKINIVPKVNYTIYDGFNLRNTNLAANFNGQNTEINNMNFTKLKNFT
GLFEFYKLLCVRGIITSKTKSLDKGYNKALNDLCIKVNNWDLFFSPSEDNFTNDLNKGEE
ITSDTNIEAAEENISLDLIQQYYLTFNFDNEPENISIENLSSDIIGQLELMPNIERFPNG
KKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSG
AVILLEFIPEIAIPVLGTFALVSYIANKVLTVQTIDNALSKRNEKWDEVYKYIVTNWLAK
VNTQIDLIRKKMKEALENQAEATKAIINYQYNQYTEEEKNNINFNIDDLSSKLNESINKA
MININKFLNQCSVSYLMNSMIPYGVKRLEDFDASLKDALLKYIYDNRGTLIGQVDRLKDK
VNNTLSTDIPFQLSKYVDNQRLLSTFTEYIKNILNNIILNLRYKDNNLIDLSGYGAKVEV
YDGVELNDKNQFKLTSSANSKIRVTQNQNIIFNSVFLDFSVSFWIRIPKYKNDGIQNYIH
NEYTIINCMKNNSGWKISIRGNRIIWTLIDINGKTKSVFFEYNIREDISEYINRWFFVTI
TNNLNNAKIYINGKLESNTDIKDIREVIANGEIIFKLDGDIDRTQFIWMKYFSIFNTELS
QSNIEERYKIQSYSEYLKDFWGNPLMYNKEYYMFNAGNKNSYIKLKKDSPVGEILTRSKY
NQNSKYINYRDLYIGEKFIIRRKSNSQSINDDIVRKEDYIYLDFFNLNQEWRVYTYKYFK
KEEMKLFLAPIYDSDEFYNTIQIKEYDEQPTYSCQLLFKKDEESTDEIGLIGIHRFYESG
IVFEEYKDYFCISKWYLKEVKRKPYNLKLGCNWQFIPKDEGWTEHHHHHHHHHH
SEQ ID NO: 6
(Polypeptide Sequence of Modified BoNT/A “Chimera 3B”)
MPFVNKQFNYKDPVNGVDIAYIKIPNAGQMQPVKAFKIHNKIWVIPERDTFTNPEEGDLNPPPEAKQVPVSYYDS
TYLSTDNEKDNYLKGVTKLFERIYSTDLGRMLLTSIVRGIPFWGGSTIDTELKVIDTNCINVIOPDGSYRSEELN
LVIIGPSADIIQFECKSFGHEVLNLTRNGYGSTQYIRFSPDFTFGFEESLEVDTNPLLGAGKFATDPAVTLAHEL
IHAGHRLYGIAINPNRVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKVLNRKTYLNFDKAVEK
INIVPKVNYTIYDGFNLRNTNLAANFNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQQYYLTFNFDNEPENISIENLSSDII
GQLELMPNIERFPNGKKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSGAVILLEFIPEIAIPV
LGTFALVSYIANKVLTVQTIDNALSKRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSMIPYGVKRLEDFDASLKDALLKYIYD
NRGTLIGQVDRLKDKVNNTLSTDIPFQLSKYVDNQRLLSTFTEYIKNILNNIILNLRYKDNNLIDLSGYGAKVEV
YDGVELNDKNQFKLTSSANSKIRVTQNQNIIFNSVFLDFSVSFWIRIPKYKNDGIQNYIHNEYTIINCMKNNSGW
KISIRGNRIIWTLIDINGKTKSVFFEYNIREDISEYINRWFFVTITNNLNNAKIYINGKLESNTDIKDIREVIAN
GEIIFKLDGDIDRTQFIWMKYFSIFNTELSQSNIEERYKIQSYSEYLKDFWGNPLMYNKEYYMFNAGNKNSYIKL
KKDSPVGEILTRSKYNQNSKYINYRDLYIGEKFIIRRKSNSQSINDDIVRKEDYIYLDFFNLNQEWRVYTYKYFK
KEEMKLFLAPIYDSDEFYNTIQIKEYDEQPTYSCOLLFKKDEESTDEIGLIGIHRFYESGIVFEEYKDYFCISKW
YLKEVKRKPYNLKLGCNWQFIPKDEGWTE
SEQ ID NO: 7
(Polypeptide Sequence of Modified BoNT/A “Chimera 3C”)
MPFVNKQFNYKDPVNGVDIAYIKIPNAGQMQPVKAFKIHNKIWVIPERDTFTNPEEGDLN
PPPEAKQVPVSYYDSTYLSTDNEKDNYLKGVTKLFERIYSTDLGRMLLTSIVRGIPFWGG
STIDTELKVIDTNCINVIQPDGSYRSEELNLVIIGPSADIIQFECKSFGHEVLNLTRNGY
GSTQYIRFSPDFTFGFEESLEVDTNPLLGAGKFATDPAVTLAHELIHAGHRLYGIAINPN
RVFKVNTNAYYEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYTEDNFVKFFKV
LNRKTYLNFDKAVFKINIVPKVNYTIYDGFNLRNTNLAANFNGQNTEINNMNFTKLKNFT
GLFEFYKLLCVRGIITSKTKSLDKGYNKALNDLCIKVNNWDLFFSPSEDNFTNDLNKGEE
ITSDTNIEAAEENISLDLIQQYYLTFNFDNEPENISIENLSSDIIGQLELMPNIERFPNG
KKYELDKYTMFHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKDDFVGALIFSG
AVILLEFIPEIAIPVLGTFALVSYIANKVLTVQTIDNALSKRNEKWDEVYKYIVTNWLAK
VNTQIDLIRKKMKEALENQAEATKAIINYQYNQYTEEEKNNINFNIDDLSSKLNESINKA
MININKFLNQCSVSYLMNSMIPYGVKRLEDFDASLKDALLKYIYDNRGTLIGQVDRLKDK
VNNTLSTDIPFQLSKYVDNQRLLSTFTEYIKNILNNIILNLRYKDNNLIDLSGYGAKVEV
YDGVELNDKNQFKLTSSANSKIRVTQNQNIIFNSVFLDFSVSFWIRIPKYKNDGIQNYIH
NEYTIINCMKNNSGWKISIRGNRIIWTLIDINGKTKSVFFEYNIREDISEYINRWFFVTI
TNNLNNAKIYINGKLESNTDIKDIREVIANGEIIFKLDGDIDRTQFIWMKYFSIFNTELS
QSNIEERYKIQSYSEYLKDFWGNPLMYNKEYYMFNAGNKNSYIKLKKDSPVGEILTRSKY
NQNSKYINYRDLYIGEKFIIRRKSNSQSINDDIVRKEDYIYLDFFNLNQEWRVYTYKYFK
KEEEKLFLAPISDSDEFYNTIQIKEYDEQPTYSCOLLFKKDEESTDEIGLIGIHRFYESG
IVFEEYKDYFCISKWYLKEVKRKPYNLKLGCNWQFIPKDEGWTE
SEQ ID NO: 8
(Polypeptide Sequence of BoNT/B)
MPVTINNFNYNDPIDNNNIIMMEPPFARGTGRYYKAFKITDRIWIIPERYTFGYKPEDFN
KSSGIFNRDVCEYYDPDYLNTNDKKNIFLQTMIKLFNRIKSKPLGEKLLEMIINGIPYLG
DRRVPLEEFNTNIASVTVNKLISNPGEVERKKGIFANLIIFGPGPVLNENETIDIGIQNH
FASREGFGGIMQMKFCPEYVSVFNNVQENKGASIFNRRGYFSDPALILMHELIHVLHGLY
GIKVDDLPIVPNEKKFFMQSTDAIQAEELYTFGGQDPSIITPSTDKSIYDKVLQNFRGIV
DRLNKVLVCISDPNININIYKNKFKDKYKFVEDSEGKYSIDVESFDKLYKSLMFGFTETN
IAENYKIKTRASYFSDSLPPVKIKNLLDNEIYTIEEGFNISDKDMEKEYRGQNKAINKQA
YEEISKEHLAVYKIQMCKSVKAPGICIDVDNEDLFFIADKNSFSDDLSKNERIEYNTQSN
YIENDFPINELILDTDLISKIELPSENTESLTDFNVDVPVYEKQPAIKKIFTDENTIFQY
LYSQTFPLDIRDISLTSSFDDALLFSNKVYSFFSMDYIKTANKVVEAGLFAGWVKQIVND
FVIEANKSNTMDKIADISLIVPYIGLALNVGNETAKGNFENAFEIAGASILLEFIPELLI
PVVGAFLLESYIDNKNKIIKTIDNALTKRNEKWSDMYGLIVAQWLSTVNTQFYTIKEGMY
KALNYQAQALEEIIKYRYNIYSEKEKSNINIDFNDINSKLNEGINQAIDNINNFINGCSV
SYLMKKMIPLAVEKLLDFDNTLKKNLLNYIDENKLYLIGSAEYEKSKVNKYLKTIMPEDL
SIYTNDTILIEMFNKYNSEILNNIILNLRYKDNNLIDLSGYGAKVEVYDGVELNDKNQFK
LTSSANSKIRVTQNQNIIFNSVFLDFSVSFWIRIPKYKNDGIQNYIHNEYTIINCMKNNS
GWKISIRGNRIIWTLIDINGKTKSVFFEYNIREDISEYINRWFFVTITNNLNNAKIYING
KLESNTDIKDIREVIANGEIIFKLDGDIDRTQFIWMKYFSIFNTELSQSNIEERYKIQSY
SEYLKDFWGNPLMYNKEYYMFNAGNKNSYIKLKKDSPVGEILTRSKYNQNSKYINYRDLY
IGEKFIIRRKSNSQSINDDIVRKEDYIYLDFENLNQEWRVYTYKYFKKEEEKLFLAPISD
SDEFYNTIQIKEYDEQPTYSCQLLFKKDEESTDEIGLIGIHRFYESGIVFEEYKDYFCIS
KWYLKEVKRKPYNLKLGCNWQFIPKDEGWTE
SEQ ID NO: 9
C-terminal L-chain Fragment
TKSLDKGYNK
SEQ ID NO: 10
C-terminal L-chain Fragment 2
SLDKGYNK
SEQ ID NO: 11
Di-Chain L-Chain 1
PFVNKQFNYKDPVNGVDIAYIKIPNAGQMQPVKAFKIHNKIWVIPERDTFTNPEEGDLNPPPEAKQVPVSYYDST
YLSTDNEKDNYLKGVTKLFERIYSTDLGRMLLTSIVRGIPFWGGSTIDTELKVIDTNCINVIQPDGSYRSEELNL
VIIGPSADIIQFECKSFGHEVLNLTRNGYGSTQYIRFSPDFTFGFEESLEVDTNPLLGAGKFATDPAVTLAHELI
HAGHRLYGIAINPNRVFKVNTNAYYEMSGLEVSFEELRTFGGHDAKFIDSLQENEFRLYYYNKFKDIASTLNKAK
SIVGTTASLQYMKNVFKEKYLLSEDTSGKFSVDKLKFDKLYKMLTEIYTEDNFVKFFKVLNRKTYLNFDKAVFKI
NIVPKVNYTIYDGFNLRNTNLAANFNGQNTEINNMNFTKLKNFTGLFEFYKLLCVRGIITSK
SEQ ID NO: 12
Di-Chain L-Chain 2
PFVNKQFNYKDPVNGVDIAYIKIPNAGQMQPVKAFKIHNKIWVIPERDTFTNPEEGDLNPPPEAKQVPVSYYDST
YLSTDNEKDNYLKGVTKLFERIYSTDLGRMLLTSIVRGIPFWGGSTIDTELKVIDTNCINVIQPDGSYRSEELNL
VIIGPSADIIQFECKSFGHEVLNLTRNGYGSTQYIRFSPDFTFGFEESLEVDTNPLLGAGKFATDPAVTLAHELI
HAGHRLYGIAINPNRVFKVNTNAYYEMSGLEVSFEELRTFGGHDAKFIDSLQENEFRLYYYNKFKDIASTLNKAK
SIVGTTASLQYMKNVFKEKYLLSEDTSGKFSVDKLKFDKLYKMLTEIYTEDNFVKFFKVLNRKTYLNFDKAVFKI
NIVPKVNYTIYDGFNLRNTNLAANFNGQNTEINNMNFTKLKNFTGLFEFYKLLCVRGIITSKTK
SEQ ID NO: 13
Di-Chain H-Chain
ALNDLCIKVNNWDLFFSPSEDNFTNDLNKGEEITSDTNIEAAEENISLDLIQQYYLTFNFDNEPENISIENLSSD
IIGQLELMPNIERFPNGKKYELDKYTMFHYLRAQEFEHGKSRIALTNSVNEALLNPSRVYTFFSSDYVKKVNKAT
EAAMFLGWVEQLVYDFTDETSEVSTTDKIADITIIIPYIGPALNIGNMLYKDDFVGALIFSGAVILLEFIPEIAI
PVLGTFALVSYIANKVLTVQTIDNALSKRNEKWDEVYKYIVTNWLAKVNTQIDLIRKKMKEALENQAEATKAIIN
YQYNQYTEEEKNNINFNIDDLSSKLNESINKAMININKFLNQCSVSYLMNSMIPYGVKRLEDFDASLKDALLKYI
YDNRGTLIGQVDRLKDKVNNTLSTDIPFQLSKYVDNQRLLSTFTEYIKNILNNIILNLRYKDNNLIDLSGYGAKV
EVYDGVELNDKNQFKLTSSANSKIRVTQNQNIIFNSVFLDFSVSFWIRIPKYKNDGIQNYIHNEYTIINCMKNNS
GWKISIRGNRIIWTLIDINGKTKSVFFEYNIREDISEYINRWFFVTITNNLNNAKIYINGKLESNTDIKDIREVI
ANGEIIFKLDGDIDRTQFIWMKYFSIFNTELSQSNIEERYKIQSYSEYLKDFWGNPLMYNKEYYMFNAGNKNSYI
KLKKDSPVGEILTRSKYNQNSKYINYRDLYIGEKFIIRRKSNSQSINDDIVRKEDYIYLDFFNLNQEWRVYTYKY
FKKEEMKLFLAPIYDSDEFYNTIQIKEYDEQPTYSCQLLFKKDEESTDEIGLIGIHRFYESGIVFEEYKDYFCIS
KWYLKEVKRKPYNLKLGCNWQFIPKDEGWTE

### EXAMPLES

### Example 1

### Cloning, Expression and Purification of BoNT/AB Chimeras

BoNT/AB chimeric constructs 1, 2, 3A, 3B, and 3C (SEQ ID NO: 3 to 7, respectively) were constructed from DNA encoding the parent serotype molecule and appropriate oligonucleotides using standard molecular biology techniques. These were then cloned into the pJ401 expression vector with or without a C-terminal His₁₀-tag and transformed into BLR (DE3) E. coli cells for over-expression. These cells were grown at 37° C. and 225 RPM shaking in 2 L baffled conical flasks containing 1 L modified Terrific Broth (mTB) supplemented with the appropriate antibiotic. Once the A₆₀₀ reached >0.5, the incubator temperature was decreased to 16° C., and then induced with 1 mM IPTG an hour later for 20 h at 225 RPM shaking, to express the recombinant BoNT/AB construct.

Harvested cells were lysed by ultrasonication and clarified by centrifugation at 4500 RPM for 1 h at 4° C. The recombinant BoNT/AB chimeric molecules were then extracted in ammonium sulphate and purified by standard fast protein liquid chromatography (FPLC) techniques. This involved using a hydrophobic interaction resin for capture and an anion-exchange resin for the intermediate purification step. The partially purified molecules were then proteolytically cleaved with endoproteinase Lys-C to yield the active di-chain. This was further purified with a second hydrophobic interaction resin to obtain the final BoNT/AB chimera.

For BoNT/AB chimeric molecules with a decahistadine tag (H₁₀) (chimera 1, 2, 3A), the capture step employed the use of an immobilised nickel resin instead of the hydrophobic interaction resin.

The sequence of each chimera is presented in Table 1.

TABLE 1
chimeric BoNT/AB constructs

| | SEQ | |
|---|---|---|
| | ID | |
| Molecule | NO | Sequence |
| Chimera 1 | 3 | A1: 1-871 + B1: 858-1291 (E1191M/S1199Y) + |
| | | His₁₀-tag |
| Chimera 2 | 4 | A1: 1-874 + ELGGGGSEL + B1: 858-1291 |
| | | (E1191M/S1199Y) + His₁₀-tag |
| Chimera 3A | 5 | A1: 1-872 + B1: 860-1291 (E1191M/S1199Y) + |
| | | His₁₀-tag |
| Chimera 3B | 6 | A1: 1-872 + B1: 860-1291 (E1191M/S1199Y) |
| Chimera 3C | 7 | A1: 1-872 + B1: 860-1291 |

### Example 2

### Comparison of BoNT/AB Chimera 1, 2 and 3A

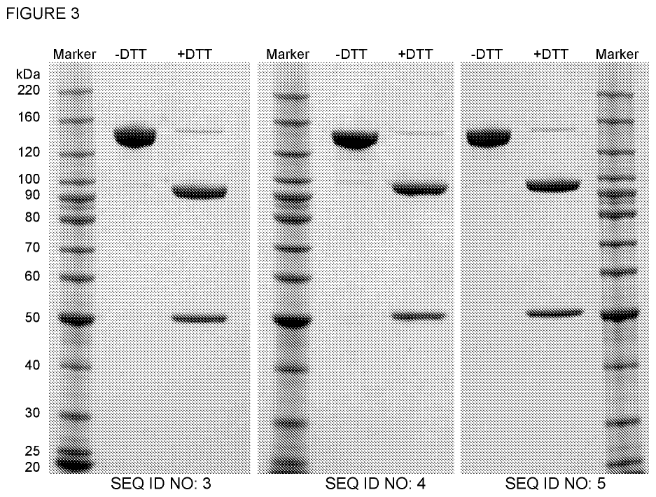

BoNT/AB chimera 1, 2 and 3A which have a C-terminal His₁₀ tag and E1191M/S1199Y double mutation were purified as described in Example 1 (FIG. 3) and tested for functional activity.

### Rat Spinal Cord Neurons Snap-25 Cleavage Assay

Primary cultures of rat spinal cord neurons (SCN) were prepared and grown, for 3 weeks, in 96 well tissue culture plates (as described in: Masuyer et al., 2011, J. Struct. Biol. Structure and activity of a functional derivative of Clostridium botulinum neurotoxin B; and in: Chaddock et al., 2002, Protein Expr. Purif. Expression and purification of catalytically active, non-toxic endopeptidase derivatives of Clostridium botulinum toxin type A). Serial dilutions of BoNT/AB were prepared in SCN feeding medium. The growth medium from the wells to be treated was collected and filtered (0.2 m filter). 125 L of the filtered medium was added back to each test well. 125 L of diluted toxin was then added to the plate (triplicate wells). The treated cells were incubated at 37° C., 10% CO₂, for 24±1 h).

### Analysis of BoNT Activity Using the SNAP-25 Cleavage Assay

Following treatment, BoNT was removed and cells were washed once in PBS (Gibco, UK). Cells were lysed in 1× NuPAGE lysis buffer (Life Technologies) supplemented with 0.1 M dithiothreitol (DTT) and 250 units/mL benzonase (Sigma). Lysate proteins were separated by SDS-PAGE and transferred to nitrocellulose membranes. Membranes were probed with a primary antibody specific for SNAP-25 (Sigma #S9684) which recognizes uncleaved SNAP-25 as well as SNAP-25 cleaved by the BoNT/A endopeptidase. The secondary antibody used was an HRP-conjugated anti-rabbit IgG (Sigma #A6154). Bands were detected by enhanced chemiluminescence and imaged using a pXi6 Access (Synoptics, UK). The intensity of bands was determined using GeneTools software (Syngene, Cambridge, UK) and the percentage of SNAP-25 cleaved at each concentration of BoNT calculated. Data were fitted to a 4-parameter logistic equation and pEC₅₀ calculated using GraphPad Prism version 6 (GraphPad).

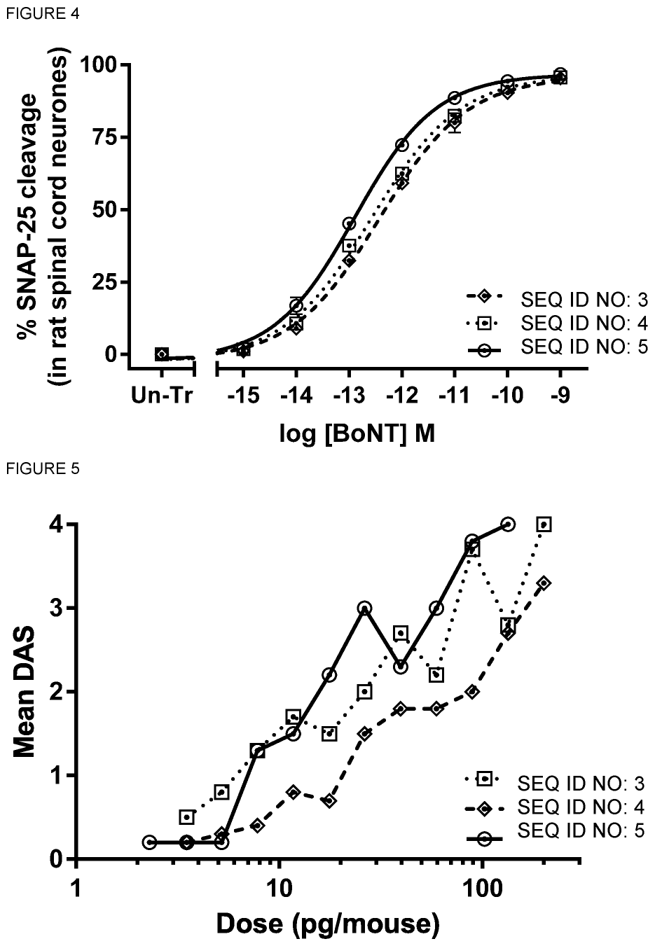

Table 2 below provides the pEC₅₀ values determined for Chimera 1, 2 and 3A in the rat SCN SNAP-25 cleavage assay. These results show that the three BoNT/AB chimeras retained the ability to enter rat spinal cord neurons and cleave their target substrate. However, chimera 3A was more potent than chimera 1 and 2 in this assay (see also FIG. 4).

TABLE 2
pEC₅₀ values.

| | pEC₅₀ ± SEM |
|---|---|
| Chimera 1 | 12.42 ± 0.04 |
| Chimera 2 | 12.57 ± 0.01 |
| Chimera 3A | 12.89 ± 0.04 |

### Digit Abduction Scoring (DAS) Assay

The method to measure the activity of BoNT/AB chimera 1, 2 and 3A in the DAS assay is based on the startled response toe spreading reflex of mice, when suspended briefly by the tail. This reflex is scored as Digit Abduction Score (DAS) and is inhibited after administration of BoNT into the gastrocnemius-soleus muscles of the hind paw. Mice are suspended briefly by the tail to elicit a characteristic startled response in which the animal extends its hind limb and abducts its hind digits. (Aoki et al. 1999, Eur. J. Neurol.; 6 (suppl. 4) S3-S10).

On the day of injection, mice were anaesthetized in an induction chamber receiving isoflurane 3% in oxygen. Each mouse received an intramuscular injection of BoNT/AB chimera or vehicle (phosphate buffer containing 0.2% gelatine) in the gastrocnemius-soleus muscles of the right hind paw.

Following neurotoxin injection, the varying degrees of digit abduction were scored on a scale from zero to four, where 0=normal and 4=maximal reduction in digit abduction and leg extension. ED50 was determined by nonlinear adjustment analysis using average of maximal effect at each dose. The mathematical model used was the 4 parameters logistic model.

DAS was performed every 2 hours during the first day after dosing; thereafter it was performed 3 times a day for 4 days.

FIG. 5 shows the fitted curves for chimera 1, 2 and 3A (SEQ ID NO: 3, 4 and 5 converted into a di-chain form, respectively). The chimera 3A curve is shifted to the left, meaning lower doses of chimera 3A achieved a similar DAS response compared to chimera 1 and 2, therefore showing that chimera 3A is more potent than the others in the mouse DAS assay; see also the table below (Table 3) that provides the values for the calculated ED50 and the dose leading to DAS 4 (highest score) for each chimera.

Table 3 below provides the ED₅₀ and DAS 4 doses determined for unmodified recombinant BoNT/A1 (rBoNT/A1—SEQ ID NO: 2 converted into a di-chain form) and chimeras 1, 2 and 3A in the mouse DAS assay. These results show that of the three chimeras, chimera 3A has the highest in vivo potency in inducing muscle weakening. Studies shown in FIG. 5 and Table 3 were performed in mice obtained from Charles River laboratories.

TABLE 3
ED₅₀ values.

| | ED₅₀ | DAS 4 dose |
|---|---|---|
| | (pg/mouse) | (pg/mouse) |
| rBoNT/A1 | 1 | 5 |
| Chimera 1 | 23 | 200 |
| Chimera 2 | 89 | >300 |
| Chimera 3A | 18 | 133 |

### Example 3

### Comparison of BoNT/AB Chimera 3B, 3C and Unmodified BoNT/A1

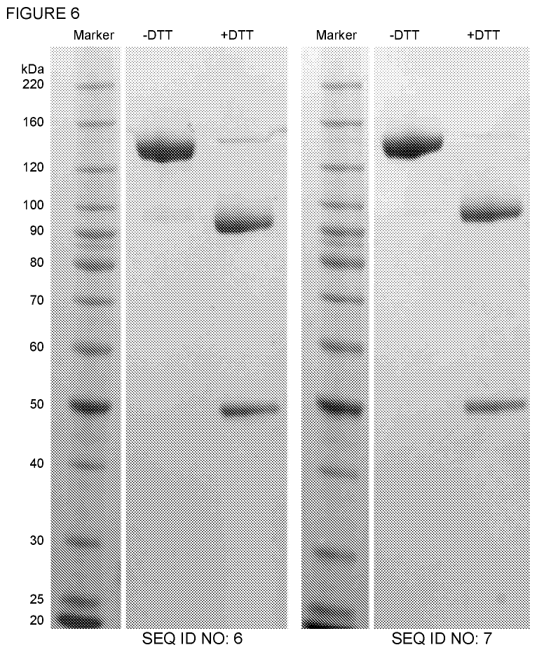

Untagged BoNT/AB chimera 3B and 3C, respectively with and without the presence of the E1191M/S1199Y double mutation (SEQ ID NO: 6 and 7) were purified as described in Example 1 (FIG. 6), and tested for functional activity using unmodified BoNT/A (SEQ ID NO: 2 converted into a di-chain form) as a reference.

### Human Pluripotent Stem Cells Snap-25 Cleavage Assay

Cryopreserved PERI.4U-cells were purchased from Axiogenesis (Cologne, Germany). Thawing and plating of the cells were performed as recommended by the manufacturer. Briefly, cryovials containing the cells were thawed in a water bath at 37° C. for 2 minutes. After gentle resuspension the cells were transferred to a 50 mL tube. The cryovial was washed with 1 mL of Peri.4U® thawing medium supplied by the manufacturer and the medium was transferred drop-wise to the cell suspension to the 50 mL tube, prior to adding a further 2 mL of Peri.4U® thawing medium drop-wise to the 50 mL tube. Cells were then counted using a hemocytometer. After this, a further 6 mL of Peri.4U® thawing medium was added to the cell suspension. A cell pellet was obtained by centrifugation at 260×g (e.g. 1,100 RPM) for 6 minutes at room temperature. Cells were then resuspended in complete Peri.4U® culture medium supplied by the manufacturer. Cells were plated at a density of 50,000 to 150,000 cells per cm² on cell culture plates coated with poly-L-ornithine and laminin. Cells were cultured at 37° C. in a humidified CO₂ atmosphere, and medium was changed completely every 2-3 days during culture.

For toxin treatment, serial dilutions of BoNTs were prepared in Peri.4U® culture medium. The medium from the wells to be treated was collected and filtered (0.2 m filter). 125 L of the filtered medium was added back to each test well. 125 L of diluted toxin was then added to the plate (triplicate wells). The treated cells were incubated at 37° C., 10% CO₂, for 48±1 h).

### Analysis of BoNT Activity Using the SNAP-25 Cleavage Assay

Following treatment, BoNT was removed and cells were washed once in PBS (Gibco, UK). Cells were lysed in 1× NuPAGE lysis buffer (Life Technologies) supplemented with 0.1 M dithiothreitol (DTT) and 250 units/mL benzonase (Sigma). Lysate proteins were separated by SDS-PAGE and transferred to nitrocellulose membranes. Membranes were probed with a primary antibody specific for SNAP-25 (Sigma #S9684) which recognizes uncleaved SNAP-25 as well as SNAP-25 cleaved by the BoNT/A endopeptidase. The secondary antibody used was an HRP-conjugated anti-rabbit IgG (Sigma #A6154). Bands were detected by enhanced chemiluminescence and imaged using a pXi6 Access (Synoptics, UK). The intensity of bands was determined using GeneTools software (Syngene, Cambridge, UK) and the percentage of SNAP-25 cleaved at each concentration of BoNT calculated. Data were fitted to a 4-parameter logistic equation and pEC₅₀ calculated using GraphPad Prism version 6 (GraphPad).

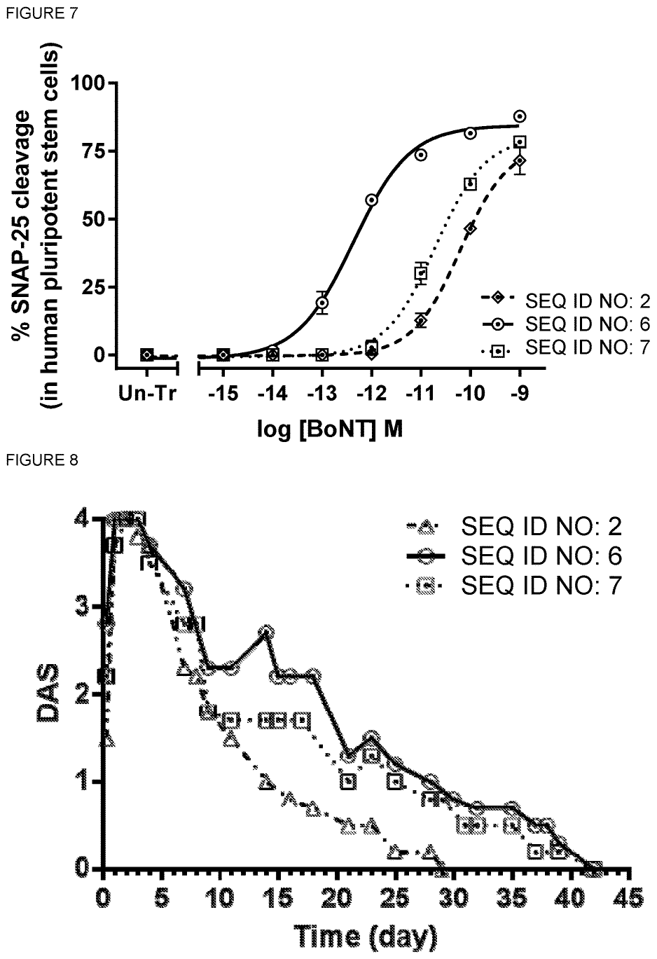

FIG. 7 shows that chimera 3B and 3C displayed greater potency than rBoNT/A1 in cleaving SNAP-25 in induced human pluripotent stem cells but the former significantly more so. This can be explained by the double mutation which increases the affinity of chimera 3B for the human synaptotagmin II protein receptor present in these cells (FIG. 7, Table 4).

TABLE 4
pEC₅₀ values.

| | pEC₅₀ ± SEM |
|---|---|
| rBoNT/A1 | 10.21 ± 0.05 |
| Chimera 3B | 12.38 ± 0.06 |
| Chimera 3C | 10.72 ± 0.08 |

### Digit Abduction Scoring (DAS) Assay—Safety Ratio

The method to measure the activity of BoNTs in the DAS assay is based on the startled response toe spreading reflex of mice, when suspended briefly by the tail. This reflex is scored as Digit Abduction Score (DAS) and is inhibited after administration of BoNT into the gastrocnemius-soleus muscles of the hind paw. Mice are suspended briefly by the tail to elicit a characteristic startled response in which the animal extends its hind limb and abducts its hind digits. (Aoki et al. 1999, Eur. J. Neurol.; 6 (suppl. 4) S3-S10).

On the day of injection, mice were anaesthetized in an induction chamber receiving isoflurane 3% in oxygen. Each mouse received an intramuscular injection of BoNT or vehicle (phosphate buffer containing 0.2% gelatine) in the gastrocnemius-soleus muscles of the right hind paw.

Following neurotoxin injection, the varying degrees of digit abduction were scored on a scale from zero to four, where 0=normal and 4=maximal reduction in digit abduction and leg extension. ED50 was determined by nonlinear adjustment analysis using average of maximal effect at each dose. The mathematical model used was the 4 parameters logistic model.

DAS was performed every 2 hours during the first day after dosing; thereafter it was performed 3 times a day for 4 days for all doses. Animals of the groups injected with vehicle and the lowest dose that induced during the first four days of injection a DAS of 4 were thereafter monitored until complete recovery of the muscle weakness to a DAS of 0 (no observed muscle weakness).

For calculation of the safety ratio all animals were weighed the day before toxin injection (DO) and thereafter once daily throughout the duration of the study. The average body weight, its standard deviation, and the standard error mean were calculated daily for each dose-group. To obtain the safety ratio for a BoNT (−10% ΔBW/ED₅₀), the dose at which at any time during the study the average weight of a dose-group was lower than 10% of the average weight at DO of that same dose-group was divided by the ED₅₀ for the BoNT studied. The lethal dose was defined as the dose at which one or more of the animals within that dose-group died.

FIG. 8 shows the duration of muscle weakening over time in the mouse digit abduction scoring assay for unmodified BoNT/A, chimera 3B and chimera 3C (SEQ ID NO: 2, 6 and 7 converted into a di-chain form), showing that the chimera has longer duration of action.

Table 5 below provides the ED₅₀ and DAS 4 doses determined for rBoNT/A1 and chimeras 3B and 3C in the mouse DAS assay. The table also provide the total duration of action for the DAS 4 dose until complete recovery of the muscle weakness to a DAS of 0 (no observed muscle weakness). In addition, the table shows the mouse lethal dose and the safety ratio (−10% ΔBW/ED₅₀), as defined in the text above. In comparison to rBoNT/A1, chimeras 3B and 3C have longer duration of action, a better safety ratio, and a higher lethal dose. Studies shown in FIG. 8 and Table 5 were performed in mice obtained from Janvier laboratories.

TABLE 5
DAS and Safety Ratios of the BoNT/AB chimeras.

| | ED₅₀ | | Total | | |
|---|---|---|---|---|---|
| | (DAS 2) | DAS 4 | duration of | Mouse | Safety |
| | Dose | dose | action (day) | lethal | ratio |
| | (pg/ | (pg/ | with lowest | dose | (−10% |
| | mouse) | mouse) | DAS 4 dose | (pg) | ΔBW/ED₅₀) |
| rBoNT/A1 | 0.9 | 2.3 | 29 | 18 | 4.5 |
| Chimera | 8.0 | 89 | 42 | 200 | 14.1 |
| 3B | | | | | |
| Chimera | 5.0 | 26 | 42 | 8.9 | 7.4 |
| 3C | | | | | |

### Example 4

Further Characterisation of a BoNT/AB Chimera (SEQ ID NO: 6 Converted into a Di-Chain Form)

BoNT/AB chimera SEQ ID NO: 6 converted into a di-chain form was tested in a mouse LD₅₀ assay yielding a result of 1.202 ng/kg. 1 Unit of SEQ ID NO: 6 therefore corresponds to 24.04 pg in this assay.

Additionally, said BoNT/AB chimera was tested in a rat DAS assay to determine the duration of action when compared to Dysport®. Results are presented in Table 6 below:

TABLE 6
Duration of action.

| | Dysport ® | BoNT/AB |
|---|---|---|
| | 3 U/rat | 300 pg/rat |
| | 15 U/kg | 1.5 ng/kg |
| Duration of Action (median days) | 21.9 | 47.7 |

In conclusion, the duration of action of BoNT/AB was much higher than Dysport®.

### Example 5

Calculation of a Unit Dose of Modified BoNT/a (SEQ ID NO: 6 Converted into a Di-Chain Form) for Treating Limb Spasticity

In view of pre-clinical pharmacology data, a suitable unit dose range (UD) for administration of modified BoNT/A in humans has been calculated.

A DAS ED₅₀ of 13 μg/kg was calculated for SEQ ID NO: 6. ED₅₀ is considered as a minimal pharmacologically active dose, which is approximately 300-fold lower than the no observed adverse effect level (NOAEL) of 4 ng/kg in the same animal species. An ED₅₀ of 13 μg/kg of SEQ ID NO: 6 in rats corresponds to a 0.8 ng dose for a human of 60 kg body weight.

Thus, the lower limit of a unit dose of 1000 μg was selected. An upper limit of the unit dose of 16,000 pg was selected, which is lower than the NOAEL of 4 ng/kg from both nonclinical safety species (rat and monkey) converted into human dose for 60 kg body weight.

In view of the improved safety profile the maximum total dose for the treatment of limb spasticity was set at 240,000 pg, which is derived from the NOAEL of 4 ng/kg from both nonclinical safety species (rat and monkey) converted into human dose for 60 kg body weight.

Advantageously, modified BoNT/A (SEQ ID NO: 6) can be injected to a greater number of muscles in the treatment of limb spasticity before reaching the maximum dose. This is a significant and advantageous finding leading to improved treatment of limb spasticity while providing clinicians with a greater range of treatment options. For the first time, it also provides the option of being able to treat additional large muscles such as those of the shoulder, while also treating the elbow, forearm, and/or wrist well within the maximum dose.

### Example 6

Dosage Regimen for Treating Adult Upper Limb Spasticity Using a Modified BoNT/a (SEQ ID NO: 6 Converted into a Di-Chain Form)

Modified BoNT/A is provided as a lyophilised powder in a vial containing 36 ng of modified BoNT/A per vial. The lyophilised powder is reconstituted. The unit dose (UD) is 1000-16,000 pg (42-666 Units [measured by mouse LD₅₀]).

Adult upper limb spasticity is treated by intramuscular injection according to the following dosage regimen (Table 7):

TABLE 7
Dosage regimen.

| Clinical | | | Total | |
|---|---|---|---|---|
| Patterns | Muscles Injected | Dosage | Volume | |
| Clenched fist | Flexor Digitorum Superficialis | 1x UD | 1 | mL |
| | (FDS) | | | |
| | Flexor Digitorum Profundus | 1x UD | 1 | mL |
| | (FDP) | | | |
| Flexed wrist | Flexor Carpi Radialis (FCR) | 1 x UD | 1 | mL |
| | Flexor Carpi Ulnaris (FCU) | 1 x UD | 1 | mL |
| Flexed elbow | Brachioradialis | 1 x UD | 1 | mL |
| | Brachialis | 2 x UD | 2 | mL |
| | Pronator Teres | 1 x UD | 1 | mL |
| | Biceps Brachii | 2 x UD | 2 | mL |
| | | 1 x or 2 x UD | 1-2 | mL |
| Adducted/ | Triceps Brachii (long head) | 2 x UD | 2 | mL |
| rotated | Pectoralis Major | 2 x UD | 2 | mL |
| shoulder | Subscapularis | 2 x UD | 2 | mL |
| | Latissimus Dorsi | 2 x UD | 2 | mL |

A maximum total dosage administered is 15×UD. This corresponds to 240,000 pg/9,990 Units. Thus, the clinician is able to tailor treatment to the patient with the knowledge that 15×UD can be administered without any concern of toxicity, thereby allowing the treatment of additional muscles of the subject, including the shoulder, and/or ensuring each muscle receives a pharmaceutically effective dose.

### Example 7

Dosage Regimen for Treating Adult Lower Limb Spasticity Using a Modified BoNT/a (SEQ ID NO: 6 Converted into a Di-Chain Form)

Modified BoNT/A is provided as a lyophilised powder in a vial containing 36 ng of modified BoNT/A per vial. The lyophilised powder is reconstituted.

The unit dose (UD) is 1000-16,000 pg (42-666 Units).

Adult lower limb spasticity is treated by intramuscular injection according to the following dosage regimen (Table 8):

TABLE 8
Dosage regimen.

| Muscles Injected | Dosage |
|---|---|
| Gastrocnemius (Medial head) | 1x UD |
| Gastrocnemius (Lateral head) | 1x UD |
| Soleus | 3x UD |
| Tibialis posterior | 2x UD |
| Flexor digitorum longus | 1x UD |
| Flexor hallucis longus | 1x or 2x UD |

A maximum total dosage administered is 15×UD. This corresponds to 240,000 pg/9,990 Units. Thus, the clinician is able to tailor treatment to the patient with the knowledge that 15×UD can be administered without any concern of toxicity, thereby allowing the treatment of additional muscles of the subject and/or ensuring each muscle receives a pharmaceutically effective dose.

### Example 8

Safety & Efficacy of Modified BoNT/a (SEQ ID NO: 6 Converted into a Di-Chain Form) in Humans

An integrated phase I/II multicentre, double-blinded, randomised, Dysport and placebo controlled, dose-escalation and dose-finding study to evaluate the safety and efficacy of a modified BoNT/A (SEQ ID NO: 6 converted into a di-chain form) in the treatment of adult upper limb spasticity was carried out for subjects 18 to 70 years of age, with spastic hemiparesis following stroke or traumatic brain injury (TBI).

The study included a human sequential dose escalation in cohorts of unique patients with adult upper limb spasticity with clenched fist pattern. All participants were injected in the flexor digitorum profundus (FDP) and flexor digitorum superficialis (FDS). Each muscle was injected with a single unit dose of modified BoNT/A, Dysport, or placebo. 5 cohorts were administered different (increasing) amounts of modified BoNT/A (SEQ ID NO: 6 converted into a di-chain form). Cohort 1 was administered 2×1,000 pg unit doses of modified BoNT/A (i.e. 2,000 pg maximum), while cohort 5 was administered 2×16,000 pg unit doses of modified BoNT/A (i.e. 32,000 pg maximum).

Results showed that all unit doses of modified BoNT/A tested, (i.e. up to 16,000 pg unit doses), were effective at muscle paralysis, safely tolerated, and no adverse effects were observed, despite the exceptionally high dosage per muscle. This shows that the modified BoNT/A does not diffuse away from the injection site and highlights the exceptional safety profile of modified BoNT/A (SEQ ID NO: 6 converted into a di-chain form). Based on these findings, it is considered credible that much higher unit doses can be administered per muscle without resultant adverse effects. Furthermore, given the lack of systemic diffusion of the toxin, it is credible that up to 15× the higher unit doses can be administered without safety concerns.

Thus, unit doses of 30,000 pg and 36,000 pg (each to be administered up to 15×) have been selected for therapeutic treatment of adult limb spasticity. The total doses administered during a treatment session will, therefore, be 450,000 pg and 540,000 pg, respectively.

### Example 9

Dosage Regimen for Treating Adult Upper Limb Spasticity Using a Modified BoNT/a (SEQ ID NO: 6 Converted into a Di-Chain Form)

Modified BoNT/A is provided as a lyophilised powder in vials each containing 36 ng of modified BoNT/A. The lyophilised powder is reconstituted.

The unit dose (UD) is 30,000 pg (1,248 Units [measured by mouse LD₅₀]) or 36,000 pg (1,498 Units [measured by mouse LD₅₀]).

Adult upper limb spasticity is treated by intramuscular injection according to the following dosage regimen (Table 9):

TABLE 9
Dosage regimen.

| Clinical | | | Total | |
|---|---|---|---|---|
| Patterns | Muscles Injected | Dosage | Volume | |
| Clenched fist | Flexor Digitorum Superficialis | 1x UD | 1 | mL |
| | (FDS) | | | |
| | Flexor Digitorum Profundus | 1x UD | 1 | mL |
| | (FDP) | | | |
| Flexed wrist | Flexor Carpi Radialis (FCR) | 1 x UD | 1 | mL |
| | Flexor Carpi Ulnaris (FCU) | 1 x UD | 1 | mL |
| Flexed elbow | Brachioradialis | 1 x UD | 1 | mL |
| | Brachialis | 2 x UD | 2 | mL |
| | Pronator Teres | 1 x UD | 1 | mL |
| | Biceps Brachii | 2 x UD | 2 | mL |
| | | 1 x or 2 x UD | 1-2 | mL |
| Adducted/ | Triceps Brachii (long head) | 2 x UD | 2 | mL |
| rotated | Pectoralis Major | 2 x UD | 2 | mL |
| shoulder | Subscapularis | 2 x UD | 2 | mL |
| | Latissimus Dorsi | 2 x UD | 2 | mL |

A maximum total dosage administered is 15×UD. This corresponds to 450,000 pg/18,719 Units or 540,000 pg/22,463 Units. Thus, the clinician is able to tailor treatment to the patient with the knowledge that 15×UD can be administered without any concern of toxicity, thereby allowing the treatment of additional muscles of the subject, including the shoulder, and/or ensuring each muscle receives a pharmaceutically effective dose.

### Example 10

Dosage Regimen for Treating Adult Lower Limb Spasticity Using a Modified BoNT/a (SEQ ID NO: 6 Converted into a Di-Chain Form)

Modified BoNT/A is provided as a lyophilised powder in vials each containing 36 ng of modified BoNT/A. The lyophilised powder is reconstituted.

The unit dose (UD) is 30,000 pg (1,248 Units [measured by mouse LD₅₀]) or 36,000 pg (1,498 Units [measured by mouse LD₅₀]).

Adult lower limb spasticity is treated by intramuscular injection according to the following dosage regimen (Table 10):

TABLE 10
Dosage regimen.

| Muscles Injected | Dosage |
|---|---|
| Gastrocnemius (Medial head) | 1x UD |
| Gastrocnemius (Lateral head) | 1x UD |
| Soleus | 3x UD |
| Tibialis posterior | 2x UD |
| Flexor digitorum longus | 1x UD |
| Flexor hallucis longus | 1x or 2x UD |

A maximum total dosage administered is 15×UD. This corresponds to 450,000 pg/18,719 Units or 540,000 pg/22,463 Units. Thus, the clinician is able to tailor treatment to the patient with the knowledge that 15×UD can be administered without any concern of toxicity, thereby allowing the treatment of additional muscles of the subject and/or ensuring each muscle receives a pharmaceutically effective dose.

### Example 11

Treatment of a Patient with Adult Upper Limb Spasticity

Bill, aged 53, is diagnosed by his GP with adult upper limb spasticity. Modified BoNT/A (SEQ ID NO: 6 converted into a di-chain form) is administered by way of one or more unit doses (1× unit dose=36,000 pg) to the following muscles as indicated below:

| Clinical | | | Total |
|---|---|---|---|
| Patterns | Muscles Injected | Dosage | Volume |
| Flexed wrist | Flexor Carpi Radialis (FCR) | 1 x UD | 1 mL |
| | Flexor Carpi Ulnaris (FCU) | 1 x UD | 1 mL |
| Flexed elbow | Brachioradialis | 1 x UD | 1 mL |
| | Brachialis | 2 x UD | 2 mL |
| | Pronator Teres | 1 x UD | 1 mL |
| | Biceps Brachii | 1 x UD | 1 mL |
| Adducted/rotated | Triceps Brachii (long head) | 2 x UD | 2 mL |
| shoulder | Pectoralis Major | 2 x UD | 2 mL |
| | Subscapularis | 2 x UD | 2 mL |
| | Latissimus Dorsi | 2 x UD | 2 mL |

The total dose administered is 540,000 pg modified BoNT/A (SEQ ID NO: 6 converted into a di-chain form).

The adult upper limb spasticity is alleviated and, owing to the long duration of the modified BoNT/A, Bill does not require further treatment for greater than 9 months. Thus, Bill receives less frequent injections (e.g. per year) when compared to an equivalent subject administered an unmodified BoNT/A. Additionally, Bill does not exhibit any side-effects owing to the improved safety profile of the modified BoNT/A.

### Example 12

Safety & Efficacy of Modified BoNT/a (SEQ ID NO: 6 Converted into a Di-Chain Form) in Humans

An integrated phase I/II multicentre, double-blinded, randomised, Dysport and placebo controlled, dose-escalation and dose-finding study to evaluate the safety and efficacy of a modified BoNT/A (SEQ ID NO: 6 [converted into a di-chain form]) in the treatment of adult upper limb spasticity was carried out for subjects 18 to 70 years of age, with spastic hemiparesis following stroke or traumatic brain injury (TBI).

The study included a human sequential dose escalation in cohorts of unique patients with adult upper limb spasticity with clenched fist pattern. All participants were injected in the flexor digitorum profundus (FDP) and flexor digitorum superficialis (FDS). Each muscle was injected with a single unit dose of modified BoNT/A, Dysport, or placebo. Subjects were administered modified BoNT/A (SEQ ID NO: 6 converted into a di-chain form) as follows, via: a 15,000 pg unit dose to the FDP and a 15,000 pg unit dose to the FDS (i.e. a 30,000 pg total dose), a 25,000 pg unit dose to the FDP and a 25,000 pg unit dose to the FDS (i.e. a 50,000 pg total dose), or a 36,000 pg unit dose to the FDP and a 36,000 pg unit dose to the FDS (i.e. a 72,000 pg total dose).

Results showed that all unit doses of modified BoNT/A tested were effective at muscle paralysis, safely tolerated, and no adverse effects were observed, despite the exceptionally high dosage per muscle (e.g. for the 25,000 pg and 36,000 pg unit dose). This shows that the modified BoNT/A does not diffuse away from the injection site and highlights the exceptional safety profile of modified BoNT/A (SEQ ID NO: 6 converted into a di-chain form).

Given the lack of systemic diffusion of the toxin, it is credible that up to 15× the higher unit doses can be administered without safety concerns. Thus, unit doses of 25,000 pg and 36,000 pg (each to be administered up to 15×) have been selected for therapeutic treatment of adult limb spasticity. The total doses administered during a treatment session will, therefore, be 375,000 pg and 540,000 pg, respectively.

### Example 13

Treatment of a Patient with Adult Lower Limb Spasticity

Ryan, aged 63, is diagnosed by his GP with adult lower limb spasticity. Modified BoNT/A (SEQ ID NO: 6 [converted into a di-chain form]) is administered by way of one or more unit doses (1× unit dose=25,000 pg) to a plurality of the following muscles indicated below:

| Muscles Injected | Dosage |
|---|---|
| Medial gastrocnemius | 1 x UD |
| Lateral gastrocnemius | 1 x UD |
| Tibialis anterior | 1 x UD |
| Flexor digitorum longus | 1 x UD |
| Flexor digitorum brevis | 1 x UD |
| Flexor hallucis longus | 1 x UD |
| Flexor hallucis brevis | 1 x UD |
| Gracilis | 1 x UD |
| Gluteus maximus | 1 x UD |
| Soleus | 3 x UD |
| Tibialis posterior | 2 x UD |
| Rectus Femoris | 2 x UD |
| A hamstring muscle (e.g. the | 2 x UD |
| biceps femoris, the | |
| semimembranosus, and/or | |
| the semitendinosus) | |
| Adductor magnus | 2 x UD |

The maximum dosage is 15×UD (i.e. 375,000 pg) of the modified BoNT/A.

The spasticity is alleviated and, owing to the long duration of the modified BoNT/A, Ryan does not require further treatment for greater than 8 months. Thus, Ryan receives less frequent injections (e.g. per year) when compared to an equivalent subject administered an unmodified BoNT/A. Additionally, Ryan does not exhibit any side-effects owing to the improved safety profile of the modified BoNT/A.

### Example 14

Treatment of a Patient with Paediatric Lower Limb Spasticity

Sharon, aged 7, is diagnosed by her GP with paediatric lower limb spasticity. Modified BoNT/A (SEQ ID NO: 6 [converted into a di-chain form]) is administered by way of one or more unit doses (1× unit dose=12,500 pg) to a plurality of the following muscles indicated below:

| Muscles Injected | Dosage |
|---|---|
| Medial gastrocnemius | 1 x UD |
| Lateral gastrocnemius | 1 x UD |
| Tibialis anterior | 1 x UD |
| Flexor digitorum longus | 1 x UD |
| Flexor digitorum brevis | 1 x UD |
| Flexor hallucis longus | 1 x UD |
| Flexor hallucis brevis | 1 x UD |
| Gracilis | 1 x UD |
| Gluteus maximus | 1 x UD |
| Soleus | 3 x UD |
| Tibialis posterior | 2 x UD |
| Rectus Femoris | 2 x UD |
| A hamstring muscle (e.g. the | 2 x UD |
| biceps femoris, the | |
| semimembranosus, and/or | |
| the semitendinosus | |
| Adductor magnus | 2 x UD |

The maximum dosage is 15×UD (i.e. 187,500 pg) of the modified BoNT/A.

The spasticity is alleviated and, owing to the long duration of the modified BoNT/A, Sharon does not require further treatment for greater than 8 months. Thus, Sharon receives less frequent injections (e.g. per year) when compared to an equivalent subject administered an unmodified BoNT/A. Additionally, Sharon does not exhibit any side-effects owing to the improved safety profile of the modified BoNT/A.

### Example 15

Treatment of a Patient with Paediatric Lower Limb Spasticity

Timmy, aged 8, is diagnosed by his GP with paediatric lower limb spasticity. Modified BoNT/A (SEQ ID NO: 6 [converted into a di-chain form]) is administered by way of one or more unit doses (1× unit dose=18,000 pg) to a plurality of the following muscles indicated below:

| Muscles Injected | Dosage |
|---|---|
| Medial gastrocnemius | 1 x UD |
| Lateral gastrocnemius | 1 x UD |
| Tibialis anterior | 1 x UD |
| Flexor digitorum longus | 1 x UD |
| Flexor digitorum brevis | 1 x UD |
| Flexor hallucis longus | 1 x UD |
| Flexor hallucis brevis | 1 x UD |
| Gracilis | 1 x UD |
| Gluteus maximus | 1 x UD |
| Soleus | 3 x UD |
| Tibialis posterior | 2 x UD |
| Rectus Femoris | 2 x UD |
| A hamstring muscle (e.g. the | 2 x UD |
| biceps femoris, the | |
| semimembranosus, and/or | |
| the semitendinosus) | |
| Adductor magnus | 2 x UD |

The maximum dosage is 15×UD (i.e. 270,000 pg) of the modified BoNT/A.

The spasticity is alleviated and, owing to the long duration of the modified BoNT/A, Timmy does not require further treatment for greater than 9 months. Thus, Timmy receives less frequent injections (e.g. per year) when compared to an equivalent subject administered an unmodified BoNT/A. Additionally, Timmy does not exhibit any side-effects owing to the improved safety profile of the modified BoNT/A.

### Example 16

Treatment of a Patient with Adult Upper Limb Spasticity

Larry, aged 21, is diagnosed by his GP with upper limb spasticity. Modified BoNT/A (SEQ ID NO: 6 [converted into a di-chain form]) is administered by way of one or more unit doses (1× unit dose=25,000 pg) to a plurality of the following muscles indicated below:

| Muscles Injected | Dosage |
|---|---|
| Flexor Digitorum Superficialis (FDS) | 1x UD |
| Flexor Digitorum Profundus (FDP) | 1x UD |
| Flexor Carpi Radialis (FCR) | 1 x UD |
| Flexor Carpi Ulnaris (FCU) | 1 x UD |
| Brachioradialis | 1 x UD |
| Brachialis | 2 x UD |
| Biceps Brachii | 2 x UD |
| Triceps Brachii (long head) | 2 x UD |
| Pectoralis Major | 2 x UD |
| Subscapularis | 2 x UD |
| Latissimus Dorsi | 2 x UD |

The maximum dosage is 15×UD (i.e. 375,000 pg) of the modified BoNT/A.

The spasticity is alleviated and, owing to the long duration of the modified BoNT/A, Larry does not require further treatment for greater than 9 months. Thus, Larry receives less frequent injections (e.g. per year) when compared to an equivalent subject administered an unmodified BoNT/A. Additionally, Larry does not exhibit any side-effects owing to the improved safety profile of the modified BoNT/A.

### Example 17

Treatment of a Patient with Adult Upper Limb Spasticity

Loretta, aged 39, is diagnosed by her GP with upper limb spasticity. Modified BoNT/A (SEQ ID NO: 6 [converted into a di-chain form]) is administered by way of one or more unit doses (1× unit dose=36,000 pg) to a plurality of the following muscles indicated below:

| Muscles Injected | Dosage |
|---|---|
| Flexor Digitorum Superficialis (FDS) | 1x UD |
| Flexor Digitorum Profundus (FDP) | 1x UD |
| Flexor Carpi Radialis (FCR) | 1 x UD |
| Flexor Carpi Ulnaris (FCU) | 1 x UD |
| Brachioradialis | 1 x UD |
| Brachialis | 2 x UD |
| Biceps Brachii | 2 x UD |
| Triceps Brachii (long head) | 2 x UD |
| Pectoralis Major | 2 x UD |
| Subscapularis | 2 x UD |
| Latissimus Dorsi | 2 x UD |

The maximum dosage is 15×UD (i.e. 540,000 pg) of the modified BoNT/A.

The spasticity is alleviated and, owing to the long duration of the modified BoNT/A, Loretta does not require further treatment for greater than 9 months. Thus, Loretta receives less frequent injections (e.g. per year) when compared to an equivalent subject administered an unmodified BoNT/A. Additionally, Loretta does not exhibit any side-effects owing to the improved safety profile of the modified BoNT/A.

### Example 18

Treatment of a Patient with Paediatric Upper Limb Spasticity

Jack, aged 13, is diagnosed by his GP with upper limb spasticity. Modified BoNT/A (SEQ ID NO: 6 [converted into a di-chain form]) is administered by way of one or more unit doses (1× unit dose=12,500 pg) to a plurality of the following muscles indicated below:

| Muscles Injected | Dosage |
|---|---|
| Flexor Digitorum Superficialis (FDS) | 1x UD |
| Flexor Digitorum Profundus (FDP) | 1x UD |
| Flexor Carpi Radialis (FCR) | 1 x UD |
| Flexor Carpi Ulnaris (FCU) | 1 x UD |
| Brachioradialis | 1 x UD |
| Brachialis | 2 x UD |
| Biceps Brachii | 2 x UD |
| Triceps Brachii (long head) | 2 x UD |
| Pectoralis Major | 2 x UD |
| Subscapularis | 2 x UD |
| Latissimus Dorsi | 2 x UD |

The maximum dosage is 15×UD (i.e. 187,500 pg) of the modified BoNT/A.

The spasticity is alleviated and, owing to the long duration of the modified BoNT/A, Jack does not require further treatment for greater than 9 months. Thus, Jack receives less frequent injections (e.g. per year) when compared to an equivalent subject administered an unmodified BoNT/A. Additionally, Jack does not exhibit any side-effects owing to the improved safety profile of the modified BoNT/A.

### Example 19

Treatment of a Patient with Paediatric Upper Limb Spasticity

Julia, aged 11, is diagnosed by her GP with upper limb spasticity. Modified BoNT/A (SEQ ID NO: 6 [converted into a di-chain form]) is administered by way of one or more unit doses (1× unit dose=18,000 pg) to a plurality of the following muscles indicated below:

| Muscles Injected | Dosage |
|---|---|
| Flexor Digitorum Superficialis (FDS) | 1x UD |
| Flexor Digitorum Profundus (FDP) | 1x UD |
| Flexor Carpi Radialis (FCR) | 1 x UD |
| Flexor Carpi Ulnaris (FCU) | 1 x UD |
| Brachioradialis | 1 x UD |
| Brachialis | 2 x UD |
| Biceps Brachii | 2 x UD |
| Triceps Brachii (long head) | 2 x UD |
| Pectoralis Major | 2 x UD |
| Subscapularis | 2 x UD |
| Latissimus Dorsi | 2 x UD |

The maximum dosage is 15×UD (i.e. 270,000 pg) of the modified BoNT/A.

The spasticity is alleviated and, owing to the long duration of the modified BoNT/A, Julia does not require further treatment for greater than 9 months. Thus, Julia receives less frequent injections (e.g. per year) when compared to an equivalent subject administered an unmodified BoNT/A. Additionally, Julia does not exhibit any side-effects owing to the improved safety profile of the modified BoNT/A.

### Example 20

Treatment of a Patient with Adult Lower Limb Spasticity

Clare, aged 57, is diagnosed by her GP with adult lower limb spasticity. Modified BoNT/A (SEQ ID NO: 6 [converted into a di-chain form]) is administered by way of one or more unit doses (1× unit dose=36,000 pg) to a plurality of the following muscles indicated below:

| Muscles Injected | Dosage |
|---|---|
| Medial gastrocnemius | 1 x UD |
| Lateral gastrocnemius | 1 x UD |
| Tibialis anterior | 1 x UD |
| Flexor digitorum longus | 1 x UD |
| Flexor digitorum brevis | 1 x UD |
| Flexor hallucis longus | 1 x UD |
| Flexor hallucis brevis | 1 x UD |
| Gracilis | 1 x UD |
| Gluteus maximus | 1 x UD |
| Soleus | 3 x UD |
| Tibialis posterior | 2 x UD |
| Rectus Femoris | 2 x UD |
| A hamstring muscle (e.g. the | 2 x UD |
| biceps femoris, the | |
| semimembranosus, and/or | |
| the semitendinosus) | |
| Adductor magnus | 2 x UD |

The maximum dosage is 15×UD (i.e. 540,000 pg) of the modified BoNT/A.

The spasticity is alleviated and, owing to the long duration of the modified BoNT/A, Clare does not require further treatment for greater than 8 months. Thus, Clare receives less frequent injections (e.g. per year) when compared to an equivalent subject administered an unmodified BoNT/A. Additionally, Clare does not exhibit any side-effects owing to the improved safety profile of the modified BoNT/A.

### CLAUSES

1. A modified BoNT/A for use in treating limb spasticity (adult limb spasticity), wherein the modified BoNT/A is administered by intramuscular injection to a plurality of affected muscles of a subject,
   wherein the modified BoNT/A is administered by way of a unit dose of greater than 17,000 pg of modified BoNT/A at the plurality of affected muscles,
   wherein the plurality of affected muscles are selected from:
      a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head, the gastrocnemius lateral head, the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
      a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, and the gastrocnemius; and
   wherein a single unit dose is administered at an affected first group muscle and/or multiple unit doses are administered at an affected second group muscle, and wherein the total dose administered during the treatment is up to 600,000 pg, and
   wherein the modified BoNT/A comprises a BoNT/A light-chain and translocation domain, and a BoNT/B receptor binding domain (H_{C} domain).
2. The modified BoNT/A for use according to clause 1, wherein the limb spasticity is upper limb spasticity.
3. The modified BoNT/A for use according to clause 1 or clause 2, wherein the unit dose is greater than 17,000 pg up to 40,000 pg (e.g. 17,001 pg up to 40,000 pg) of modified BoNT/A.
4. The modified BoNT/A for use according to any one of clauses 1-3, wherein the unit dose is greater than 23,000 pg up to 38,000 pg, (e.g. 23,001 pg up to 38,000 pg) of modified BoNT/A.
5. The modified BoNT/A for use according to any one of clauses 1-4, wherein the unit dose is greater than 34,000 pg up to 38,000 pg (e.g. 34,001 pg up to 38,000 pg) of modified BoNT/A.
6. The modified BoNT/A for use according to any one of clauses 1-5, wherein the total dose administered is 250,000 pg to 600,000 pg of modified BoNT/A, (e.g. 255,000 pg to 600,000 pg).
7. The modified BoNT/A for use according to any one of clauses 1-6, wherein the total dose administered is greater than 255,000 pg to 600,000 pg of modified BoNT/A.
8. The modified BoNT/A for use according to any one of clauses 1-7, wherein the total dose administered is 350,000 to 600,000 pg of modified BoNT/A.
9. The modified BoNT/A for use according to any one of clauses 1-8, wherein the total dose administered is 500,000 to 600,000 pg of modified BoNT/A.
10. A modified BoNT/A for use in treating paediatric limb spasticity, wherein the modified BoNT/A is administered by intramuscular injection to a plurality of affected muscles of a subject,
   wherein the modified BoNT/A is administered by way of a unit dose of greater than 8,500 pg of modified BoNT/A at the plurality of affected muscles,
   wherein the plurality of affected muscles are selected from:
      a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head, the gastrocnemius lateral head, the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
      a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, and the gastrocnemius; and
   wherein a single unit dose is administered at an affected first group muscle and/or multiple unit doses are administered at an affected second group muscle, and wherein the total dose administered during the treatment is up to 300,000 pg, and
   wherein the modified BoNT/A comprises a BoNT/A light-chain and translocation domain, and a BoNT/B receptor binding domain (H_{C} domain).
11. The modified BoNT/A for use according to clause 10, wherein the paediatric limb spasticity is paediatric upper limb spasticity.
12. The modified BoNT/A for use according to clause 10 or clause 11, wherein the unit dose is greater than 8,500 pg up to 20,000 pg (e.g. 8,501 pg up to 20,000 pg) of modified BoNT/A, preferably wherein the unit dose is greater than 8,500 pg up to 18,000 pg.
13. The modified BoNT/A for use according to any one of clauses 10-12, wherein the unit dose is greater than 11,500 pg up to 19,000 pg, (e.g. 11,501 pg up to 19,000 pg) of modified BoNT/A.
14. The modified BoNT/A for use according to any one of clauses 10-13, wherein the unit dose is greater than 17,000 pg up to 19,000 pg (e.g. 17,001 pg up to 19,000 pg) of modified BoNT/A.
15. The modified BoNT/A for use according to any one of clauses 10-14, wherein the total dose administered is greater than 125,000 pg to 300,000 pg (e.g. 127,500 pg to 300,000 pg) of modified BoNT/A.
16. The modified BoNT/A for use according to any one of clauses 10-15, wherein the total dose administered is greater than 127,500 pg to 300,000 pg of modified BoNT/A.
17. The modified BoNT/A for use according to any one of clauses 10-16, wherein the total dose administered is 175,000 to 300,000 pg of modified BoNT/A.
18. The modified BoNT/A for use according to any one of clauses 10-17, wherein the total dose administered is 250,000 to 300,000 pg of modified BoNT/A.
19. A modified BoNT/A for use in treating limb spasticity (adult limb spasticity), wherein the modified BoNT/A is administered by intramuscular injection to a plurality of affected muscles of a subject,
   wherein the modified BoNT/A is administered by way of a unit dose of greater than 707 Units of modified BoNT/A at the plurality of affected muscles, and wherein 1 Unit is an amount of the modified BoNT/A that corresponds to the calculated median lethal dose (LD₅₀) in mice,
   wherein the plurality of affected muscles are selected from:
      a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head, the gastrocnemius lateral head, the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
      a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, and the gastrocnemius; and
   wherein a single unit dose is administered at an affected first group muscle and/or multiple unit doses are administered at an affected second group muscle, and wherein the total dose administered during the treatment is up to 24,958 Units, and
   wherein the modified BoNT/A comprises a BoNT/A light-chain and translocation domain, and a BoNT/B receptor binding domain (H_{C} domain).
20. The modified BoNT/A for use according to clause 19, wherein the limb spasticity is upper limb spasticity.
21. The modified BoNT/A for use according to clause 19 or clause 20, wherein the unit dose is greater than 707 Units up to 1,664 Units (e.g. 708 Units up to 1,664 Units) of modified BoNT/A.
22. The modified BoNT/A for use according to any one of clauses 19-21, wherein the unit dose is greater than 949 Units up to 1,581 Units, (e.g. 950 Units up to 1,581 Units) of modified BoNT/A.
23. The modified BoNT/A for use according to any one of clauses 19-22, wherein the unit dose is greater than 1,414 Units up to 1,581 Units (e.g. 1,415 Units up to 1,581 Units) of modified BoNT/A.
24. The modified BoNT/A for use according to any one of clauses 19-23, wherein the total dose administered is greater than 10,399 Units to 24,958 Units (e.g. 10,607 Units to 24,958 Units) of modified BoNT/A.
25. The modified BoNT/A for use according to any one of clauses 19-24, wherein the total dose administered is greater than 10,607 Units to 24,958 Units of modified BoNT/A.
26. The modified BoNT/A for use according to any one of clauses 19-25, wherein the total dose administered is 14,559 Units to 24,958 Units of modified BoNT/A.
27. The modified BoNT/A for use according to any one of clauses 19-26, wherein the total dose administered is 20,799 Units to 24,958 Units of modified BoNT/A.
28. A modified BoNT/A for use in treating paediatric limb spasticity, wherein the modified BoNT/A is administered by intramuscular injection to a plurality of affected muscles of a subject,
   wherein the modified BoNT/A is administered by way of a unit dose greater than 353.5 Units of modified BoNT/A at the plurality of affected muscles, and wherein 1 Unit is an amount of the modified BoNT/A that corresponds to the calculated median lethal dose (LD₅₀) in mice,
   wherein the plurality of affected muscles are selected from:
      a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head, the gastrocnemius lateral head, the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
      a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, and the gastrocnemius; and
   wherein a single unit dose is administered at an affected first group muscle and/or multiple unit doses are administered at an affected second group muscle, and wherein the total dose administered during the treatment is up to 12,479 Units, and
   wherein the modified BoNT/A comprises a BoNT/A light-chain and translocation domain, and a BoNT/B receptor binding domain (H_{C} domain).
29. The modified BoNT/A for use according to clause 28, wherein the paediatric limb spasticity is paediatric upper limb spasticity.
30. The modified BoNT/A for use according to clause 28 or clause 29, wherein the unit dose is greater than 353.5 Units up to 832 Units (e.g. 354 Units up to 832 Units) of modified BoNT/A, preferably wherein the unit dose is greater than 353.5 Units up to 749 Units (e.g. 354 Units up to 749 Units).
31. The modified BoNT/A for use according to any one of clauses 28-30, wherein the unit dose is greater than 478 Units up to 749 Units (e.g. 479 Units up to 749 Units) of modified BoNT/A.
32. The modified BoNT/A for use according to any one of clauses 28-31, wherein the unit dose is greater than 707 Units up to 749 Units (e.g. 708 Units up to 749 Units) of modified BoNT/A.
33. The modified BoNT/A for use according to any one of clauses 28-32, wherein the total dose administered is greater than 5,200 Units to 12,479 Units (e.g. 5,304 Units to 12,479 Units) of modified BoNT/A.
34. The modified BoNT/A for use according to any one of clauses 28-33, wherein the total dose administered is greater than 5,304 Units to 12,479 Units of modified BoNT/A.
35. The modified BoNT/A for use according to any one of clauses 28-34, wherein the total dose administered is 7,280 Units to 12,479 Units of modified BoNT/A.
36. The modified BoNT/A for use according to any one of clauses 28-35, wherein the total dose administered is 10,400 Units up to 12,479 Units of modified BoNT/A.
37. The modified BoNT/A for use according to any one of the preceding clauses, wherein the modified BoNT/A comprises a polypeptide sequence having at least 70% sequence identity to SEQ ID NO: 6.
38. The modified BoNT/A for use according to any one of the preceding clauses, wherein the modified BoNT/A comprises the polypeptide sequence of SEQ ID NO: 6.
39. The modified BoNT/A for use according to any one of the preceding clauses, wherein the modified BoNT/A consists of the polypeptide sequence of SEQ ID NO: 6.
40. The modified BoNT/A for use according to any one of the preceding clauses, wherein the modified BoNT/A has a Safety Ratio of greater than 7, wherein the Safety Ratio is calculated as: dose of toxin required for −10% bodyweight change measured as pg/mouse divided by DAS ED₅₀ measured as pg/mouse, wherein ED₅₀=dose required to produce a DAS score of 2.
41. The modified BoNT/A for use according to any one of the preceding clauses, wherein the plurality of muscles are selected from:
   a first group comprising: the flexor digitorum superficialis, the flexor digitorum profundus, the flexor carpi radialis, the flexor carpi ulnaris, the brachioradialis, the pronator teres, the deltoid, the levator scapulae, the pronator quadratus, the flexor policis longus, the adductor policis, the flexor policis brevis, the palmaris longus, the lumbricales, and the opponens policis; and
   a second group comprising: the triceps brachii (long head), the subscapularis, the pectoralis (e.g. the pectoralis major), the latissimus dorsi, the biceps brachii, the brachialis, the brachioradialis, and the teres major.
42. A unit dosage form of modified BoNT/A, the unit dosage form comprising:
   (a) greater than 707 Units of modified BoNT/A, wherein 1 Unit is an amount of the modified BoNT/A that corresponds to the calculated median lethal dose (LD₅₀) in mice; or
   (b) greater than 17,000 pg of modified BoNT/A; and
   (c) optionally a pharmaceutically acceptable carrier, excipient, adjuvant, and/or salt,
wherein the modified BoNT/A comprises a BoNT/A light-chain and translocation domain, and a BoNT/B receptor binding domain (H_{C} domain).
43. The unit dosage form according to clause 42, comprising:
   (a) greater than 707 Units up to 1,664 Units (e.g. 708 Units to 1,664 Units) of modified BoNT/A; or
   (b) greater than 17,000 pg up to 40,000 pg (e.g. 17,001 pg up to 40,000 pg) of modified BoNT/A.
44. A kit comprising:
   (a) the unit dosage form according to clause 42 or 43; and
   (b) instructions for use of the same in treating limb spasticity; and
   (c) optionally a diluent.
45. A unit dosage form of modified BoNT/A (e.g. for treating adult limb spasticity), the unit dosage form comprising:
   (a) greater than 353.5 Units of modified BoNT/A, wherein 1 Unit is an amount of the modified BoNT/A that corresponds to the calculated median lethal dose (LD₅₀) in mice; or
   (b) greater than 8,500 pg of modified BoNT/A; and
   (c) optionally a pharmaceutically acceptable carrier, excipient, adjuvant, and/or salt,
wherein the modified BoNT/A comprises a BoNT/A light-chain and translocation domain, and a BoNT/B receptor binding domain (H_{C} domain).
46. The unit dosage form according to clause 45, comprising:
   (a) greater than 353.5 Units up to 832 Units (e.g. 354 Units up to 832 Units) of modified BoNT/A; or
   (b) greater than 8,500 pg up to 20,000 pg (e.g. 8,501 pg up to 20,000 pg) of modified BoNT/A.
47. A kit comprising:
   (a) the unit dosage form according to clause 45 or 46; and
   (b) instructions for use of the same in treating paediatric limb spasticity; and
   (c) optionally a diluent.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1. A modified botulinum neurotoxin A (BoNT/A) for use in treating limb spasticity, wherein the modified BoNT/A is administered by intramuscular injection to a plurality of affected muscles of a subject,
wherein the modified BoNT/A is administered by way of a unit dose of greater than 17,000 pg of modified BoNT/A at the plurality of affected muscles,
wherein the plurality of affected muscles are selected from:
a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius; and
wherein a single unit dose is administered at an affected first group muscle and/or multiple unit doses are administered at an affected second group muscle, and wherein the total dose administered during the treatment is up to 600,000 pg, and
wherein the modified BoNT/A comprises a BoNT/A light-chain and translocation domain, and a BoNT/B receptor binding domain (H_{C} domain).

2. A method for treating limb spasticity, the method comprising administering a modified botulinum neurotoxin A (BoNT/A) by intramuscular injection to a plurality of affected muscles of a subject,
wherein the modified BoNT/A is administered by way of a unit dose of greater than 17,000 pg of modified BoNT/A at the plurality of affected muscles,
wherein the plurality of affected muscles are selected from:
a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius; and
wherein a single unit dose is administered at an affected first group muscle and/or multiple unit doses are administered at an affected second group muscle, and wherein the total dose administered during the treatment is up to 600,000 pg, and
wherein the modified BoNT/A comprises a BoNT/A light-chain and translocation domain, and a BoNT/B receptor binding domain (H_{C} domain).

3. Use of a modified botulinum neurotoxin A (BoNT/A) in the manufacture of a medicament for treating limb spasticity, wherein the modified BoNT/A is administered by intramuscular injection to a plurality of affected muscles of a subject,
wherein the modified BoNT/A is administered by way of a unit dose of greater than 17,000 pg of modified BoNT/A at the plurality of affected muscles,
wherein the plurality of affected muscles are selected from:
a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius; and
wherein a single unit dose is administered at an affected first group muscle and/or multiple unit doses are administered at an affected second group muscle, and wherein the total dose administered during the treatment is up to 600,000 pg, and
wherein the modified BoNT/A comprises a BoNT/A light-chain and translocation domain, and a BoNT/B receptor binding domain (H_{C} domain).

4. The modified BoNT/A for use, method or use according to any one of the preceding claims, wherein the unit dose is greater than 17,000 pg up to 40,000 pg (e.g. 17,001 pg up to 40,000 pg) of modified BoNT/A.

5. The modified BoNT/A for use, method or use according to any one of the preceding claims, wherein the unit dose is 20,000 pg to 30,000 pg, preferably 24,000 pg to 26,000 pg, more preferably 25,000 pg, of modified BoNT/A.

6. The modified BoNT/A for use, method or use according to any one of claims 1-4, wherein the unit dose is 30,000 pg to 40,000 pg, preferably 35,000 pg to 37,000 pg, more preferably 36,000 pg, of modified BoNT/A.

7. The modified BoNT/A for use, method or use according to any one of claims 1-4, wherein the unit dose is greater than 23,000 pg up to 38,000 pg, (e.g. 23,001 pg up to 38,000 pg) of modified BoNT/A.

8. The modified BoNT/A for use, method or use according to any one of claims 1-4 or 6-7, wherein the unit dose is greater than 34,000 pg up to 38,000 pg (e.g. 34,001 pg up to 38,000 pg) of modified BoNT/A.

9. The modified BoNT/A for use, method or use according to any one of the preceding claims, wherein the total dose administered is 250,000 pg to 600,000 pg (e.g. 255,000 pg to 600,000 pg) of modified BoNT/A.

10. The modified BoNT/A for use, method or use according to any one of the preceding claims, wherein the total dose administered is greater than 255,000 pg to 600,000 pg of modified BoNT/A.

11. The modified BoNT/A for use, method or use according to any one of the preceding claims, wherein the total dose administered is 350,000 to 600,000 pg of modified BoNT/A.

12. The modified BoNT/A for use, method or use according to any one of claims 1-10, wherein the total dose administered is up to 300,000 pg to 450,000 pg, preferably up to 360,000 pg to 390,000 pg, more preferably up to 375,000 pg, of modified BoNT/A.

13. The modified BoNT/A for use, method or use according to any one of claims 1-11, wherein the total dose administered is up to 450,000 pg to 600,000 pg, preferably up to 525,000 pg to 555,000 pg, more preferably up to 540,000 pg, of modified BoNT/A.

14. The modified BoNT/A for use, method or use according to any one of claims 1-11 or 13, wherein the total dose administered is 500,000 to 600,000 pg of modified BoNT/A.

15. A modified botulinum neurotoxin A (BoNT/A) for use in treating limb spasticity, wherein the modified BoNT/A is administered by intramuscular injection to a plurality of affected muscles of a subject,
wherein the modified BoNT/A is administered by way of a unit dose of greater than 707 Units of modified BoNT/A at the plurality of affected muscles, and wherein 1 Unit is an amount of the modified BoNT/A that corresponds to the calculated median lethal dose (LD₅₀) in mice,
wherein the plurality of affected muscles are selected from:
a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius; and
wherein a single unit dose is administered at an affected first group muscle and/or multiple unit doses are administered at an affected second group muscle, and wherein the total dose administered during the treatment is up to 24,958 Units, and
wherein the modified BoNT/A comprises a BoNT/A light-chain and translocation domain, and a BoNT/B receptor binding domain (H_{C} domain).

16. A method for treating limb spasticity, the method comprising administering a modified botulinum neurotoxin A (BoNT/A) by intramuscular injection to a plurality of affected muscles of a subject,
wherein the modified BoNT/A is administered by way of a unit dose of greater than 707 Units of modified BoNT/A at the plurality of affected muscles, and wherein 1 Unit is an amount of the modified BoNT/A that corresponds to the calculated median lethal dose (LD₅₀) in mice,
wherein the plurality of affected muscles are selected from:
a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius; and
wherein a single unit dose is administered at an affected first group muscle and/or multiple unit doses are administered at an affected second group muscle, and wherein the total dose administered during the treatment is up to 24,958 Units, and
wherein the modified BoNT/A comprises a BoNT/A light-chain and translocation domain, and a BoNT/B receptor binding domain (H_{C} domain).

17. Use of a modified botulinum neurotoxin A (BoNT/A) in the manufacture of a medicament for treating limb spasticity, wherein the modified BoNT/A is administered by intramuscular injection to a plurality of affected muscles of a subject,
wherein the modified BoNT/A is administered by way of a unit dose of greater than 707 Units of modified BoNT/A at the plurality of affected muscles, and wherein 1 Unit is an amount of the modified BoNT/A that corresponds to the calculated median lethal dose (LD₅₀) in mice,
wherein the plurality of affected muscles are selected from:
a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius; and
wherein a single unit dose is administered at an affected first group muscle and/or multiple unit doses are administered at an affected second group muscle, and wherein the total dose administered during the treatment is up to 24,958 Units, and
wherein the modified BoNT/A comprises a BoNT/A light-chain and translocation domain, and a BoNT/B receptor binding domain (H_{C} domain).

18. The modified BoNT/A for use, method or use according to any one of claims 15-17, wherein the unit dose is greater than 707 Units up to 1,664 Units (e.g. 708 Units up to 1,664 Units) of modified BoNT/A.

19. The modified BoNT/A for use, method or use according to any one of claims 15-18, wherein the unit dose is 832 Units to 1,248 Units, preferably 998 Units to 1,082 Units, more preferably 1,040 Units, of modified BoNT/A.

20. The modified BoNT/A for use, method or use according to any one of claims 15-18, wherein the unit dose is 1,248 Units to 1,664 Units, preferably 1,456 Units to 1,539 Units, more preferably 1,498 Units, of modified BoNT/A.

21. The modified BoNT/A for use, method or use according to any one of claims 15-18, wherein the unit dose is greater than 949 Units up to 1,581 Units or greater than 957 Units up to 1,581 Units, (e.g. 950 Units up to 1,581 Units or 958 Units up to 1,581 Units) of modified BoNT/A.

22. The modified BoNT/A for use, method or use according to any one of claims 15-18 or 20-21, wherein the unit dose is greater than 1,414 Units up to 1,581 Units (e.g. 1,415 Units up to 1,581 Units) of modified BoNT/A.

23. The modified BoNT/A for use, method or use according to any one of claims 15-22, wherein the total dose administered is 10,399 Units to 24,958 Units (e.g. 10,607 Units to 24,958 Units) of modified BoNT/A.

24. The modified BoNT/A for use, method or use according to any one of claims 15-23, wherein the total dose administered is greater than 10,607 Units to 24,958 Units of modified BoNT/A.

25. The modified BoNT/A for use, method or use according to any one of claims 15-24, wherein the total dose administered is up to 12,479 Units to 18,719 Units, preferably up to 14,975 Units to 16,223 Units, more preferably up to 15,599 Units, of modified BoNT/A.

26. The modified BoNT/A for use, method or use according to any one of claims 12-20, wherein the total dose administered is 14,559 Units to 24,958 Units of modified BoNT/A.

27. The modified BoNT/A for use, method or use according to any one of claims 15-24 or 26, wherein the total dose administered is up to 18,719 Units to 24,958 Units, preferably up to 21,839 Units to 23,087 Units, more preferably up to 22,463 Units, of modified BoNT/A.

28. The modified BoNT/A for use, method or use according to any one of claims 15-24 or 26-27, wherein the total dose administered is 20,799 Units to 24,958 Units of modified BoNT/A.

29. The modified BoNT/A for use, method or use according to any one of the preceding claims, wherein the spasticity is adult limb spasticity and the subject is an adult subject.

30. The modified BoNT/A for use, method or use according to any one of the preceding claims, wherein the limb spasticity is upper limb spasticity.

31. A modified botulinum neurotoxin A (BoNT/A) for use in treating paediatric limb spasticity, wherein the modified BoNT/A is administered by intramuscular injection to a plurality of affected muscles of a subject,
wherein the modified BoNT/A is administered by way of a unit dose of greater than 8,500 pg of modified BoNT/A at the plurality of affected muscles,
wherein the plurality of affected muscles are selected from:
a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius; and
wherein a single unit dose is administered at an affected first group muscle and/or multiple unit doses are administered at an affected second group muscle, and wherein the total dose administered during the treatment is up to 300,000 pg, and
wherein the modified BoNT/A comprises a BoNT/A light-chain and translocation domain, and a BoNT/B receptor binding domain (H_{C} domain).

32. A method for treating paediatric limb spasticity, the method comprising administering a modified botulinum neurotoxin A (BoNT/A) by intramuscular injection to a plurality of affected muscles of a subject,
wherein the modified BoNT/A is administered by way of a unit dose of greater than 8,500 pg of modified BoNT/A at the plurality of affected muscles,
wherein the plurality of affected muscles are selected from:
a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius; and
wherein a single unit dose is administered at an affected first group muscle and/or multiple unit doses are administered at an affected second group muscle, and wherein the total dose administered during the treatment is up to 300,000 pg, and
wherein the modified BoNT/A comprises a BoNT/A light-chain and translocation domain, and a BoNT/B receptor binding domain (H_{C} domain).

33. Use of a modified botulinum neurotoxin A (BoNT/A) in the manufacture of a medicament for treating paediatric limb spasticity, wherein the modified BoNT/A is administered by intramuscular injection to a plurality of affected muscles of a subject,
wherein the modified BoNT/A is administered by way of a unit dose of greater than 8,500 pg of modified BoNT/A at the plurality of affected muscles,
wherein the plurality of affected muscles are selected from:
a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius; and
wherein a single unit dose is administered at an affected first group muscle and/or multiple unit doses are administered at an affected second group muscle, and wherein the total dose administered during the treatment is up to 300,000 pg, and
wherein the modified BoNT/A comprises a BoNT/A light-chain and translocation domain, and a BoNT/B receptor binding domain (H_{C} domain).

34. The modified BoNT/A for use, method or use according to any one of claims 31-33, wherein the unit dose is greater than 8,500 pg up to 20,000 pg (e.g. 8,501 pg up to 20,000 pg) of modified BoNT/A, preferably wherein the unit dose is greater than 8,500 pg up to 18,000 pg.

35. The modified BoNT/A for use, method or use according to any one of claims 31-34, wherein the unit dose is 10,000 pg to 15,000, preferably 12,000 pg to 13,000 pg, more preferably 12,500 pg, of modified BoNT/A.

36. The modified BoNT/A for use, method or use according to any one of claims 31-34, wherein the unit dose is 15,000 pg to 20,000, preferably 17,500 pg to 18,500 pg, more preferably 18,000 pg, of modified BoNT/A.

37. The modified BoNT/A for use, method or use according to any one of claims 31-34, wherein the unit dose is greater than 11,500 pg up to 19,000 pg, (e.g. 11,501 pg up to 19,000 pg) of modified BoNT/A.

38. The modified BoNT/A for use, method or use according to any one of claims 31-34 or 36-37, wherein the unit dose is greater than 17,000 pg up to 19,000 pg (e.g. 17,001 pg up to 19,000 pg) of modified BoNT/A.

39. The modified BoNT/A for use, method or use according to any one of claims 31-38, wherein the total dose administered is 125,000 pg to 300,000 pg (e.g. 127,500 pg to 300,000 pg) of modified BoNT/A.

40. The modified BoNT/A for use, method or use according to any one of claims 31-39, wherein the total dose administered is greater than 127,500 pg to 300,000 pg of modified BoNT/A.

41. The modified BoNT/A for use, method or use according to any one of claims 31-40, wherein the total dose administered is 175,000 to 300,000 pg of modified BoNT/A.

42. The modified BoNT/A for use, method or use according to any one of claims 31-40, wherein the total dose administered is up to 150,000 pg to 225,000 pg, preferably up to 180,000 pg to 195,000 pg, more preferably up to 187,500 pg, of modified BoNT/A.

43. The modified BoNT/A for use, method or use according to any one of claims 31-41, wherein the total dose administered is up to 225,000 pg to 300,000 pg, preferably up to 262,500 pg to 277,500 pg, more preferably up to 270,000 pg, of modified BoNT/A.

44. The modified BoNT/A for use, method or use according to any one of claims 31-41 or 43, wherein the total dose administered is 250,000 to 300,000 pg of modified BoNT/A.

45. A modified BoNT/A for use in treating paediatric limb spasticity, wherein the modified BoNT/A is administered by intramuscular injection to a plurality of affected muscles of a subject,
wherein the modified BoNT/A is administered by way of a unit dose greater than 353.5 Units of modified BoNT/A at the plurality of affected muscles, and wherein 1 Unit is an amount of the modified BoNT/A that corresponds to the calculated median lethal dose (LD₅₀) in mice,
wherein the plurality of affected muscles are selected from:
a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius; and
wherein a single unit dose is administered at an affected first group muscle and/or multiple unit doses are administered at an affected second group muscle, and wherein the total dose administered during the treatment is up to 12,479 Units, and
wherein the modified BoNT/A comprises a BoNT/A light-chain and translocation domain, and a BoNT/B receptor binding domain (H_{C} domain).

46. A method for treating paediatric limb spasticity, the method comprising administering a modified botulinum neurotoxin A (BoNT/A) by intramuscular injection to a plurality of affected muscles of a subject,
wherein the modified BoNT/A is administered by way of a unit dose greater than 353.5 Units of modified BoNT/A at the plurality of affected muscles, and wherein 1 Unit is an amount of the modified BoNT/A that corresponds to the calculated median lethal dose (LD₅₀) in mice,
wherein the plurality of affected muscles are selected from:
a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius; and
wherein a single unit dose is administered at an affected first group muscle and/or multiple unit doses are administered at an affected second group muscle, and wherein the total dose administered during the treatment is up to 12,479 Units, and
wherein the modified BoNT/A comprises a BoNT/A light-chain and translocation domain, and a BoNT/B receptor binding domain (H_{C} domain).

47. Use of a modified BoNT/A in the manufacture of a medicament for treating paediatric limb spasticity, wherein the modified BoNT/A is administered by intramuscular injection to a plurality of affected muscles of a subject,
wherein the modified BoNT/A is administered by way of a unit dose greater than 353.5 Units of modified BoNT/A at the plurality of affected muscles, and wherein 1 Unit is an amount of the modified BoNT/A that corresponds to the calculated median lethal dose (LD₅₀) in mice,
wherein the plurality of affected muscles are selected from:
a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius; and
wherein a single unit dose is administered at an affected first group muscle and/or multiple unit doses are administered at an affected second group muscle, and wherein the total dose administered during the treatment is up to 12,479 Units, and
wherein the modified BoNT/A comprises a BoNT/A light-chain and translocation domain, and a BoNT/B receptor binding domain (H_{C} domain).

48. The modified BoNT/A for use, method or use according to any one of claims 45-47, wherein the unit dose is greater than 353.5 Units up to 832 Units (e.g. 354 Units up to 832 Units) of modified BoNT/A, preferably wherein the unit dose is greater than 353.5 Units up to 749 Units (e.g. 354 Units up to 749 Units).

49. The modified BoNT/A for use, method or use according to any one of claims 45-48, wherein the unit dose is 416 Units to 624 Units, preferably 499 Units to 541 Units, more preferably 520 Units, of modified BoNT/A.

50. The modified BoNT/A for use, method or use according to any one of claims 45-48, wherein the unit dose is 624 Units to 832 Units, preferably 728 Units to 770 Units, more preferably 749 Units, of modified BoNT/A.

51. The modified BoNT/A for use, method or use according to any one of claims 45-48, wherein the unit dose is greater than 478 Units up to 790 Units, (e.g. 479 Units up to 790 Units) of modified BoNT/A.

52. The modified BoNT/A for use, method or use according to any one of claims 45-48, wherein the unit dose is greater than 707 Units up to 790 Units (e.g. 708 Units up to 790 Units) of modified BoNT/A.

53. The modified BoNT/A for use, method or use according to any one of claims 45-52, wherein the total dose administered is 5,200 Units to 12,479 Units (e.g. 5,304 Units to 12,479 Units) of modified BoNT/A.

54. The modified BoNT/A for use, method or use according to any one of claims 45-53, wherein the total dose administered is greater than 5,304 Units to 12,479 Units of modified BoNT/A.

55. The modified BoNT/A for use, method or use according to any one of claims 45-54, wherein the total dose administered is up to 6,240 Units to 9,360 Units, preferably up to 7,488 Units to 8,112 Units, more preferably up to 7,800 Units, of modified BoNT/A.

56. The modified BoNT/A for use, method or use according to any one of claims 45-54, wherein the total dose administered is 7,280 Units to 12,479 Units of modified BoNT/A.

57. The modified BoNT/A for use, method or use according to any one of claims 45-54 or 56, wherein the total dose administered is up to 9,360 Units to 12,479 Units, preferably up to 10,920 Units to 11,544 Units, more preferably up to 11,232 Units, of modified BoNT/A.

58. The modified BoNT/A for use, method or use according to any one of claims 45-54 or 56-57, wherein the total dose administered is 10,399 Units to 12,479 Units of modified BoNT/A.

59. The modified BoNT/A for use, method or use according to any one of claims 31-58,
wherein the paediatric limb spasticity is paediatric upper limb spasticity.

60. The modified BoNT/A for use, method, or use according to any one of the preceding claims, wherein:
the first group of affected muscles comprises: the flexor digitorum superficialis, the flexor digitorum profundus, the flexor carpi radialis, the flexor carpi ulnaris, the brachioradialis, the pronator teres, the biceps brachii, the gastrocnemius medial head, the gastrocnemius lateral head, the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
the second group of affected muscles comprises: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, and the gastrocnemius.

61. A modified botulinum neurotoxin A (BoNT/A) for use in treating limb spasticity (preferably adult limb spasticity), wherein the modified BoNT/A is administered by intramuscular injection to a plurality of affected muscles of a subject,
wherein the modified BoNT/A is administered by way of a unit dose of 10,000 pg to 20,000 pg (e.g. 14,000 pg to 16,000 pg, preferably 15,000 pg) of modified BoNT/A at the plurality of affected muscles,
wherein the plurality of affected muscles are selected from:
a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius, and
wherein a single unit dose is administered at an affected first group muscle and/or multiple unit doses are administered at an affected second group muscle, and wherein the total dose administered during the treatment is up to 300,000 pg (e.g. up to 240,000 pg, preferably up to 225,000 pg), and
wherein the modified BoNT/A comprises a BoNT/A light-chain and translocation domain, and a BoNT/B receptor binding domain (H_{C} domain).

62. A method for treating limb spasticity (preferably adult limb spasticity), the method comprising administering a modified botulinum neurotoxin A (BoNT/A) by intramuscular injection to a plurality of affected muscles of a subject,
wherein the modified BoNT/A is administered by way of a unit dose of 10,000 pg to 20,000 pg (e.g. 14,000 pg to 16,000 pg, preferably 15,000 pg) of modified BoNT/A at the plurality of affected muscles,
wherein the plurality of affected muscles are selected from:
a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius, and
wherein a single unit dose is administered at an affected first group muscle and/or multiple unit doses are administered at an affected second group muscle, and wherein the total dose administered during the treatment is up to 300,000 pg (e.g. up to 240,000 pg, preferably up to 225,000 pg), and
wherein the modified BoNT/A comprises a BoNT/A light-chain and translocation domain, and a BoNT/B receptor binding domain (H_{C} domain).

63. Use of a modified botulinum neurotoxin A (BoNT/A) in the manufacture of a medicament for treating limb spasticity (preferably adult limb spasticity), wherein the modified BoNT/A is administered by intramuscular injection to a plurality of affected muscles of a subject,
wherein the modified BoNT/A is administered by way of a unit dose of 10,000 pg to 20,000 pg (e.g. 14,000 pg to 16,000 pg, preferably 15,000 pg) of modified BoNT/A at the plurality of affected muscles,
wherein the plurality of affected muscles are selected from:
a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius, and
wherein a single unit dose is administered at an affected first group muscle and/or multiple unit doses are administered at an affected second group muscle, and wherein the total dose administered during the treatment is up to 300,000 pg (e.g. up to 240,000 pg, preferably up to 225,000 pg), and
wherein the modified BoNT/A comprises a BoNT/A light-chain and translocation domain, and a BoNT/B receptor binding domain (H_{C} domain).

64. A modified botulinum neurotoxin A (BoNT/A) for use in treating limb spasticity (preferably adult limb spasticity), wherein the modified BoNT/A is administered by intramuscular injection to a plurality of affected muscles of a subject,
wherein the modified BoNT/A is administered by way of a unit dose of 416 Units to 832 Units (e.g. 582 Units to 666 Units, preferably 624 Units) of modified BoNT/A at the plurality of affected muscles, and wherein 1 Unit is an amount of the modified BoNT/A that corresponds to the calculated median lethal dose (LD₅₀) in mice,
wherein the plurality of affected muscles are selected from:
a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius, and
wherein a single unit dose is administered at an affected first group muscle and/or multiple unit doses are administered at an affected second group muscle, and wherein the total dose administered during the treatment is up to 12,480 Units (e.g. up to 9,990 Units, preferably up to 9,360 Units), and
wherein the modified BoNT/A comprises a BoNT/A light-chain and translocation domain, and a BoNT/B receptor binding domain (H_{C} domain).

65. A method for treating limb spasticity (preferably adult limb spasticity), the method comprising administering a modified botulinum neurotoxin A (BoNT/A) by intramuscular injection to a plurality of affected muscles of a subject,
wherein the modified BoNT/A is administered by way of a unit dose of 416 Units to 832 Units (e.g. 582 Units to 666 Units, preferably 624 Units) of modified BoNT/A at the plurality of affected muscles, and wherein 1 Unit is an amount of the modified BoNT/A that corresponds to the calculated median lethal dose (LD₅₀) in mice,
wherein the plurality of affected muscles are selected from:
a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius, and
wherein a single unit dose is administered at an affected first group muscle and/or multiple unit doses are administered at an affected second group muscle, and wherein the total dose administered during the treatment is up to 12,480 Units (e.g. up to 9,990 Units, preferably up to 9,360 Units), and
wherein the modified BoNT/A comprises a BoNT/A light-chain and translocation domain, and a BoNT/B receptor binding domain (H_{C} domain).

66. Use of a modified botulinum neurotoxin A (BoNT/A) in the manufacture of a medicament for treating limb spasticity (preferably adult limb spasticity), wherein the modified BoNT/A is administered by intramuscular injection to a plurality of affected muscles of a subject,
wherein the modified BoNT/A is administered by way of a unit dose of 416 Units to 832 Units (e.g. 582 Units to 666 Units, preferably 624 Units) of modified BoNT/A at the plurality of affected muscles, and wherein 1 Unit is an amount of the modified BoNT/A that corresponds to the calculated median lethal dose (LD₅₀) in mice,
wherein the plurality of affected muscles are selected from:
a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius, and
wherein a single unit dose is administered at an affected first group muscle and/or multiple unit doses are administered at an affected second group muscle, and wherein the total dose administered during the treatment is up to 12,480 Units (e.g. up to 9,990 Units, preferably up to 9,360 Units), and
wherein the modified BoNT/A comprises a BoNT/A light-chain and translocation domain, and a BoNT/B receptor binding domain (H_{C} domain).

67. A modified botulinum neurotoxin A (BoNT/A) for use in treating paediatric limb spasticity, wherein the modified BoNT/A is administered by intramuscular injection to a plurality of affected muscles of a subject,
wherein the modified BoNT/A is administered by way of a unit dose of 5,000 pg to 10,000 pg (e.g. 7,000 pg to 8,000 pg, preferably 7,500 pg) of modified BoNT/A at the plurality of affected muscles,
wherein the plurality of affected muscles are selected from:
a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius, and
wherein a single unit dose is administered at an affected first group muscle and/or multiple unit doses are administered at an affected second group muscle, and wherein the total dose administered during the treatment is up to 150,000 pg (e.g. up to 120,000 pg, preferably up to 112,500 pg), and
wherein the modified BoNT/A comprises a BoNT/A light-chain and translocation domain, and a BoNT/B receptor binding domain (H_{C} domain).

68. A method for treating paediatric limb spasticity, the method comprising administering a modified botulinum neurotoxin A (BoNT/A) by intramuscular injection to a plurality of affected muscles of a subject,
wherein the modified BoNT/A is administered by way of a unit dose of 5,000 pg to 10,000 pg (e.g. 7,000 pg to 8,000 pg, preferably 7,500 pg) of modified BoNT/A at the plurality of affected muscles,
wherein the plurality of affected muscles are selected from:
a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius, and
wherein a single unit dose is administered at an affected first group muscle and/or multiple unit doses are administered at an affected second group muscle, and wherein the total dose administered during the treatment is up to 150,000 pg (e.g. up to 120,000 pg, preferably up to 112,500 pg), and
wherein the modified BoNT/A comprises a BoNT/A light-chain and translocation domain, and a BoNT/B receptor binding domain (H_{C} domain).

69. Use of a modified botulinum neurotoxin A (BoNT/A) in the manufacture of a medicament for treating paediatric limb spasticity, wherein the modified BoNT/A is administered by intramuscular injection to a plurality of affected muscles of a subject,
wherein the modified BoNT/A is administered by way of a unit dose of 5,000 pg to 10,000 pg (e.g. 7,000 pg to 8,000 pg, preferably 7,500 pg) of modified BoNT/A at the plurality of affected muscles,
wherein the plurality of affected muscles are selected from:
a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius, and
wherein a single unit dose is administered at an affected first group muscle and/or multiple unit doses are administered at an affected second group muscle, and wherein the total dose administered during the treatment is up to 150,000 pg (e.g. up to 120,000 pg, preferably up to 112,500 pg), and
wherein the modified BoNT/A comprises a BoNT/A light-chain and translocation domain, and a BoNT/B receptor binding domain (H_{C} domain).

70. A modified botulinum neurotoxin A (BoNT/A) for use in treating paediatric limb spasticity, wherein the modified BoNT/A is administered by intramuscular injection to a plurality of affected muscles of a subject,
wherein the modified BoNT/A is administered by way of a unit dose of 208 Units to 416 Units (e.g. 291 Units to 333 Units, preferably 312 Units) of modified BoNT/A at the plurality of affected muscles, and wherein 1 Unit is an amount of the modified BoNT/A that corresponds to the calculated median lethal dose (LD₅₀) in mice,
wherein the plurality of affected muscles are selected from:
a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius, and
wherein a single unit dose is administered at an affected first group muscle and/or multiple unit doses are administered at an affected second group muscle, and wherein the total dose administered during the treatment is up to 6,240 Units (e.g. up to 4,995 Units, preferably up to 4,680 Units), and
wherein the modified BoNT/A comprises a BoNT/A light-chain and translocation domain, and a BoNT/B receptor binding domain (H_{C} domain).

71. A method for treating paediatric limb spasticity, the method comprising administering a modified botulinum neurotoxin A (BoNT/A) by intramuscular injection to a plurality of affected muscles of a subject,
wherein the modified BoNT/A is administered by way of a unit dose of 208 Units to 416 Units (e.g. 291 Units to 333 Units, preferably 312 Units) of modified BoNT/A at the plurality of affected muscles, and wherein 1 Unit is an amount of the modified BoNT/A that corresponds to the calculated median lethal dose (LD₅₀) in mice,
wherein the plurality of affected muscles are selected from:
a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius, and
wherein a single unit dose is administered at an affected first group muscle and/or multiple unit doses are administered at an affected second group muscle, and wherein the total dose administered during the treatment is up to 6,240 Units (e.g. up to 4,995 Units, preferably up to 4,680 Units), and
wherein the modified BoNT/A comprises a BoNT/A light-chain and translocation domain, and a BoNT/B receptor binding domain (H_{C} domain).

72. Use of a modified botulinum neurotoxin A (BoNT/A) in the manufacture of a medicament for treating paediatric limb spasticity, wherein the modified BoNT/A is administered by intramuscular injection to a plurality of affected muscles of a subject,
wherein the modified BoNT/A is administered by way of a unit dose of 208 Units to 416 Units (e.g. 291 Units to 333 Units, preferably 312 Units) of modified BoNT/A at the plurality of affected muscles, and wherein 1 Unit is an amount of the modified BoNT/A that corresponds to the calculated median lethal dose (LD₅₀) in mice,
wherein the plurality of affected muscles are selected from:
a first group comprising: the flexor digitorum superficialis, the flexor digitorum profundus, the flexor carpi radialis, the flexor carpi ulnaris, the brachioradialis, the pronator teres, the biceps brachii, the gastrocnemius medial head (medial gastrocnemius), the gastrocnemius lateral head (lateral gastrocnemius), the flexor digitorum longus, the flexor hallucis longus, the gastrocnemius, the deltoid, the levator scapulae, the pronator quadratus, the flexor policis longus, the adductor policis, the flexor policis brevis, the palmaris longus, the lumbricales, the opponens policis, the adductor magnus, the adductor longus, the adductor brevis, the gracilis, the medial hamstrings, the lateral hamstrings, the tensor fascia lata, the rectus femoris, the vastus lateralis, the vastus medialis, the vastus intermedius, the gluteus maximus, the tibialis anterior, the flexor digitorum brevis, the extensor hallucis longus, and the flexor hallucis brevis; and
a second group comprising: the triceps brachii (long head), the subscapularis, the pectoralis (e.g. the pectoralis major), the latissimus dorsi, the biceps brachii, the brachialis, the soleus, the tibialis posterior, the brachioradialis, the teres major, the iliopsoas, the adductor magnus, a hamstring muscle (e.g. the biceps femoris, the semimembranosus, and/or the semitendinosus), the rectus femoris, and the gastrocnemius, and
wherein a single unit dose is administered at an affected first group muscle and/or multiple unit doses are administered at an affected second group muscle, and wherein the total dose administered during the treatment is up to 6,240 Units (e.g. up to 4,995 Units, preferably up to 4,680 Units), and
wherein the modified BoNT/A comprises a BoNT/A light-chain and translocation domain, and a BoNT/B receptor binding domain (H_{C} domain).

73. The modified BoNT/A for use, method, or use according to any one of the preceding claims, wherein the spasticity is upper limb spasticity and wherein the plurality of affected muscles are selected from:
a first group comprising (preferably consisting of): the flexor digitorum superficialis, the flexor digitorum profundus, the flexor carpi radialis, the flexor carpi ulnaris, and the brachioradialis; and
a second group comprising (preferably consisting of): the brachialis, the biceps brachii, the triceps brachii (long head), the subscapularis, the pectoralis (e.g. the pectoralis major), and the latissimus dorsi.

74. The modified BoNT/A for use, method, or use according to claim 73, wherein two unit doses are administered at the affected second group muscle.

75. The modified BoNT/A for use, method, or use according to any one of claims 1-29, 31-58, or 60-72, wherein at least one of the plurality of affected muscles is selected from: the adductor magnus, a hamstring muscle (e.g. the biceps femoris, the semimembranosus, and/or the semitendinosus), and the rectus femoris.

76. The modified BoNT/A for use, method, or use according to any one of claims 1-29, 31-58, 60-72, or 75, wherein the spasticity is lower limb spasticity and wherein the plurality of affected muscles are selected from:
a first group comprising (preferably consisting of): the gastrocnemius medial head (medial gastrocnemius), the gastrocnemius lateral head (lateral gastrocnemius), the tibialis anterior, the flexor digitorum longus, the flexor digitorum brevis, the flexor hallucis longus, flexor hallucis brevis, the gracilis, and the gluteus maximus; and
a second group comprising (preferably consisting of): the soleus, the tibialis posterior, the rectus femoris, a hamstring muscle (e.g. the biceps femoris, the semimembranosus, and/or the semitendinosus), and the adductor magnus.

77. The modified BoNT/A for use, method, or use according to claim 76, wherein when the second group muscle is:
(a) the soleus, three unit doses are administered thereto; or
(b) the tibialis posterior, the rectus femoris, a hamstring muscle (e.g. the biceps femoris, the semimembranosus, and/or the semitendinosus), or the adductor magnus, two unit doses are administered thereto.

78. The modified BoNT/A for use, method, or use according to any one of the preceding claims, wherein the total number of unit doses administered during the treatment is up to 15 unit doses.

79. The modified BoNT/A for use, method or use according to any one of the preceding claims, wherein the modified BoNT/A comprises a polypeptide sequence having at least 70% sequence identity to SEQ ID NO: 6.

80. The modified BoNT/A for use, method or use according to any one of the preceding claims, wherein the modified BoNT/A comprises the polypeptide sequence of SEQ ID NO: 6.

81. The modified BoNT/A for use, method or use according to any one of the preceding claims, wherein the modified BoNT/A consists of the polypeptide sequence of SEQ ID NO: 6.

82. The modified BoNT/A for use, method or use according to any one of the preceding claims, wherein the modified BoNT/A has a Safety Ratio of greater than 7, wherein the Safety Ratio is calculated as: dose of toxin required for −10% bodyweight change measured as pg/mouse divided by DAS ED₅₀ measured as pg/mouse, wherein ED₅₀=dose required to produce a DAS score of 2.

83. The modified BoNT/A for use, method, or use according to any one of the preceding claims, wherein the limb spasticity is treated for a longer duration than that treated by an unmodified BoNT/A (e.g. SEQ ID NO: 2 [such as a di-chain form of SEQ ID NO: 2]).

84. A unit dosage form of modified botulinum neurotoxin A (BoNT/A) (e.g. for treating adult limb spasticity), the unit dosage form comprising:
(a) greater than 17,000 pg of modified BoNT/A; or
(b) greater than 707 Units of modified BoNT/A, wherein 1 Unit is an amount of the modified BoNT/A that corresponds to the calculated median lethal dose (LD₅₀) in mice; and
(c) optionally a pharmaceutically acceptable carrier, excipient, adjuvant, and/or salt,
wherein the modified BoNT/A comprises a BoNT/A light-chain and translocation domain, and a BoNT/B receptor binding domain (H_{C} domain).

85. The unit dosage form according to claim 84, wherein the unit dosage form comprises 20,000 pg to 30,000 pg, preferably 24,000 pg to 26,000 pg, more preferably 25,000 pg, of modified BoNT/A.

86. The unit dosage form according to claim 84, wherein the unit dosage form comprises 832 Units to 1,248 Units, preferably 998 Units to 1,082 Units, more preferably 1,040 Units, of modified BoNT/A.

87. The unit dosage form according to claim 84, wherein the unit dosage form comprises 30,000 pg to 40,000 pg, preferably 35,000 pg to 37,000 pg, more preferably 36,000 pg, of modified BoNT/A.

88. The unit dosage form according to claim 84, wherein the unit dosage form comprises 1,248 Units to 1,664 Units, preferably 1,456 Units to 1,539 Units, more preferably 1,498 Units, of modified BoNT/A.

89. A unit dosage form of modified botulinum neurotoxin A (BoNT/A) for treating a paediatric subject (e.g. for treating paediatric limb spasticity), the unit dosage form comprising:
(a) greater than 8,500 pg of modified BoNT/A; or
(b) greater than 353.5 Units of modified BoNT/A, wherein 1 Unit is an amount of the modified BoNT/A that corresponds to the calculated median lethal dose (LD₅₀) in mice; and
(c) optionally a pharmaceutically acceptable carrier, excipient, adjuvant, and/or salt,
wherein the modified BoNT/A comprises a BoNT/A light-chain and translocation domain, and a BoNT/B receptor binding domain (H_{C} domain).

90. The unit dosage form according to claim 89, wherein the unit dosage form comprises 10,000 pg to 15,000 pg, preferably 12,000 pg to 13,000 pg, more preferably 12,500 pg, of modified BoNT/A

91. The unit dosage form according to claim 89, wherein the unit dosage form comprises 416 Units to 624 Units, preferably 499 Units to 541 Units, more preferably 520 Units, of modified BoNT/A.

92. The unit dosage form according to claim 89, wherein the unit dosage form comprises 15,000 pg to 20,000 pg, preferably 17,500 pg to 18,500 pg, more preferably 18,000 pg, of modified BoNT/A.

93. The unit dosage form according to claim 89, wherein the unit dosage form comprises 624 Units to 832 Units, preferably 728 Units to 770 Units, more preferably 749 Units, of modified BoNT/A.

94. The modified BoNT/A for use, method, use, or unit dosage form according to any one of the preceding claims, wherein the modified BoNT/A is a di-chain modified BoNT/A in which the L-chain is linked to the H-chain via a di-sulphide bond obtainable by a method comprising contacting a single-chain modified BoNT/A comprising (or consisting of) SEQ ID NO: 6 with a protease that hydrolyses a peptide bond in the activation loop thereof, thereby converting the single-chain modified BoNT/A into the corresponding di-chain modified BoNT/A.

95. The modified BoNT/A for use, method, use, or unit dosage form according to any one of the preceding claims, wherein an initial methionine amino acid residue of a polypeptide sequence of the modified BoNT/A is optional.

96. The modified BoNT/A for use, method, use, or unit dosage form according to any one of the preceding claims, wherein an initial methionine amino acid residue of a polypeptide sequence of the modified BoNT/A is absent.

97. The modified BoNT/A for use, method, use, or unit dosage form according to any one of the preceding claims, wherein the BoNT/B H_{C} domain comprises one or more substitution mutation(s) selected from the group consisting of: E1191M; S1199Y; V1118M; Y1183M; E11911; E1191Q; E1191T; S1199F; S1199L; S1201V; and combinations thereof, preferably wherein the BoNT/B H_{C} domain comprises substitution mutations E1191M and S1199Y.

98. The modified BoNT/A for use, method, use, or unit dosage form according to any one of the preceding claims, wherein the modified BoNT/A is a di-chain modified BoNT/A comprising (or consisting of) a light-chain comprising SEQ ID NO: 11 or 12 (preferably SEQ ID NO: 11) and a heavy-chain comprising SEQ ID NO: 13, wherein the light-chain and heavy-chain are joined together by a di-sulphide bond.

99. A kit comprising:
(a) the unit dosage form according to any one of claims 84-98; and
(b) instructions for use of the same in treating limb spasticity; and
(c) optionally a diluent.

100. A unit dosage form according to any one of claims 84-88 or 94-98 for use in treating adult limb spasticity.

101. A method for treating adult limb spasticity, the method comprising administering the dosage form according to any one of claims 84-88 or 94-98 to a subject.

102. Use of a unit dosage form according to any one of claims 84-88 or 94-98 in the manufacture of a medicament for treating adult limb spasticity.

103. A unit dosage form according to any one of claims 89-98 for use in treating paediatric limb spasticity.

104. A method for treating paediatric limb spasticity, the method comprising administering the dosage form according to any one of claims 89-98 to a subject.

105. Use of a unit dosage form according to any one of claims 89-98 in the manufacture of a medicament for treating paediatric limb spasticity.


SEQUENCE LISTING (13 sequences)

SEQ ID NO: 1 - DNA, 3888 bp, Clostridium botulinum
  source 1..3888: mol_type = other DNA
atgccattcgtcaacaagcaattcaactacaaagacccagtcaacggcgtcgacatcgcatacatcaagattccgaacgccggtcaaatgcagccggttaaggcttttaagatccacaacaagatttgggttatcccggagcgtgacaccttcacgaacccggaagaaggcgatctgaacccgccaccggaagcgaagcaagtccctgtcagctactacgattcgacgtacctgagcacggataacgaaaaagataactacctgaaaggtgtgaccaagctgttcgaacgtatctacagcacggatctgggtcgcatgctgctgactagcattgttcgcggtatcccgttctggggtggtagcacgattgacaccgaactgaaggttatcgacactaactgcattaacgttattcaaccggatggtagctatcgtagcgaagagctgaatctggtcatcattggcccgagcgcagacattatccaattcgagtgcaagagctttggtcacgaggttctgaatctgacccgcaatggctatggtagcacccagtacattcgtttttcgccggattttaccttcggctttgaagagagcctggaggttgataccaatccgttgctgggtgcgggcaaattcgctaccgatccggctgtcacgctggcccatgaactgatccacgcaggccaccgcctgtacggcattgccatcaacccaaaccgtgtgttcaaggttaatacgaatgcatactacgagatgagcggcctggaagtcagcttcgaagaactgcgcaccttcggtggccatgacgctaaattcattgacagcttgcaagagaatgagttccgtctgtactactataacaaattcaaagacattgcaagcacgttgaacaaggccaaaagcatcgttggtactaccgcgtcgttgcagtatatgaagaatgtgtttaaagagaagtacctgctgtccgaggatacctccggcaagtttagcgttgataagctgaagtttgacaaactgtacaagatgctgaccgagatttacaccgaggacaactttgtgaaattcttcaaagtgttgaatcgtaaaacctatctgaattttgacaaagcggttttcaagattaacatcgtgccgaaggtgaactacaccatctatgacggttttaacctgcgtaacaccaacctggcggcgaactttaacggtcagaatacggaaatcaacaacatgaatttcacgaagttgaagaacttcacgggtctgttcgagttctataagctgctgtgcgtgcgcggtatcatcaccagcaaaaccaaaagcctggacaaaggctacaacaaggcgctgaatgacctgtgcattaaggtaaacaattgggatctgttcttttcgccatccgaagataattttaccaacgacctgaacaagggtgaagaaatcaccagcgatacgaatattgaagcagcggaagagaatatcagcctggatctgatccagcagtactatctgacctttaacttcgacaatgaaccggagaacattagcattgagaatctgagcagcgacattatcggtcagctggaactgatgccgaatatcgaacgtttcccgaacggcaaaaagtacgagctggacaagtacactatgttccattacctgcgtgcacaggagtttgaacacggtaaaagccgtatcgcgctgaccaacagcgttaacgaggccctgctgaacccgagccgtgtctataccttcttcagcagcgactatgttaagaaagtgaacaaagccactgaggccgcgatgttcctgggctgggtggaacagctggtatatgacttcacggacgagacgagcgaagtgagcactaccgacaaaattgctgatattaccatcattatcccgtatattggtccggcactgaacattggcaacatgctgtacaaagacgattttgtgggtgccctgatcttctccggtgccgtgattctgctggagttcattccggagattgcgatcccggtgttgggtaccttcgcgctggtgtcctacatcgcgaataaggttctgacggttcagaccatcgataacgcgctgtcgaaacgtaatgaaaaatgggacgaggtttacaaatacattgttacgaattggctggcgaaagtcaatacccagatcgacctgatccgtaagaaaatgaaagaggcgctggagaatcaggcggaggccaccaaagcaattatcaactaccaatacaaccagtacacggaagaagagaagaataacattaacttcaatatcgatgatttgagcagcaagctgaatgaatctatcaacaaagcgatgatcaatatcaacaagtttttgaatcagtgtagcgtttcgtacctgatgaatagcatgattccgtatggcgtcaaacgtctggaggacttcgacgccagcctgaaagatgcgttgctgaaatacatttacgacaatcgtggtacgctgattggccaagttgaccgcttgaaagacaaagttaacaataccctgagcaccgacatcccatttcaactgagcaagtatgttgataatcaacgtctgttgagcactttcaccgagtatatcaaaaacatcatcaatactagcattctgaacctgcgttacgagagcaatcatctgattgatctgagccgttatgcaagcaagatcaacatcggtagcaaggtcaattttgacccgatcgataagaaccagatccagctgtttaatctggaatcgagcaaaattgaggttatcctgaaaaacgccattgtctacaactccatgtacgagaatttctccaccagcttctggattcgcatcccgaaatacttcaacagcattagcctgaacaacgagtatactatcatcaactgtatggagaacaacagcggttggaaggtgtctctgaactatggtgagatcatttggaccttgcaggacacccaagagatcaagcagcgcgtcgtgttcaagtactctcaaatgatcaacatttccgattacattaatcgttggatcttcgtgaccattacgaataaccgtctgaataacagcaagatttacatcaatggtcgcttgatcgatcagaaaccgattagcaacctgggtaatatccacgcaagcaacaacattatgttcaaattggacggttgccgcgatacccatcgttatatctggatcaagtatttcaacctgtttgataaagaactgaatgagaaggagatcaaagatttgtatgacaaccaatctaacagcggcattttgaaggacttctggggcgattatctgcaatacgataagccgtactatatgctgaacctgtatgatccgaacaaatatgtggatgtcaataatgtgggtattcgtggttacatgtatttgaagggtccgcgtggcagcgttatgacgaccaacatttacctgaactctagcctgtaccgtggtacgaaattcatcattaagaaatatgccagcggcaacaaagataacattgtgcgtaataacgatcgtgtctacatcaacgtggtcgtgaagaataaagagtaccgtctggcgaccaacgcttcgcaggcgggtgttgagaaaattctgagcgcgttggagatccctgatgtcggtaatctgagccaagtcgtggttatgaagagcaagaacgaccagggtatcactaacaagtgcaagatgaacctgcaagacaacaatggtaacgacatcggctttattggtttccaccagttcaacaatattgctaaactggtagcgagcaattggtacaatcgtcagattgagcgcagcagccgtactttgggctgtagctgggagtttatcccggtcgatgatggttggggcgaacgtccgctg

SEQ ID NO: 2 - protein, 1296 aa, Clostridium botulinum
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 3 - protein, 1315 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTEHHHHHHHHHH

SEQ ID NO: 4 - protein, 1327 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIIELGGGGSELSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTEHHHHHHHHHH

SEQ ID NO: 5 - protein, 1314 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTEHHHHHHHHHH

SEQ ID NO: 6 - protein, 1304 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 7 - protein, 1304 aa, synthetic construct
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 8 - protein, 1291 aa, Clostridium botulinum
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 9 - protein, 10 aa, synthetic construct
TKSLDKGYNK

SEQ ID NO: 10 - protein, 8 aa, synthetic construct
SLDKGYNK

SEQ ID NO: 11 - protein, 437 aa, synthetic construct
PFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSK

SEQ ID NO: 12 - protein, 439 aa, synthetic construct
PFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTK

SEQ ID NO: 13 - protein, 856 aa, synthetic construct
ALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTE